SEQUENCE LISTING (104 sequences)

SEQ ID NO: 1 - protein, 736 aa, Adeno-associated virus 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 2 - protein, 735 aa, Adeno-associated virus 2 [sequence omitted]

SEQ ID NO: 3 - protein, 736 aa, Adeno-associated virus 3A
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 4 - protein, 736 aa, Adeno-associated virus 3B
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 5 - protein, 734 aa, Adeno-associated virus 4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 6 - protein, 724 aa, Adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 7 - protein, 736 aa, Adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 8 - protein, 737 aa, Adeno-associated virus 7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 9 - protein, 738 aa, Adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 10 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - protein, 738 aa, Adeno-associated virus 10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGESESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 12 - protein, 7 aa, synthetic construct
  REGION 1..7: note = heterologous peptide insertion
HDITKNI

SEQ ID NO: 13 - protein, 7 aa, synthetic construct
  REGION 1..7: note = heterologous peptide insertion
NQDYTKT

SEQ ID NO: 14 - protein, 7 aa, synthetic construct
  REGION 1..7: note = heterologous peptide insertion
DNTVTRS

SEQ ID NO: 15 - protein, 7 aa, synthetic construct
  REGION 1..7: note = heterologous peptide insertion
SNSVQSI

SEQ ID NO: 16 - protein, 7 aa, synthetic construct
  REGION 1..7: note = heterologous peptide insertion
NSTRHTD

SEQ ID NO: 17 - protein, 7 aa, synthetic construct
  REGION 1..7: note = heterologous peptide insertion
TNRTSPD

SEQ ID NO: 18 - protein, 7 aa, synthetic construct
  REGION 1..7: note = heterologous peptide insertion
ISDQTKH

SEQ ID NO: 19 - protein, 7 aa, synthetic construct
  REGION 1..7: note = heterologous peptide insertion
QADTTKN

SEQ ID NO: 20 - protein, 7 aa, synthetic construct
  REGION 1..7: note = heterologous peptide insertion
NAVKTDF

SEQ ID NO: 21 - protein, 7 aa, synthetic construct
  REGION 1..7: note = heterologous peptide insertion
TNQTLSA

SEQ ID NO: 22 - protein, 7 aa, synthetic construct
  REGION 1..7: note = heterologous peptide insertion
ENRTTSN

SEQ ID NO: 23 - protein, 7 aa, synthetic construct
  REGION 1..7: note = heterologous peptide insertion
PQQDTTH

SEQ ID NO: 24 - protein, 7 aa, synthetic construct
  REGION 1..7: note = heterologous peptide insertion
NATNHVI

SEQ ID NO: 25 - protein, 7 aa, synthetic construct
  REGION 1..7: note = heterologous peptide insertion
TNNSKPD

SEQ ID NO: 26 - protein, 7 aa, synthetic construct
  REGION 1..7: note = heterologous peptide insertion
SKLTLNN

SEQ ID NO: 27 - protein, 7 aa, synthetic construct
  REGION 1..7: note = heterologous peptide insertion
VTAGMGA

SEQ ID NO: 28 - protein, 7 aa, synthetic construct
  REGION 1..7: note = heterologous peptide insertion
PNSTTNN

SEQ ID NO: 29 - protein, 7 aa, synthetic construct
  REGION 1..7: note = heterologous peptide insertion
NSTSRID

SEQ ID NO: 30 - protein, 7 aa, synthetic construct
  REGION 1..7: note = heterologous peptide insertion
VASHTNN

SEQ ID NO: 31 - protein, 7 aa, synthetic construct
  REGION 1..7: note = heterologous peptide insertion
RSHQEIP

SEQ ID NO: 32 - protein, 7 aa, synthetic construct
  REGION 1..7: note = heterologous peptide insertion
LNTTKDI

SEQ ID NO: 33 - protein, 7 aa, synthetic construct
  REGION 1..7: note = heterologous peptide insertion
IIDATKN

SEQ ID NO: 34 - protein, 7 aa, synthetic construct
  REGION 1..7: note = heterologous peptide insertion
NHISQTN

SEQ ID NO: 35 - protein, 7 aa, synthetic construct
  REGION 1..7: note = heterologous peptide insertion
SNSAHIT

SEQ ID NO: 36 - protein, 7 aa, synthetic construct
  REGION 1..7: note = heterologous peptide insertion
STHQSNN

SEQ ID NO: 37 - protein, 7 aa, synthetic construct
  REGION 1..7: note = heterologous peptide insertion
KTPNLTS

SEQ ID NO: 38 - protein, 7 aa, synthetic construct
  REGION 1..7: note = heterologous peptide insertion
SNTPALS

SEQ ID NO: 39 - protein, 7 aa, synthetic construct
  REGION 1..7: note = heterologous peptide insertion
SPGATTN

SEQ ID NO: 40 - protein, 10 aa, synthetic construct
  REGION 1..10: note = heterologous peptide insertion
LAHDITKNIA

SEQ ID NO: 41 - protein, 10 aa, synthetic construct
  REGION 1..10: note = heterologous peptide insertion
LANQDYTKTA

SEQ ID NO: 42 - protein, 10 aa, synthetic construct
  REGION 1..10: note = heterologous peptide insertion
LADNTVTRSA

SEQ ID NO: 43 - protein, 10 aa, synthetic construct
  REGION 1..10: note = heterologous peptide insertion
LASNSVQSIA

SEQ ID NO: 44 - protein, 10 aa, synthetic construct
  REGION 1..10: note = heterologous peptide insertion
LANSTRHTDA

SEQ ID NO: 45 - protein, 10 aa, synthetic construct
  REGION 1..10: note = heterologous peptide insertion
LATNRTSPDA

SEQ ID NO: 46 - protein, 10 aa, synthetic construct
  REGION 1..10: note = heterologous peptide insertion
LAISDQTKHA

SEQ ID NO: 47 - protein, 10 aa, synthetic construct
  REGION 1..10: note = heterologous peptide insertion
LAQADTTKNA

SEQ ID NO: 48 - protein, 10 aa, synthetic construct
  REGION 1..10: note = heterologous peptide insertion
LANAVKTDFA

SEQ ID NO: 49 - protein, 10 aa, synthetic construct
  REGION 1..10: note = heterologous peptide insertion
LATNQTLSAA

SEQ ID NO: 50 - protein, 10 aa, synthetic construct
  REGION 1..10: note = heterologous peptide insertion
LAENRTTSNA

SEQ ID NO: 51 - protein, 10 aa, synthetic construct
  REGION 1..10: note = heterologous peptide insertion
LAPQQDTTHA

SEQ ID NO: 52 - protein, 10 aa, synthetic construct
  REGION 1..10: note = heterologous peptide insertion
LANATNHVIA

SEQ ID NO: 53 - protein, 10 aa, synthetic construct
  REGION 1..10: note = heterologous peptide insertion
LATNNSKPDA

SEQ ID NO: 54 - protein, 10 aa, synthetic construct
  REGION 1..10: note = heterologous peptide insertion
LASKLTLNNA

SEQ ID NO: 55 - protein, 10 aa, synthetic construct
  REGION 1..10: note = heterologous peptide insertion
LAVTAGMGAA

SEQ ID NO: 56 - protein, 10 aa, synthetic construct
  REGION 1..10: note = heterologous peptide insertion
LAPNSTTNNA

SEQ ID NO: 57 - protein, 10 aa, synthetic construct
  REGION 1..10: note = heterologous peptide insertion
LANSTSRIDA

SEQ ID NO: 58 - protein, 10 aa, synthetic construct
  REGION 1..10: note = heterologous peptide insertion
LAVASHTNNA

SEQ ID NO: 59 - protein, 10 aa, synthetic construct
  REGION 1..10: note = heterologous peptide insertion
LARSHQEIPA

SEQ ID NO: 60 - protein, 10 aa, synthetic construct
  REGION 1..10: note = heterologous peptide insertion
LALNTTKDIA

SEQ ID NO: 61 - protein, 10 aa, synthetic construct
  REGION 1..10: note = heterologous peptide insertion
LAIIDATKNA

SEQ ID NO: 62 - protein, 10 aa, synthetic construct
  REGION 1..10: note = heterologous peptide insertion
LANHISQTNA

SEQ ID NO: 63 - protein, 10 aa, synthetic construct
  REGION 1..10: note = heterologous peptide insertion
LASNSAHITA

SEQ ID NO: 64 - protein, 10 aa, synthetic construct
  REGION 1..10: note = heterologous peptide insertion
LASTHQSNNA

SEQ ID NO: 65 - protein, 10 aa, synthetic construct
  REGION 1..10: note = heterologous peptide insertion
LAKTPNLTSA

SEQ ID NO: 66 - protein, 10 aa, synthetic construct
  REGION 1..10: note = heterologous peptide insertion
LASNTPALSA

SEQ ID NO: 67 - protein, 10 aa, synthetic construct
  REGION 1..10: note = heterologous peptide insertion
LASPGATTNA

SEQ ID NO: 68 - protein, 745 aa, synthetic construct
  REGION 1..745: note = Variant AAV Capsid
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLAHDITKNIARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 69 - protein, 745 aa, synthetic construct
  REGION 1..745: note = Variant AAV Capsid
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLANQDYTKTARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 70 - protein, 745 aa, synthetic construct
  REGION 1..745: note = Variant AAV Capsid
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLADNTVTRSARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 71 - protein, 745 aa, synthetic construct
  REGION 1..745: note = Variant AAV Capsid
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLASNSVQSIARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 72 - protein, 745 aa, synthetic construct
  REGION 1..745: note = Variant AAV Capsid
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLANSTRHTDARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 73 - protein, 745 aa, synthetic construct
  REGION 1..745: note = Variant AAV Capsid
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLATNRTSPDARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 74 - protein, 12 aa, synthetic construct
  REGION 1..12: note = heterologous peptide substitution
VPTGAETLNVNG

SEQ ID NO: 75 - protein, 10 aa, synthetic construct
  REGION 1..10: note = heterologous peptide substitution
LAPDFTTLDA

SEQ ID NO: 76 - protein, 12 aa, synthetic construct
  REGION 1..12: note = heterologous peptide substitution
SSTTAPATGTYN

SEQ ID NO: 77 - protein, 12 aa, synthetic construct
  REGION 1..12: note = heterologous peptide substitution
TGRQNPDMSGLS

SEQ ID NO: 78 - protein, 12 aa, synthetic construct
  REGION 1..12: note = heterologous peptide substitution
TGQRALDLRGLS

SEQ ID NO: 79 - protein, 12 aa, synthetic construct
  REGION 1..12: note = heterologous peptide substitution
TGWMSNQWLGLS

SEQ ID NO: 80 - protein, 12 aa, synthetic construct
  REGION 1..12: note = heterologous peptide substitution
TGVSQEPWAGLS

SEQ ID NO: 81 - protein, 12 aa, synthetic construct
  REGION 1..12: note = heterologous peptide substitution
TGVSLLVPSGLS

SEQ ID NO: 82 - protein, 12 aa, synthetic construct
  REGION 1..12: note = heterologous peptide substitution
TGGMGSWHSGLS

SEQ ID NO: 83 - protein, 12 aa, synthetic construct
  REGION 1..12: note = heterologous peptide substitution
TGSPLVFQAGLS

SEQ ID NO: 84 - protein, 12 aa, synthetic construct
  REGION 1..12: note = heterologous peptide substitution
TGLYDNSHVGLS

SEQ ID NO: 85 - protein, 12 aa, synthetic construct
  REGION 1..12: note = heterologous peptide substitution
TGDGDVGGGGLS

SEQ ID NO: 86 - protein, 12 aa, synthetic construct
  REGION 1..12: note = heterologous peptide substitution
TGPSPNPYTGLS

SEQ ID NO: 87 - protein, 12 aa, synthetic construct
  REGION 1..12: note = heterologous peptide substitution
TGNSGLAEAGLS

SEQ ID NO: 88 - protein, 12 aa, synthetic construct
  REGION 1..12: note = heterologous peptide substitution
TGLYSPNDGGLS

SEQ ID NO: 89 - protein, 13 aa, synthetic construct
  REGION 1..13: note = heterologous peptide substitution
PANPGTTATYLEG

SEQ ID NO: 90 - protein, 13 aa, synthetic construct
  REGION 1..13: note = heterologous peptide substitution
FSPTYPSVWWFQR

SEQ ID NO: 91 - protein, 13 aa, synthetic construct
  REGION 1..13: note = heterologous peptide substitution
VMPWGLVFVCFDF

SEQ ID NO: 92 - protein, 13 aa, synthetic construct
  REGION 1..13: note = heterologous peptide substitution
CMTAWPVDASFLN

SEQ ID NO: 93 - protein, 13 aa, synthetic construct
  REGION 1..13: note = heterologous peptide substitution
IYLRLGIYWCAGV

SEQ ID NO: 94 - protein, 13 aa, synthetic construct
  REGION 1..13: note = heterologous peptide substitution
GLGGSSTGSRTSA

SEQ ID NO: 95 - protein, 13 aa, synthetic construct
  REGION 1..13: note = heterologous peptide substitution
LFICFCCFYALFF

SEQ ID NO: 96 - protein, 13 aa, synthetic construct
  REGION 1..13: note = heterologous peptide substitution
IDDDCSVAGYRSW

SEQ ID NO: 97 - protein, 13 aa, synthetic construct
  REGION 1..13: note = heterologous peptide substitution
SNGITFKDRRCLL

SEQ ID NO: 98 - protein, 13 aa, synthetic construct
  REGION 1..13: note = heterologous peptide substitution
FMIGNKVPIALPG

SEQ ID NO: 99 - protein, 13 aa, synthetic construct
  REGION 1..13: note = heterologous peptide substitution
IYLRLGIYWCAGN

SEQ ID NO: 100 - protein, 12 aa, synthetic construct
  REGION 1..12: note = heterologous peptide substitution
PANPGTTATYLE

SEQ ID NO: 101 - protein, 12 aa, synthetic construct
  REGION 1..12: note = heterologous peptide insertion
LSTPFIVAGSGI

SEQ ID NO: 102 - DNA, 4443 bp, Homo sapiens
  source 1..4443: mol_type = other DNA
atgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatggtgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcttttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttag

SEQ ID NO: 103 - DNA, 3464 bp, Homo sapiens
  source 1..3464: mol_type = other DNA
atgcagcgcagcccactggagaaggcaagcgtggtgtccaagctgttcttttcctggaccaggcctatcctgaggaagggatacaggcagcggctggagctgagcgacatctatcagatcccttctgtggacagcgccgataatctgtccgagaagctggagagagagtgggatagggagctggcctctaagaagaacccaaagctgatcaatgccctgcggagatgcttcttttggcggttcatgttctacggcatcttcctgtatctgggcgaggtgaccaaggccgtgcagccactgctgctgggcagaatcatcgcctcttacgaccccgataacaaggaggagaggagcatcgccatctatctgggcatcggcctgtgcctgctgtttatcgtgaggacactgctgctgcacccagccatcttcggcctgcaccacatcggcatgcagatgagaatcgccatgttcagcctgatctacaagaagaccctgaagctgagctccagggtgctggacaagatctccatcggccagctggtgtccctgctgtctaacaatctgaacaagtttgatgagggactggccctggcacacttcgtgtggatcgcaccactgcaggtggccctgctgatgggcctgatctgggagctgctgcaggcaagcgccttttgcggactgggcttcctgatcgtgctggccctgttccaggcaggactgggacgcatgatgatgaagtacagagaccagagggccggcaagatctctgagcggctggtcatcaccagcgagatgatcgagaacatccagtccgtgaaggcctattgttgggaggaggccatggagaagatgatcgagaatctgcgccagacagagctgaagctgaccagaaaggccgcctacgtgaggtacttcaactctagcgccttctttttctctggctttttcgtggtgttcctgagcgtgctgccatacgccctgatcaagggcatcatcctgcggaagatctttaccacaatctccttctgcatcgtgctgagaatggccgtgacaaggcagtttccctgggccgtgcagacctggtatgactctctgggcgccatcaataagatccaggatttcctgcagaagcaggagtacaagacactggagtataacctgaccacaaccgaggtggtcatggagaatgtgaccgccttctgggaggagggctttggcgagctgttcgagaaggccaagcagaacaataacaatcgcaagacatctaacggcgacgatagcctgtttttcagcaatttttccctgctgggcacccccgtgctgaaggacatcaacttcaagatcgagaggggacagctgctggcagtggcaggctccacaggcgccggcaagacctctctgctgatgatgatcatgggcgagctggagccaagcgagggcaagatcaagcactccggccggatctctttttgcagccagttctcctggatcatgcccggcaccatcaaggagaatatcatctttggcgtgtcctacgatgagtacagatataggtctgtgatcaaggcctgtcagctggaggaggacatcagcaagttcgccgagaaggataacatcgtgctgggcgagggcggcatcacactgagcggaggacagagggcaaggatctccctggccagagccgtgtacaaggacgccgatctgtatctgctggacagcccctttggctatctggatgtgctgaccgagaaggagatcttcgagtcctgcgtgtgcaagctgatggccaataagacaaggatcctggtgacctctaagatggagcacctgaagaaggccgacaagatcctgatcctgcacgagggctcctcttacttttatggcacattcagcgagctgcagaatctgcagcctgacttcagctccaagctgatgggctgtgactcctttgatcagttctctgccgagaggcgcaactccatcctgacagagaccctgcacagattctctctggagggcgacgcacccgtgagctggacagagaccaagaagcagtcctttaagcagaccggcgagttcggcgagaagaggaagaattctatcctgaaccctatcaatagcacactgcaggcccggagaaggcagtctgtgctgaacctgatgacccacagcgtgaaccagggccagaatatccacagaaagacaaccgccagcacaaggaaggtgtccctggcacctcaggcaaacctgaccgagctggacatctactcccgccggctgtctcaggagaccggactggagatctctgaggagatcaatgaggaggatctgaaggagtgctttttcgacgatatggagagcatcccagccgtgacaacctggaacacatacctgcgctatatcaccgtgcacaagtccctgatctttgtgctgatctggtgtctggtcatcttcctggcagaggtggcagcatctctggtggtgctgtggctgctgggcaacacacccctgcaggacaagggcaattctacccacagccgcaacaattcctacgccgtgatcatcacatctacctctagctactacgtgttctacatctatgtgggcgtggccgatacactgctggccatgggctttttccggggcctgcccctggtgcacacactgatcaccgtgagcaagatcctgcaccacaagatgctgcacagcgtgctgcaggcccctatgtccacactgaacaccctgaaggccggcggcatcctgaatcggttttccaaggacatcgccatcctggacgatctgctgcctctgaccatctttgatttcatccagctgctgctgatcgtgatcggagcaatcgcagtggtggccgtgctgcagccttacatcttcgtggccacagtgccagtgatcgtggcctttatcatgctgcgcgcctatttcctgcagaccagccagcagctgaagcagctggagagcgagggccggtcccctatctttacacacctggtgacctccctgaagggactgtggacactgagggccttcggccggcagccatactttgagaccctgttccacaaggccctgaacctgcacacagccaattggtttctgtatctgagcaccctgcgctggtttcagatgcggatcgagatgatcttcgtgatctttttcatcgccgtgaccttcatctccatcctgacaaccggagagggagagggaagagtgggaatcatcctgacactggccatgaacatcatgtctaccctgcagtgggccgtgaattcctctatcgacgtggatagcctgatgagatctgtgagcagggtgtttaagttcatcgacatgcccacagagggcaagcctacaaagagcaccaagccatacaagaacggccagctgtccaaagtgatgatcatcgagaattctcacgtgaagaaggacgatatctggccatccgg

SEQ ID NO: 104 - DNA, 173 bp, synthetic construct
  misc_feature 1..173: note = CMV173 promoter
  source 1..173: mol_type = other DNA
actcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgt

(19) United States
(12) Patent Application Publication
(10) Pub. No.: US 2026/0014275 A1
(43) Pub. Date: Jan. 15, 2026

(54) Adeno-Associated Virus Variant Capsids with Improved Lung Tropism and Uses Thereof

(71) Applicant: 4D MOLECULAR THERAPEUTICS INC., Emeryville, CA (US)

(72) Inventors: Peter Francis, Emeryville, CA (US); Melissa A. KOTTERMAN, Emeryville, CA (US); Julie NYE, Emeryville, CA (US); Roxanne CROZE, Emeryville, CA (US)

(73) Assignee: 4D MOLECULAR THERAPEUTICS INC., Emeryville, CA (US)

(21) Appl. No.: 18/844,861

(22) PCT Filed: Mar. 6, 2023
(86) PCT No.: PCT/US2023/063789

§ 371 Date: Sep. 6, 2024

Related U.S. Application Data
(60) Provisional application No. 63/317,450, filed on Mar. 7, 2022.

(51) Int. Cl. A61K 48/00; A61K 38/17; A61K 38/57; C07K 14/005; C12N 15/86

(52) U.S. Cl. CPC A61K 48/005; A61K 38/1709; A61K 38/57; A61K 48/0075; C07K 14/005; C12N 15/86; C12N 2750/14122; C12N 2750/14143

(57) ABSTRACT

The present disclosure provides a variant AAV capsid protein that confers tropism to lung cells and recombinant adeno-associated viruses comprising the variant AAV capsid protein and pharmaceutical compositions comprising same and their use in the delivery of heterologous nucleic acids to lung cells for the treatment of pulmonary disorders.

### CROSS REFERENCE TO RELATED APPLICATIONS

This application claims the benefit of U.S. Provisional Patent Application Ser. No. 63/317,450, filed Mar. 7, 2022, the full disclosure of which is incorporated herein by reference.

### SEQUENCE LISTING SUBMISSION VIA EFS-WEB

A computer readable XML file, entitled “090400-5019-WO-Sequence-Listing” created on or about Mar. 3, 2023, with a file size of about 144,000 bytes contains the sequence listing for this application and is hereby incorporated by reference in its entirety.

### BACKGROUND OF THE INVENTION

Gene delivery vectors based on adeno-associated viruses (AAV) have demonstrated promise in both preclinical disease models and recently in human clinical trials for several disease targets. Vectors based on AAV are extremely safe because wild-type AAV is nonpathogenic and has no etiologic association with any known diseases. In addition. AAV offers the capability for highly efficient gene delivery and sustained transgene expression in numerous tissues, including liver, muscle, lung, retina, and brain.

AAV is a single stranded DNA virus that contains two open reading frames, rep and cap. The first gene encodes four proteins necessary for genome replication (Rep78, Rep68, Rep52, and Rep40), and the second expresses three structural proteins (VP1-3) that assemble to form the viral capsid. As its name implies, AAV is dependent upon the presence of a helper virus, such as an adenovirus or herpesvirus, for active replication. In the absence of a helper it establishes a latent state in which its genome is maintained episomally or integrated into the host chromosome. Multiple homologous primate AAV serotypes and numerous nonhuman primate serotypes have been identified. AAV2 is the best characterized as a gene delivery vehicle.

AAV has yielded promising results in an increasing number of clinical trials (Bainbridge et al., 2008; Carpentier et al., 2012; Gaudet et al., 2010; MacLaren et al., 2014; A. M. Maguire et al., 2009; A. Maguire & Simonelli, 2008; Nathwani et al., 2011, 2014; Stroes et al., 2008). However, there are impediments to gene delivery that may limit AAV's utility, such as anti-capsid immune responses, limited transduction of certain tissues, an inability for targeted delivery to specific cell types and a relatively low packaging capacity. In many situations, there is insufficient mechanistic knowledge to effectively empower rational design with the capacity to improve AAV. As an alternative, directed evolution has emerged as a strategy to create novel AAV variants that meet specific biomedical needs. Directed evolution strategies harness genetic diversification and selection processes to enable the accumulation of beneficial mutations that progressively improve the function of a biomolecule. In this process, wild-type AAV cap genes are diversified using several different approaches to create large genetic libraries that are packaged to generate libraries of viral particles, and selective pressure is then applied to isolate novel variants that can overcome gene delivery barriers.

Previously, proof-of-concept applications have shown that AAV directed evolution can be used to create optimized AAV variants for gene delivery to human lung epithelia in vitro or porcine lungs in vivo. These earlier studies demonstrate that an AAV variant optimized for lung epithelia transduction can lead to significantly improved gene therapy treatments for CF when evaluated in the context of the models in which they were selected. However, evaluation of these vectors has demonstrated species differences between pig and human lung epithelia that result in limited transduction of human lung epithelia by vectors evolved for pig lung epithelia transduction.

There is a need in the art for the development of novel AAV variants with improved delivery to human lung cells such as human lung epithelia for the treatment of pulmonary disorders.

### SUMMARY OF THE INVENTION

Provided herein are variant adeno-associated virus (AAV) capsid proteins having one or more modifications in amino acid sequence relative to a parental AAV capsid protein, which, when present in an AAV virion, confer increased infectivity of one or more types of lung cells as compared to the infectivity of the lung cells by an AAV virion comprising an unmodified parental AAV capsid protein. Also provided are recombinant AAV virions and pharmaceutical compositions thereof comprising a variant AAV capsid protein as described herein, methods of making variant rAAV capsid proteins and virions, and methods for using these rAAV capsid proteins and virions in research and in clinical practice, for example in the delivery of nucleic acid sequences to one or more cells of the lung for the treatment of lung disorders and diseases.

In some aspects of the disclosure, variant adeno-associated virus (AAV) capsid proteins are provided, these variant AAV capsid proteins having one or more modifications in amino acid sequence relative to a parental AAV capsid, which, when present in an AAV virion, confer increased infectivity of one or more types of lung cells (e.g., an airway epithelial cell, including but not limited to an alveolar epithelium cell, a bronchial (primary, secondary or tertiary) epithelial cell or a tracheal epithelial cell, a ciliated airway epithelial cell, a lung alveolar epithelial type 1 (AECI) or type 2 (AECII) cell, a smooth muscle or an endothelial cell) as compared to the infectivity of the lung cells by an AAV virion comprising a parental AAV capsid protein that does not comprise the amino acid sequence modification.

In some aspects of the disclosure, recombinant AAV (rAAV) virions are provided, these rAAV virions comprising a variant capsid protein as described herein, wherein the rAAV virions exhibit increased infectivity of one or more types of lung cells relative to the infectivity of the lung cell by an AAV virion comprising a corresponding unmodified parental AAV capsid protein. In some embodiments, the rAAV virion exhibits increased infectivity of all lung cells relative to the AAV virion comprising the parental AAV capsid protein. In other embodiments, the rAAV virion exhibits increased infectivity of certain cell types of the lung but not others relative of the AAV virion comprising the parental AAV capsid protein. Put another way, the rAAV virion exhibits increased infectivity that is preferential for certain cell types of the lung but not others. e.g., the rAAV demonstrates a preferentially increased infectivity of one or more lung upper airway cells, but does not demonstrate increased infectivity of all cell types. In some aspects, a variant capsid protein as herein described confers to these rAAV virions an increased resistance to human AAV neutralizing antibodies.

In some embodiments, the rAAV virion comprises a heterologous nucleic acid. In some such embodiments, the heterologous nucleic acid encodes an RNA that encodes a polypeptide. In other such embodiments, the heterologous nucleic acid sequence encodes an RNA that does not encode a polypeptide, e.g., the heterologous nucleic acid sequence an RNA interference agent, a guide RNA for a nuclease, etc. In some embodiments, the heterologous nucleic acid comprises a nucleotide encoding a polypeptide and a nucleotide sequence encoding an interfering RNA.

Also provided herein are pharmaceutical compositions comprising the subject infectious rAAV virions and a pharmaceutically acceptable carrier. In some embodiments, the pharmaceutical composition is formulated as a liquid/suspension suitable for aerosolized delivery. In related embodiments, the pharmaceutical composition is administered as an aerosol suspension of respirable particles comprising the rAAV virions, which the subject inhales. The respirable particles may be liquid or solid. Aerosols of liquid particles comprising the rAAV virions may be produced by any suitable means such as with a pressure-driven aerosol nebulizer or an ultrasonic nebulizer. Aerosols of solid particles comprising the rAAV virions may be produced with any solid particulate aerosol generator.

Also provided is the use of an rAAV virion comprising a variant capsid protein as herein described in a method of delivering a heterologous nucleic acid to a target cell (such as a lung cell) by contacting the target cell with the rAAV virion. In some embodiments, the target cell is in vivo, such as in the lung of an individual in need of treatment for a lung disease. In other embodiments, the target cell is in vitro.

Also provided are methods of treating a lung disease by administering to a subject in need of such treatment an effective amount of rAAV virions comprising a variant capsid protein as herein described or a pharmaceutical composition comprising an effective amount of the rAAV virions.

Also provided is an isolated nucleic acid comprising a sequence encoding a variant AAV capsid protein as described herein and a host cell comprising the isolated nucleic acid. In yet other embodiments, the isolated nucleic acid and/or isolated host cell comprises the rAAV.

In some aspects, the variant AAV capsid protein comprises an insertion of from about 5 amino acids to about 20 amino acids (a “heterologous peptide”, or “peptide insertion”) in the GH-loop of the capsid protein, preferably in a surface-exposed region of the GH-loop, relative to a corresponding parental AAV capsid protein, wherein the variant capsid protein, when present in an AAV virion, confers increased infectivity of a retinal cell compared to the infectivity of a retinal cell by an AAV virion comprising the corresponding parental AAV capsid protein. In some embodiments, the peptide comprises, consists essentially of, or consists of a sequence selected from the group consisting of:

(SEQ ID NO: 12)
HDITKNI,
(SEQ ID NO: 13)
NQDYTKT,
(SEQ ID NO: 14)
DNTVTRS,
(SEQ ID NO: 15)
SNSVQSI,
(SEQ ID NO: 16)
NSTRHTD,
(SEQ ID NO: 17)
TNRTSPD,
(SEQ ID NO: 18)
ISDQTKH,
(SEQ ID NO: 19)
QADTTKN,
(SEQ ID NO: 20)
NAVKTDF,
(SEQ ID NO: 21)
TNQTLSA,
(SEQ ID NO: 22)
ENRTTSN,
(SEQ ID NO: 23)
PQQDTTH,
(SEQ ID NO: 24)
NATNHVI,
(SEQ ID NO: 25)
TNNSKPD,
(SEQ ID NO: 26)
SKLTLNN,
(SEQ ID NO: 27)
VTAGMGA,
(SEQ ID NO: 28)
PNSTINN,
(SEQ ID NO: 29)
NSTSRID,
(SEQ ID NO: 30)
VASHINN,
(SEQ ID NO: 31)
RSHQEIP,
(SEQ ID NO: 32)
LNTTKDI,
(SEQ ID NO: 33)
IIDATKN,
(SEQ ID NO: 34)
NHISQTN,
(SEQ ID NO: 35)
SNSAHIT,
(SEQ ID NO: 36)
STHQSNN,
(SEQ ID NO: 37)
KTPNLTS,
(SEQ ID NO: 38)
SNTPALS,
(SEQ ID NO: 39)
SPGATTN,
(SEQ ID NO: 40)
LAHDITKNIA,
(SEQ ID NO: 41)
LANQDYTKTA,
(SEQ ID NO: 42)
LADNTVTRSA,
(SEQ ID NO: 43)
LASNSVQSIA,
(SEQ ID NO: 44)
LANSTRHTDA,
(SEQ ID NO: 45)
LATNRTSPDA,
(SEQ ID NO: 46)
LAISDQTKHA,
(SEQ ID NO: 47)
LAQADTTKNA,
(SEQ ID NO: 48)
LANAVKTDFA,
(SEQ ID NO: 49)
LATNQTLSAA,
(SEQ ID NO: 50)
LAENRTTSNA,
(SEQ ID NO: 51)
LAPQQDTTHA,
(SEQ ID NO: 52)
LANATNHVIA,
(SEQ ID NO: 53)
LATNNSKPDA,
(SEQ ID NO: 54)
LASKLTLNNA,
(SEQ ID NO: 55)
LAVTAGMGAA,
(SEQ ID NO: 56)
LAPNSTTNNA,
(SEQ ID NO: 57)
LANSTSRIDA,
(SEQ ID NO: 58)
LAVASHINNA,
(SEQ ID NO: 59)
LARSHQEIPA,
(SEQ ID NO: 60)
LALNTTKDIA,
(SEQ ID NO: 61)
LAIIDATKNA,
(SEQ ID NO: 62)
LANHISQTNA,
(SEQ ID NO: 63)
LASNSAHITA,
(SEQ ID NO: 64)
LASTHQSNNA,
(SEQ ID NO: 65)
LAKTPNLTSA,
(SEQ ID NO: 66)
LASNTPALSA
and
(SEQ ID NO: 67)
LASPGATTNA.

In some aspects, the variant AAV capsid protein comprises a peptide insertion in the GH-loop of the capsid protein relative to a corresponding parental AAV capsid protein and further comprises one or more amino acid substitutions relative to a corresponding parental AAV capsid protein, wherein the variant capsid protein, when present in an AAV virion, confers increased infectivity of a lung cell compared to the infectivity of a lung cell by an AAV virion comprising the corresponding parental AAV capsid protein. In some preferred aspects, the variant AAV capsid protein comprises, consists essentially of, or consists of a sequence selected from the group consisting of: HDITKNI (SEQ ID NO:12). NQDYTKT (SEQ ID NO:13), DNTVTRS (SEQ ID NO:14), SNSVQSI (SEQ ID NO:15), NSTRHTD (SEQ ID NO:16), TNRTSPD (SEQ ID NO:17), ISDQTKH (SEQ ID NO:18), QADTTKN (SEQ ID NO: 19), NAVKTDF (SEQ ID NO:20), TNQTLSA (SEQ ID NO:21), ENRTTSN (SEQ ID NO:22), PQQDTTH (SEQ ID NO:23), NATNHVI (SEQ ID NO:24), TNNSKPD (SEQ ID NO:25), SKLTLNN (SEQ ID NO:26), VTAGMGA (SEQ ID NO: 27), PNSTTNN (SEQ ID NO:28), NSTSRID (SEQ ID NO:29), VASHTNN (SEQ ID NO: 30), RSHQEIP (SEQ ID NO:31), LNTTKDI (SEQ ID NO:32), IIDATKN (SEQ ID NO: 33), NHISQTN (SEQ ID NO:34), SNSAHIT (SEQ ID NO:35), STHQSNN (SEQ ID NO: 36), KTPNLTS (SEQ ID NO:37), SNTPALS (SEQ ID NO:38), SPGATTN (SEQ ID NO: 39), LAHDITKNIA (SEQ ID NO:40), LANQDYTKTA (SEQ ID NO:41), LADNTVTRSA (SEQ ID NO:42), LASNSVQSIA (SEQ ID NO:43), LANSTRHTDA (SEQ ID NO:44), LATNRTSPDA (SEQ ID NO:45), LAISDQTKHA (SEQ ID NO:46), LAQADTTKNA (SEQ ID NO:47), LANAVKTDFA (SEQ ID NO:48), LATNQTLSAA (SEQ ID NO:49), LAENRTTSNA (SEQ ID NO:50), LAPQQDTTHA (SEQ ID NO:51), LANATNHVIA (SEQ ID NO:52), LATNNSKPDA (SEQ ID NO:53), LASKLTLNNA (SEQ ID NO:54), LAVTAGMGAA (SEQ ID NO:55), LAPNSTTNNA (SEQ ID NO:56), LANSTSRIDA (SEQ ID NO:57), LAVASHTNNA (SEQ ID NO:58), LARSHQEIPA (SEQ ID NO:59), LALNTTKDIA (SEQ ID NO:60), LAIIDATKNA (SEQ ID NO:61), LANHISQTNA (SEQ ID NO:62), LASNSAHITA (SEQ ID NO:63), LASTHQSNNA (SEQ ID NO:64), LAKTPNLTSA (SEQ ID NO:65), LASNTPALSA (SEQ ID NO:66) and LASPGATTNA (SEQ ID NO:67) and further comprises a V708I substitution relative to VP1 of AAV2.

Also disclosed herein are methods for manufacture and/or delivery of an rAAV comprising a variant AAV capsid as disclosed herein. In addition, provided herein are kits comprising an rAAV comprising a variant AAV capsid as disclosed herein and for use in methods described herein.

In other embodiments, the AAV virion comprising the variant capsid protein in the preceding paragraphs may incorporate any of the preceding or subsequently disclosed embodiments. Indeed, it is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the invention and are disclosed herein just as if each and every combination was individually and explicitly disclosed. In addition, all subcombinations of the various embodiments and elements thereof are also specifically embraced by the invention and are disclosed herein just as if each and every such subcombination was individually and explicitly disclosed herein.

### DESCRIPTION OF THE DRAWINGS

FIG. 1 illustrates the directed evolution process used to identify variant AAV capsids conferring improved airway transduction.

FIG. 2 is a schematic representation of a model system decision tree. Decision was based on sequencing analysis performed after the second and third rounds of NHP delivery.

FIGS. 3A-B FIG. 3a illustrates estimated genetic diversity of the capsid libraries, with a total diversity of >1 billion variants. FIG. 3b illustrates productivity of the capsid libraries, all of which were manufactured at a level sufficient to produce material for the in vivo therapeutic vector evolution process. The viral genomes (vg) administered represent the target dose, not accounting for losses associated with the delivery device and route of administration.

FIG. 4 illustrates ddPCR quantification of viral genomes present in the trachea and primary, secondary, and tertiary bronchi of NHPs administered the capsid libraries for rounds 1-3 of the selection process.

FIG. 5 illustrates the frequency of “hits” within sequencing analysis of round 3. Sequencing analysis was based on total frequency within sequenced population for each region. The left panel (trachea and primary bronchi) frequencies are as follows: (i) Point Mutant #1=13%; (ii) Peptide Insertion #1=11%; (iii) Peptide Insertion #2=4%; (iv) Peptide Insertion #3=6%; (v) AAV2 Point Mutants=11%; (vi) AAV2 Peptide Insertions=40%; (vii) Chimera=7%; (viii) Other=8%. The right panel (secondary and tertiary bronchi) frequencies are as follows: (i) Point Mutant #1=9%; (ii) Peptide Insertion #1=3%; (iii) Peptide Insertion #2=10%; (iv) Peptide Insertion #3=3%; (v) Peptide Insertion #4-7%; (vi) AAV2 Point Mutants=9%; (vii) AAV2 Peptide Insertions=54%; (viii) Other=5%.

FIGS. 6A-B illustrates ddPCR quantification of viral genomes present in human upper airway ALI cultures from the administered capsid libraries, in the absence and presence of human IVIG for Rounds 4 (FIG. 6a) and 5 (FIG. 6b) of the selection.

FIG. 7 illustrates the frequency of “hits” within sequencing analysis of round 5. Sequencing analysis was based on total frequency within sequenced population for selections in the presence and absence of IVIG. The left panel (in vitro Round 5A −IVIG) frequencies are as follows: (i) Point Mutant #1=10%; (ii) Peptide Insertion #1=17%; (iii) Peptide Insertion #2=8%; (iv) Peptide Insertion #3=1%; (v) Peptide Insertion #4=4%; (vi) Peptide Insertion #5=9%; (vi) Peptide Insertion #6=15%; (vii) AAV2 Peptide Insertions=35%; (viii) Other=1%. The right panel (in vitro Round 5B +IVIG) frequencies are as follows: (i) Point Mutant #1=6.7%; (ii) Peptide Insertion #1=11.5%; (iii) Peptide Insertion #2=9.6%; (iv) Peptide Insertion #3=2.9%; (v) Peptide Insertion #4=1.9%; (vi) Peptide Insertion #5=16.3%; (vi) Peptide Insertion #6=10.6%; (vii) AAV2 Peptide Insertions=40.4%.

FIGS. 8A-C AAV capsid transduction efficiency in lung upper airway epithelial ALI cultures measured by reporter EGFP expression using fluorescent microscopy. FIGS. 8a-b show human (FIG. 8a) and NHP (FIG. 8b) apical transduction of six novel variants compared to AAV2, AAV5 and AAV101. FIG. 8c shows human basal transduction of six novel variants compared to AAV2, AAV5 and AAV101. Adeno-associated virus (AAV), air-liquid interface (ALI), non-transduced (NT), multiplicity of infection (MOI).

FIGS. 9A-C Secondary analysis of top novel capsids compared to AAV2 and AAV101 by immunocytochemistry (FIGS. 9a, 9b) and ddPCR (FIG. 9c). AAV capsid transduction efficiency in lung upper airway epithelial ALI cultures measured by reporter EGFP expression using fluorescent microscopy (FIG. 9a). Quantification of micrograph pixel intensity (FIG. 9b). Transcript quantification following transduction (FIG. 9c). Adeno-associated virus (AAV), air-liquid interface (ALI), non-transduced (NT), multiplicity of infection (MOI), mean fluorescent intensity (MFI), housekeeper (HK). Error bars=standard deviation. n=3 per condition.

FIGS. 10A-B illustrate EGFP expression in human airway epithelial cell cultures following transduction with the specified AAV capsids carrying a nucleic acid comprising an EGFP reporter gene operably linked to a CAG promoter, in the presence or absence of mucus. Cultures were grown for ≥30 days in air liquid interface; post infection time 7 days;-mucus, without mucus: +mucus, with mucus: apical transduction; multiplicity of infection (MOI) 25,000

### DETAILED DESCRIPTION OF THE INVENTION

### Definitions

As used in the specification and claims, the singular form “a”, “an”, and “the” include plural references unless the context clearly dictates otherwise. For example, the term “a cell” includes a plurality of cells, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. For example, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions disclosed herein. Aspects defined by each of these transition terms are within the scope of the present disclosure.

Adeno-associated virus is a nonpathogenic parvovirus composed of a 4.7 kb single-stranded DNA genome within a non-enveloped, icosahedral capsid. The genome contains three open reading frames (ORF) flanked by inverted terminal repeats (ITR) that function as the viral origin of replication and packaging signal. The rep ORF encodes four nonstructural proteins that play roles in viral replication, transcriptional regulation, site-specific integration, and virion assembly. The cap ORF encodes three structural proteins (VP 1-3) that assemble to form a 60-mer viral capsid. Finally, an ORF present as an alternate reading frame within the cap gene produces the assembly-activating protein (AAP), a viral protein that localizes AAV capsid proteins to the nucleolus and functions in the capsid assembly process.

There are several naturally occurring (“wild-type”) serotypes and over 100 known variants of AAV, each of which differs in amino acid sequence, particularly within the hypervariable regions of the capsid proteins, and thus in their gene delivery properties. No AAV has been associated with any human disease, making recombinant AAV attractive for clinical applications.

For the purposes of the disclosure herein, the terminology “AAV” is an abbreviation for adeno-associated virus, including, without limitation, the virus itself and derivatives thereof, Except where otherwise indicated, the terminology refers to all subtypes or serotypes and both replication-competent and recombinant forms. The term “AAV” includes, without limitation, AAV type 1 (AAV-1 or AAV1), AAV type 2 (AAV-2 or AAV2). AAV type 3A (AAV-3A or AAV3A), AAV type 3B (AAV-3B or AAV3B), AAV type 4 (AAV-4 or AAV4), AAV type 5 (AAV-5 or AAV5), AAV type 6 (AAV-6 or AAV6), AAV type 7 (AAV-7 or AAV7), AAV type 8 (AAV-8 or AAV8), AAV type 9 (AAV-9 or AAV9), AAV type 10 (AAV-10 or AAV10 or AAVrh10), avian AAV, bovine AAV, canine AAV, caprine AAV, equine AAV, primate AAV, non-primate AAV, and ovine AAV. “Primate AAV” refers to AAV that infect primates, “non-primate AAV” refers to AAV that infect nonprimate mammals, “bovine AAV” refers to AAV that infect bovine mammals, etc.

The genomic sequences of various serotypes of AAV, as well as the sequences of the native terminal repeats (TRs), Rep proteins, and capsid subunits are known in the art. Such sequences may be found in the literature or in public databases such as GenBank. See, e.g., GenBank Accession Numbers NC_002077.1 (AAV1). AF063497.1 (AAV1), NC_001401.2 (AAV2), AF043303.1 (AAV2), J01901.1 (AAV2), U48704.1 (AAV3A), NC_001729.1 (AAV3A), AF028705.1 (AAV3B), NC_001829.1 (AAV4), U89790.1 (AAV4). NC_006152.1 (AAV5), AF085716.1 (AAV-5), AF028704.1 (AAV6), NC_006260.1 (AAV7), AF513851.1 (AAV7), AF513852.1 (AAV8) NC_006261.1 (AAV-8), AY530579.1 (AAV9), AAT46337 (AAV10) and AA088208 (AAVrhlO); the disclosures of which are incorporated by reference herein for teaching AAV nucleic acid and amino acid sequences. See also, e.g., Srivistava et al. (1983) J. Virology 45:555; Chiorini et al. (1998) J, Virology 71:6823; Chiorini et al. (1999) J. Virology 73:1309; Bantel-Schaal et al. (1999) J. Virology 73:939; Xiao et al. (1999) J. Virology 73:3994; Muramatsu et al. (1996) Virology 221:208; Shade et. al. (1986) J. Virol. 58:921; Gao et al. (2002) Proc. Nat. Acad. Sci. USA 99:11854; Moris et al. (2004) Virology 33:375-383; international patent publications WO 00/28061. WO 99/61601. WO 98/11244; and U.S. Pat. No. 6,156,303.

The sequences of naturally existing cap (capsid) proteins associated with AAV serotypes are known in the art and include those disclosed herein as AAV1 (SEQ ID NO: 1), AAV2 (SEQ ID NO:2), AAV3A (SEQ ID NO:3), AAV3B (SEQ ID NO:4), AAV4 (SEQ ID NO:5), AAV5 (SEQ ID NO:6), AAV6 (SEQ ID NO:7), AAV7 (SEQ ID NO:8), AAV8 (SEQ ID NO:9), AAV9 (SEQ ID NO: 10), and AAV10 (SEQ ID NO:11). The terms “variant AAV capsid protein” or “AAV variant” refer to an AAV capsid protein comprising an amino acid sequence that includes at least one modification or substitution (including deletion, insertion, point mutation, etc.) relative to a naturally existing or “wild-type” AAV capsid protein sequences, e.g., as set forth in SEQ ID NO: 1-11 herein. A variant AAV capsid protein may have about 80% identity or more to the amino acid sequence of a wild type capsid protein, for example, 85% identity or more, 90% identity or more, or 95% identity or more to the amino acid sequence of the wild type capsid protein, for example, 98% or 99% identity to the wild type capsid protein. A variant AAV capsid protein may not be a wild type capsid protein.

For the purposes of the disclosure herein, “AAV virion” or “AAV viral particle” refers to a viral particle composed of at least one AAV capsid protein and an encapsidated AAV polynucleotide.

For the purposes of the disclosure herein, the terminology “TAAV” is an abbreviation that refers to recombinant adeno-associated virus. “Recombinant,” as applied to a polynucleotide means that the polynucleotide is the product of various combinations of cloning, restriction or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

The term “rAAV vector” encompasses rAAV virions rAAV viral particles) (e.g., an infectious rAAV virion), which by definition include an rAAV polynucleotide; and also encompasses polynucleotides encoding rAAV (e.g, a single stranded polynucleotide encoding rAAV (ss-rAAV): a double stranded polynucleotide encoding rAAV (ds-rAAV), e.g., plasmids encoding rAAV; and the like).

If an AAV virion comprises a heterologous polynucleotide (i.e., a polynucleotide other than a wild-type AAV genome, e.g., a transgene to be delivered to a target cell, an RNAi agent or CRISPR agent to be delivered to a target cell, etc.), it is typically referred to as a “recombinant AAV (rAAV) virion” or an “TAAV viral particle.” In general, the heterologous polynucleotide is flanked by at least one, and generally by two. AAV inverted terminal repeat sequences (I′I′Rs).

The term “packaging” refers to a series of intracellular events that result in the assembly and encapsidation of an AAV particle. AAV “rep” and “cap” genes refer to polynucleotide sequences encoding replication and encapsidation proteins of adeno-associated virus. AAV rep and cap are referred to herein as AAV “packaging genes.”

The terminology “helper virus” for AAV refers to a virus that allows AAV (e.g., wild-type AAV) to be replicated and packaged by a mammalian cell. A variety of such helper viruses for AAV are known in the art, including adenoviruses, herpesviruses and poxviruses such as vaccinia. The adenoviruses encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

The terminology “helper virus function(s)” refers to function(s) encoded in a helper virus genome which allow AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans. For example, a plasmid or other expression vector comprising nucleotide sequences encoding one or more adenoviral proteins is transfected into a producer cell along with an rAAV vector.

The terminology “infectious” virus or viral particle is one that comprises a competently assembled viral capsid and is capable of delivering a polynucleotide component into a cell for which the viral species is tropic. The term does not necessarily imply any replication capacity of the virus. Assays for counting infectious viral particles are described elsewhere in this disclosure and in the art. Viral infectivity can be expressed as the ratio of infectious viral particles to total viral particles. Methods of determining the ratio of infectious viral particle to total viral particle are known in the art, See, e.g., Grainger et al. (2005) Mol, Ther. 11: S337 (describing a TCID50 infectious titer assay); and Zolotukhin et al. (1999) Gene Ther. 6:973. See also the Examples.

The term “tropism” as used herein refers to the preferential targeting by a virus (e.g., an AAV) of cells of a particular host species or of particular cell types within a host species. For example, a virus that can infect cells of the heart, lung, liver, and muscle has a broader (i.e., increased) tropism relative to a virus that can infect only lung and muscle cells, Tropism can also include the dependence of a virus on particular types of cell surface molecules of the host. For example, some viruses can infect only cells with surface glycosaminoglycans, while other viruses can infect only cells with sialic acid (such dependencies can be tested using various cells lines deficient in particular classes of molecules as potential host cells for viral infection). In some cases, the tropism of a virus describes the virus's relative preferences. For example, a first virus may be able to infect all cell types but is much more successful in infecting those cells with surface glycosaminoglycans. A second virus can be considered to have a similar (or identical) tropism as the first virus if the second virus also prefers the same characteristics (e.g., the second virus is also more successful in infecting those cells with surface glycosaminoglycans), even if the absolute transduction efficiencies are not similar. For example, the second virus might be more efficient than the first virus at infecting every given cell type tested, but if the relative preferences are similar (or identical), the second virus can still be considered to have a similar (or identical) tropism as the first virus. In some embodiments, the tropism of a virion comprising a subject variant AAV capsid protein is not altered relative to a naturally occurring virion. In some embodiments, the tropism of a virion comprising a subject variant AAV capsid protein is expanded (i.e., broadened) relative to a naturally occurring virion. In some embodiments, the tropism of a virion comprising a subject variant AAV capsid protein is reduced relative to a naturally occurring virion.

The terminology “replication-competent” virus (e.g., a replication-competent AAV) refers to a phenotypically wild-type virus that is infectious, and is also capable of being replicated in an infected cell (i.e. in the presence of a helper virus or helper virus functions). In the case of AAV, replication competence generally requires the presence of functional AAV packaging genes. In general, rAAV vectors as described herein are replication-incompetent in mammalian cells (especially in human cells) by virtue of the lack of one or more AAV packaging genes. Typically, such rAAV vectors lack any AAV packaging gene sequences in order to minimize the possibility that replication competent AAV are generated by recombination between AAV packaging genes and an incoming rAAV vector. In many embodiments, rAAV vector preparations as described herein are those which contain few if any replication competent AAV (rcAAV, also referred to as RCA) (e.g., less than about 1 rcAAV per 10 rAAV particles, less than about 1 rcAAV per 10 rAAV particles, less than about 1 rcAAV per 10 rAAV particles, less than about 1 rcAAV per 10ⁿrAAV particles, or no rcAAV).

The term “polynucleotide” refers to a polymeric form of nucleotides of any length, including deoxyribonucleotides or ribonucleotides, or analogs thereof, A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment herein that comprises a polynucleotide encompasses both the double-stranded form and each of two complementary single-stranded forms known or predicted to make up the double-stranded form.

A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the world wide web at nebi.nlm.nih.gov/BLAST/. Another alignment algorithm is PASTA, available in the Genetics Computing Group (GCC) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70:173-187 (1997). Also, the GAP program using the Needleman and Wunsch alignment method can be utilized to align sequences, See J. Mol. Biol. 48:443-453 (1970).

The term “gene” refers to a polynucleotide that performs a function of some kind in the cell. For example, a gene can contain an open reading frame that is capable of encoding a gene product. One example of a gene product is a protein, which is transcribed and translated from the gene. Another example of a gene product is an RNA, e.g., a functional RNA product, e.g., an aptamer, an interfering RNA, a ribosomal RNA (rRNA), a transfer RNA (tRNA), a non-coding RNA (ncRNA), a guide RNA for nucleases, etc., which is transcribed but not translated.

The terminology “gene expression product” or “gene product” is a molecule resulting from expression of a particular gene, as defined above. Gene expression products include, e.g., a polypeptide, an aptamer, an interfering RNA, a messenger RNA (mRNA), an rRNA, a tRNA, a non-coding RNA (ncRNA), and the like.

The term “siRNA agent” (“small interfering” or “short interfering RNA” (or siRNA)) is an RNA duplex of nucleotides that is targeted to a gene interest (a “target gene”). An “RNA duplex” refers to the structure termed by the complementary pairing between two regions of a RNA molecule, forming a region of double stranded. RNA (dsRNA). siRNA is “targeted” to a gene in that the nucleotide sequence of the duplex portion of the siRNA is complementary to a nucleotide sequence of the targeted gene. In some embodiments, the length of the duplex of siRNAs is less than 30 nucleotides. In some embodiments, the duplex can be 29, 28, 27, 26, 25, 24, 23, 22, 21, 20, 19, 18, 17, 16, 15, 14, 13, 12, 11 or 10 nucleotides in tenth. In some embodiments, the length of the duplex is 19-25 nucleotides in length. In some embodiments. “siRNA-mediated gene targeting is accomplished through the use of DNA-directed RNA interference (ddRNAI) which is a gene-silencing technique that utilizes DNA constructs to activate an animal cell's endogenous RNA interference (RNAi) pathways. Such DNA constructs are designed to express self-complementary double-stranded RNAs, typically short-hairpin RNAs (shRNA), that once processed bring about silencing of a target gene or genes. Any RNA, including endogenous mRNAs or viral RNAs, can be silenced by designing constructs to express double-stranded RNA complementary to the desired mRNA target. As such, the RNA duplex portion of an siRNA agent can be part of a short hairpin structure referred to as shRNA. In addition to the duplex portion, the hairpin structure may contain a loop portion positioned between the two sequences that form the duplex. The loop can vary in length. In some embodiments the loop is 5, 6, 7, 8, 9, 10, 11, 12 or 13 nucleotides in length. The hairpin structure can also contain 3′ or 5′ overhang portions. In some embodiments, the overhang is a 3′ or a 5′ overhang 0, 1, 2, 3, 4 or 5 nucleotides in length. In general, the level of expression product (e.g., mRNA, polypeptide, etc.) of a target gene is reduced by an siRNA agent (e.g., an siRNA, an shRNA, etc.) that contains specific double stranded nucleotide sequences that are complementary to at least a 19-25 nucleotide long segment (e.g., a 2021 nucleotide sequence) of the target gene transcript, including the 5′ untranslated (UT) region, the ORF, or the 3′ UT region. In some embodiments, short interfering RNAs are about 19-25 nt in length. See, e.g., PCT applications WO0144895. WO99/32619. WO01/75164. WO01/92513. WO01/29058. WO01/89304. WO02/16620, and WO02/29858; and U.S. Patent Publication No. 20040023390 for descriptions of siRNA technology. The siRNA and/or shRNA can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal.

The terminology “antisense RNA” encompasses RNA that is complementary to a gene expression product. For example, an antisense RNA targeted to a specific mRNA is an RNA-based agent (or can be a modified RNA) that is complementary to the mRNA, where hybridization of the antisense RNA to the mRNA alters the expression of the mRNA (e.g., via altering the stability of the RNA, altering the translation of the RNA, etc.). Also included in “antisense RNA” are nucleic acids encoding an antisense RNA.

With regards to “CRISPR/Cas9 agents”, the term “CRISPR” encompasses Clustered regularly interspaced short palindromic repeats/CRISPR-associated (Cas) systems that evolved to provide bacteria and archaea with adaptive immunity against viruses and plasmids by using CRISPR RNAs (crRNAs) to guide the silencing of invading nucleic acids. The Cas9 protein (or functional equivalent and/or variant thereof, i.e., Cas9)-like protein) naturally contains DNA endonuclease activity that depends on association of the protein with two naturally occurring or synthetic RNA molecules called crRNA and tracrRNA (also called guide RNAs). In some cases, the two molecules are covalently linked to form a single molecule (also called a single guide RNA (“sgRNA”)). Thus, the Cas9 or Cas9-like protein associates with a DNA-targeting RNA (which term encompasses both the two-molecule guide RNA configuration and the single-molecule guide RNA configuration), which activates the Cas9 or Cas9-like protein and guides the protein to a target nucleic acid sequence.

If the Cas9 or Cas9-like protein retains its natural enzymatic function, it will cleave target DNA to create a double-strand break, which can lead to genome alteration (i.e., editing: deletion, insertion (when a donor polynucleotide is present), replacement, etc.), thereby altering gene expression. Some variants of Cas9 (which variants are encompassed by the term Cas9-like) have been altered such that they have a decreased DNA cleaving activity (in some cases, they cleave a single strand instead of both strands of the target DNA, while in other cases, they have severely reduced to no DNA cleavage activity). Cas9-like proteins with decreased DNA-cleavage activity (even no DNA-cleaving activity) can still be guided to a target DNA to block RNA polymerase activity. Alternatively, the Cas9 or Cas9-like protein may be modified by fusing a VP64 transcription activation domain to the Cas9 protein and codelivering the fusion protein with a MS2-P65-HSF1 helper protein and a single guide RNA comprising MS2 RNA aptamers at the tetraloop and stem-loop to form a Synergistic Activation Mediator (Cas9-SAM) complex in the cell that activates transcription. Thus enzymatically inactive Cas9-like proteins can be targeted to a specific location in a target DNA by a DNA-targeting RNA in order to block or activate transcription of the target DNA. The term “CRISPR′Cas9 agents” as used herein encompasses all forms of CRISPR/Cas9 as described above or as known in the art.

Detailed information regarding CRISPR agents can be found, for example in (a) Jinek et. al., Science. 2012 Aug. 17: 337 (6096): 816-21: “A programmable dual-RNA-guided DNA endonuclease in adaptive bacterial immunity”: (b) Qi et al., Cell. 2013 Feb. 28: 152 (5): 1173-83: “Repurposing CRISPR, as an RNA-guided platform for sequence-specific control of gene expression”, and (c) U.S. patent application Ser. No. 13/842,859 and PCT application number PCT/US13/32589; all of which are hereby incorporated by reference in their entirety. Thus, the term “CRISPR agent” as used herein encompasses any agent (or nucleic acid encoding such an agent), comprising naturally occurring and/or synthetic sequences, that can be used in the Cas9-based system (e.g., a Cas) or Cas9-like protein: any component of a DNA-targeting RNA, e.g., a crRNA-like RNA, a tracrRNA-like RNA, a single guide RNA, etc.: a donor polynucleotide; and the like).

By “Zinc-finger nucleases” (ZFNs) it is meant artificial DNA endonucleases generated by fusing a zinc finger DNA binding domain to a DNA cleavage domain. ZFNs can be engineered to target desired DNA sequences and this enables zinc-finger nucleases to cleave unique target sequences. When introduced into a cell. ZFNs can be used to edit target DNA in the cell (e.g., the cell's genome) by inducing double strand breaks. For more information on the use of ZFNs, see, for example: Asuri et al., Mol Ther. 2012 February; 20 (2): 329-38: Bibikova et al. Science. 2003 May 2: 300 (5620): 764: Wood et al. Science. 2011 Jul. 15: 333 (6040): 307; Ochiai et al. Genes Cells. 2010 August; 15 (8): 875-85; Takasu et. al., Insect Biochem Mol 2010 October; 40 (10): 759-65; Ekker et al, Zebrafish 2008 Summer: 5 (2): 121-3: Young et al. Proc Natl Acad Sci USA. 2011 Apr. 26: 108 (17): 7052-7: Goldberg et al, Cell. 2010 Mar. 5: 140 (5): 678-91; Geurts et al. Science. 2009 Jul. 24; 325 (5939): 433: Flisikowska et al, PLOS One. 2011: 6 (6): e21045. doi: 10.1371/journal.pone.0021045. Epub 2011 Jun. 13: Flauschild et al, Proc Natl Acad Sci U S A. 2011 Jul. 19: 108 (29): 12013-7; and Yu et al, Cell Res. 2011 November; 21 (11): 1638-40: all of which are herein incorporated by reference for their teachings related to ZFNs. The term “ZFN agent” encompasses a zinc finger nuclease and/or a polynucleotide comprising a nucleotide sequence encoding a zinc finger nuclease.

The terminology “Transcription activator-like effector nuclease” or “TALEN” agents refers to Transcription activator-like effector nucleases (TALENs) are artificial DNA endonucleases generated by fusing a TAL (Transcription activator-like) effector DNA binding domain to a DNA cleavage domain. TALENS can be quickly engineered to bind practically any desired DNA sequence and when introduced into a cell, TALENs can be used to edit target DNA in the cell (e.g., the cell's genome) by inducing double strand breaks. For more information on the use of TALENs, see, for example: Hockemeyer et al. Nat Biotechnol. 2011 Jul. 7: 29 (8): 731-4; Wood et al. Science. 2011 Jul. 15: 333 (6040): 307: Tesson et al. Nat Biotechnol, 2011 Aug. 5: 29 (8): 695-6; and Huang et. al., Nat Biotechnol. 2011 Aug. 5: 29 (8): 699-700; all of which are herein incorporated by reference for their teachings related to TALENs. The term “TALEN agent” encompasses a TALEN and/or a polynucleotide comprising a nucleotide sequence encoding a TALEN.

The terminology “control element” or “control sequence” refers to a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. Promoters may be ubiquitously acting, i.e., active in many cell types, e.g., CAG or CMV promoters; or tissue or cell specific.

The terminology “operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

The terminology “expression vector” encompasses a vector comprising a polynucleotide region which encodes a polypeptide of interest, and is used for effecting the expression of the protein in an intended target cell. An expression vector may also comprise control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

The term “heterologous” means derived from a genotypically distinct entity from that of the rest of the entity to which it is being compared. For example, a polynucleotide introduced by genetic engineering techniques into a plasmid or vector derived from a different species is a heterologous polynucleotide. A promoter removed from its native coding sequence and operatively linked to a coding sequence with which it is not naturally found linked is a heterologous promoter. Thus, for example, an rAAV that includes a heterologous nucleic acid sequence encoding a heterologous gene product is an rAAV that includes a polynucleotide not normally included in a naturally-occurring, wild-type AAV, and the encoded heterologous gene product is a gene product not normally encoded by a naturally-occurring, wild type AAV.

The terminology “genetic alteration” and “genetic modification” (and grammatical variants thereof), are used interchangeably herein to refer to a process wherein a genetic element (e.g., a polynucleotide) is introduced into a cell other than by mitosis or meiosis. The element may be heterologous to the cell, or it may be an additional copy or improved version of an element already present in the cell. Genetic alteration may be effected, for example, by transfecting a cell with a recombinant plasmid or other polynucleotide through any process known in the art, such as electroporation, calcium phosphate precipitation, or contacting with a polynucleotide-liposome complex. Genetic alteration may also be effected, for example, by transduction or infection with a DNA or RNA virus or viral vector. Generally, the genetic element is introduced into a chromosome or mini-chromosome in the cell: but any alteration that changes the phenotype and/or genotype of the cell and its progeny is included in this term.

With regards to cell modification, the terminology “genetically modified” or “transformed” or “transfected” or “transduced” by exogenous DNA (e.g., via a recombinant virus) refers to when such DNA has been introduced inside the cell. The presence of the exogenous DNA results in permanent or transient genetic change. The transforming DNA may or may not be integrated (covalently linked) into the genome of the cell. A “clone” is a population of cells derived from a single cell or common ancestor by mitosis. A “cell line” is a clone of a primary cell that is capable of stable growth in vitro for many generations.

As used herein, a cell is said to be “stably” altered, transduced, genetically modified, or transformed with a genetic sequence if the sequence is available to perform its function during extended culture of the cell in vitro and/or for an extended period of time in vivo. Generally, such a cell is “heritably” altered (genetically modified) in that a genetic alteration is introduced which is also inheritable by progeny of the altered cell.

The terms “polypeptide,” “peptide.” and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides such as anti-angiogenic polypeptides, neuroprotective polypeptides, and the like, when discussed in the context of delivering a gene product to a mammalian subject, and compositions therefor, refer to the respective intact polypeptide, or any fragment or genetically engineered derivative thereof, which retains the desired biochemical function of the intact protein. Similarly, references to nucleic acids encoding anti-angiogenic polypeptides, nucleic acids encoding neuroprotective polypeptides, and other such nucleic acids for use in delivery of a gene product to a mammalian subject (which may be referred to as “transgenes” to be delivered to a recipient cell), include polynucleotides encoding the intact polypeptide or any fragment or genetically engineered derivative possessing the desired biochemical function.

As used herein, an “isolated” plasmid, nucleic acid, vector, virus, virion, host cell, protein, or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Enrichment can be measured on an absolute basis, such as weight per volume of solution, or it can be measured in relation to a second, potentially interfering substance present in the source mixture. Increasing enrichments of the embodiments of this disclosure are increasingly more isolated. An isolated plasmid, nucleic acid, vector, virus, host cell, or other substance is in some embodiments purified, e.g., from about 80% to about 90% pure, at least about 90% pure, at least about 95% pure, at least about 98% pure, or at least about 99%, or more, pure.

As used herein, the terms “treatment.” “treating.” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease (and/or symptoms caused by the disease) from occurring in a subject which may be predisposed to the disease or at risk of acquiring the disease but has not yet been diagnosed as having it: (b) inhibiting the disease (and/or symptoms caused by the disease), i.e., arresting its development; and (c) relieving the disease (and/or symptoms caused by the disease), i.e., causing regression of the disease (and/or symptoms caused by the disease), i.e., ameliorating the disease and/or one or more symptoms of the disease. For example, the subject compositions and methods may be directed towards the treatment of lung disease.

The terms “individual,” “host,” “subject,” and “patient” are used interchangeably herein, and refer to a mammal, including, but not limited to, humans: non-human primates, including simians; mammalian sport animals (e.g., horses); mammalian farm animals (e.g., sheep, goats, etc.); mammalian pets (dogs, cats, etc.); and rodents (e.g., mice, rats, etc.).

In some embodiments, the individual is a human who has previously been naturally exposed to AAV and as a result harbors anti-AAV antibodies (i.e., AAV neutralizing antibodies). In some embodiments, the individual is a human who has previously been administered an AAV vector (and as a result may harbor anti-AAV antibodies) and needs re-administration of vector for treatment of a different condition or for further treatment of the same condition. Based on positive results in clinical trials involving AAV gene delivery to, for example, liver, muscle, and retina—all tissues affected by neutralizing antibodies against this vehicle—there are many such therapeutic applications/disease targets.

The term “effective amount” as used herein is an amount sufficient to effect beneficial or desired clinical results. An effective amount can be administered in one or more administrations. For purposes of this disclosure, an effective amount of a compound (e.g., an infectious rAAV virion) is an amount that is sufficient to palliate, ameliorate, stabilize, reverse, prevent, slow or delay the progression of (and/or symptoms associated with) a particular disease state (e.g., a lung disease). Accordingly, an effective amount of an infectious rAAV virion is an amount of the infectious rAAV virion that is able to effectively deliver a heterologous nucleic acid to a target cell (or target cells) of the individual. Effective amounts may be determined preclinically by, e.g., detecting in the cell or tissue the gene product (RNA, protein) that is encoded by the heterologous nucleic acid sequence using techniques that are well understood in the art, e.g., RT-PCR, western blotting, ELISA, fluorescence or other reporter readouts, and the like. Effective amounts may be determined clinically by, e.g., detecting a change in the onset or progression of disease using methods known in the art.

The terminology “directed evolution” refers to a capsid engineering methodology, in vitro and/or in vivo, which emulates natural evolution through iterative rounds of genetic diversification and selection processes, thereby accumulating beneficial mutations that progressively improve the function of a biomolecule. Directed evolution often involves an in vivo method referred to as “biopanning” for selection of AAV variants from a library which variants possess a more efficient level of infectivity of a cell or tissue type of interest.

The term “interfering RNA” encompasses both small interfering RNAs and microRNAs (miRNAs) including artificial miRNAs.

A “2A peptide” refers to “self-cleaving” peptides of about 20 amino acids that produce equimolar levels of multiple genes from the same mRNA and may be used in place of IRES elements in multicistronic vectors. Non-limiting examples include T2A, P2A, E2A and F2A peptides sequences. In embodiments wherein a heterologous nucleic acid comprises nucleotide sequence encoding multiple gene products, expression of the multiple (e.g., 2) gene products can be mediated by multiple (e.g., 2) independent promoters or may be mediated by a single promoter, with the multiple transgenes separated by an internal ribosome entry site (IRES) or a 2A peptide sequence.

By “increased resistance” it is meant that a subject infectious rAAV virion exhibits an increased infectivity in the presence of human anti-AAV antibodies. Viral infectivity can be expressed as the ratio of infectious viral particles to total viral particles. Thus in increased infectivity means an increased ratio of infectious viral particles to total viral particles. To determine resistance of an AAV to human anti-AAV antibodies, infectivity of the AAV is measured in the presence of various concentrations of human anti-AAV antibodies in order to obtain the antibody concentration (e.g., serum concentration, IVIG concentration, etc.) (mg/mL) required to reduce gene delivery efficiency (i.e., infectivity) to 50% of that in the absence of human anti-AAV antibodies. A virus that requires a higher antibody concentration to reduce gene delivery efficiency to 50% of that in the absence of human anti-AAV antibodies is said to have increased resistance to antibody neutralization. Thus, a two-fold increase in resistance means a two-fold increase in the antibody concentration required to reduce gene delivery efficiency to 50% of that in the absence of human anti-AAV antibodies. In some embodiments, a subject infectious rAAV virion exhibits at least about 1.5-fold (e.g., at least about 1.5-fold, at least about 2-fold, at least about 3-fold, at least about 4-fold, at least about 5-fold, at least about 7.5-fold, at least about 10-fold, at least about 12-fold, at least about 15-fold, at least about 17-fold, at least about 20-fold, at least about 25-fold, at least about 30-fold, at least about 40-fold, at least about 50-fold, at least about 75-fold, at least about 100-fold, at least about 150-fold, at least about 200-fold, at least about 250-fold, at least about 300-fold, etc.) greater resistance to human AAV neutralizing antibodies than the resistance exhibited by a wild type AAV (e.g., AAV2 (wild type AAV serotype 2)) or an AAV comprising a wild-type capsid protein.

A subject infectious rAAV virion can be said to exhibit increased transduction of mammalian cells in the presence of human AAV neutralizing antibodies. In some embodiments, a subject infectious rAAV virion exhibits at least about 1.5-fold (e.g., at least about 1.5-fold, at least about 2-fold, at least about 3-fold, at least about 4-fold, at least about 5-fold, at least about 7.5-fold, at least about 10-fold, at least about 12-fold, at least about 15-fold, at least about 17-fold, at least about 20-fold, at least about 25-fold, at least about 30-fold, at least about 40-fold, at least about 50-fold, at least about 75-fold, at least about 100-fold, at least about 150-fold, at least about 200-fold, at least about 250-fold, at least about 300-fold, etc.) greater transduction of mammalian cells in the presence of human AAV neutralizing antibodies than the transduction exhibited by a wild type AAV (e.g., AAV2 (wild type AAV serotype 2)) or an AAV comprising a wild-type capsid protein.

In some embodiments, a subject infectious rAAV virion exhibits decreased binding to a neutralizing antibody that binds a wild-type AAV capsid protein. For example, a subject infectious rAAV virion can exhibit at least about 1.5-fold (e.g., at least about 1.5-fold, at least about 2-fold, at least about 3-fold, at least about 4-fold, at least about 5-fold, at least about 7.5-fold, at least about 10-fold, at least about 12-fold, at least about 15-fold, at least about 17-fold, at least about 20-fold, at least about 25-fold, at least about 30-fold, at least about 40-fold, at least about 50-fold, at least about 75-fold, at least about 100-fold, at least about 150-fold, at least about 200-fold, at least about 250-fold, at least about 300-fold, etc.) reduced binding (e.g., reduced affinity) to a neutralizing antibody that binds a wild-type capsid AAV protein, compared to the binding affinity of the antibody to wild-type AAV capsid protein.

In some embodiments, an anti-AAV neutralizing antibody binds to a subject infectious rAAV virion with an affinity of less than about 10⁻⁷M, less than about 5×10⁻⁶ M, less than about 10⁻⁶M, less than about 5×10⁻⁵M, less than about 10⁻⁵ M, less than about 10⁻⁴ M, or lower.

Adeno-associated viruses (AAVs) are a family of parvoviruses with a 4.7 kb single-stranded DNA genome contained inside a non-enveloped capsid. The viral genome of a naturally occurring AAV has 2 inverted terminal repeats (ITR)—which function as the viral origin of replication and packaging signal—flanking 2 primary open reading frames (ORF): rep (encoding proteins that function in viral replication, transcriptional regulation, site-specific integration, and virion assembly) and cap. The cap ORF codes for 3 structural proteins that assemble to form a 60-mer viral capsid. Many naturally occurring AAV variants and serotypes have been isolated, and none have been associated with human disease.

Recombinant versions of AAV can be used as gene delivery vectors, where a marker or therapeutic gene of interest is inserted between the ITRs in place of rep and cap. These vectors have been shown to transduce both dividing and non-dividing cells in vitro and in vivo and can result in stable transgene expression for years in post-mitotic tissue. See e.g., Knipe D M, Howley P M, Fields' Virology. Lippincott Williams & Wilkins, Philadelphia, Pa., USA, 2007; Gao G-P, Alvira M R, Wang L, Calcedo R, Johnston J, Wilson S M. Novel adeno-associated viruses from rhesus monkeys as vectors for human gene therapy. Proc Natl Acad Sci USA 2002; 99:11854-9; Atchison R W, Casty B C, Hammon W M. Adenovirus-Associated Defective Virus Particles, Science 1965:149: 754-6; Hoggan M D, Blacklow NR. Rowe W P. Studies of small DNA viruses found in various adenovirus preparations: physical, biological, and immunological characteristics, Proc Natl Acad USA 1966:55: 1467-74; Blacklow N R, Hoggan M D, Rowe W P. Isolation of adenovirus-associated viruses from man. Proc Natl Acad Sci USA 1967:58: 1410-5; Bantel-Schaal U, zur Hauser” H. Characterization of the DNA of a defective human parvovirus isolated from a genital site. Virology 1984:134: 52-63; Mayor H D, Melnick J L. Small deoxyribonucleic acid-containing viruses (picodnavirus group). Nature 1966; 210:331-2; Mori S, Wang L, Takeuchi T, Kanda T. Two novel adeno-associated viruses from cynomolgus monkey: pseudotyping characterization of capsid protein. Virology 2004:330: 375-83; Flotte T R. Gene therapy progress and prospects: recombinant adeno-associated virus (rAAV) vectors. Gene Ther 2004:11: 805-10.

Recombinant AAV (referred to herein interchangeably as “AAV” or “TAAV”) has yielded promising results in an increasing number of clinical trials. However, there are impediments to gene delivery that may limit AAV's utility, such as anti-capsid immune responses, low transduction of certain tissues, an inability for targeted delivery to specific cell types and a relatively low carrying capacity. In many situations, there is insufficient mechanistic knowledge to effectively empower rational design with the capacity to improve AAV. As an alternative, directed evolution has emerged as a strategy to create novel AAV variants that meet specific biomedical needs. Directed evolution strategies harness genetic diversification and selection processes to enable the accumulation of beneficial mutations that progressively improve the function of a biomolecule. In this process, wild-type AAV cap genes are diversified by several approaches to create large genetic libraries that are packaged to generate libraries of viral particles, and selective pressure is then applied to isolate novel variants that can overcome gene delivery barriers. Importantly, the mechanistic basis underlying a gene delivery problem does not need to be known for directed evolution of function, which can thus accelerate the development of enhanced vectors.

Typically, the variants disclosed herein were generated through use of an AAV library and/or libraries. Such an AAV library or libraries is/are generated by mutating the cop gene, the gene which encodes the structural proteins of the AAV capsid, by a range of directed evolution techniques known by and readily available to the skilled artisan in the field of viral genome engineering. See e.g., Bartel et al. Am. Soc. Gene Cell Then. 15^{th} Annu. Meet. 20, 5140 (2012): Bowles, D. et al. J. Virol. 77, 423-432 (2003): Gray et al. Mol. Ther. 18, 570-578 (2010): Grimm. D. et al. J. Virol. 82, 5887-5911; Koerber. J. T. et al. Mol. Then. 16, 1703-1709 (2008): Li W. et al. Mol. Ther. 16, 1252-1260 (2008); Koerber. J. T. et al. Methods Mol. Biol, 434, 161-170 (2008); Koerber, J. T. et al. Hum. Gene Then. 18, 367-378 (2007); and Koerber, J. T. et al. Mol. Ther. 17, 2088-2095 (2009). Such techniques, without limitation, are as follows: i) Error-prone PCR to introduce random point mutations into the AAV cap open reading frame (ORF) at a predetermined, modifiable rate; ii) In vitro or in vivo viral recombination or “DNA shuffling” to generate random chimeras of AAV cap genes to yield a gene library with multiple AAV serotypes; iii) Random peptide insertions at defined sites of the capsid by ligation of degenerate oligonucleotides in the cap ORF; iv) Defined insertions of peptide-encoding sequences into random locations of the AAV cap ORF using transpose mutagenesis: v) Replacing surface loops of AAV capsids with libraries of peptide sequences bioinformationally designed based on the level of conservation of each amino acid position among natural AAV serotypes and variants to generate “loop-swap” libraries; vi) Random amino acid substitution at positions of degeneracy between AAV serotypes to generate libraries of ancestral variants (Santiago-Ortiz et al., 2015); and a combination of such techniques thereof.

DNA shuffling generates chimeras which combine their parental properties in unique and, often beneficial, ways; however, some may be incapable of packaging which, in effect, reduces the diversity of the library. Diversity concentration of the library is achieved through peptide insertion techniques such as, without limitation, iii-iv) above. Diversity of the library is also concentrated in techniques such as v) above, and such concentration is directed onto multiple hypervariable regions, which lie on surface exposed loops, of the AAV capsid. While many of the techniques generate variant capsids with only a small area of the capsid mutated, these techniques can be paired with additional mutagenesis strategies to modify the full capsid.

Once the AAV library or libraries is/are generated, viruses are then packaged, such that each AAV particle is comprised of a mutant capsid surrounding a cap gene encoding that capsid, and purified. Variants of the library are then subjected to in vitro and/or in vivo selective pressure techniques known by and readily available to the skilled artisan in the field of AAV. See e.g., Maheshri, N. et al. Nature Biotech, 24, 198-204 (2006): Dalkara. D. et al. Sci. Tran.sl. Med. 5, 189ra76 (2013): Lisowski, L. et al. Nature. 506, 382-286 (2013): Yang, L. et al. IPNAS, 106, 3946-3951 (2009); Gao, G. et al. Mol. Them. 13, 77-87 (2006); and Bell, P. et al. Hum. Gene. Ther. 22, 985-997 (2011). For example, without limitation, AAV variants can be selected using i) affinity columns in which elution of different fractions yields variants with altered binding properties; ii) primary cells-isolated from tissue samples or immortal cells lines that mimic the behavior of cells in the human body—which yield AAV variants with increased efficiency and/or tissue specificity; iii) animal models—which mimic a clinical gene therapy environment—which yield AAV variants that have successfully infected target tissue; iv) human xenograft models which yield AAV variants that have infected grafted human cells; and/or a combination of selection techniques thereof.

Once viruses are selected, they may be recovered by known techniques such as, without limitation, adenovirus-mediated replication, PCR amplification, Next Generation sequencing and cloning, and the like, Virus clones are then enriched through repeated rounds of the selection techniques and AAV DNA is isolated to recover selected variant cap genes of interest. Such selected variants can be subjected to further modification or mutation and as such serve as a new starting point for further selection steps to iteratively increase AAV viral fitness. However, in certain instances, successful capsids have been generated without additional mutation.

The AAV variants disclosed herein were generated at least in part through the use of in vivo directed evolution methodology, such as the techniques described above, involving the use of primate lung screens following aerosol administration. As such, the AAV variant capsids disclosed herein comprise one or more modifications in amino acid sequence that confer more efficient transduction of primate lung cells than a corresponding parental AAV capsid protein. As used herein, a “corresponding parental AAV capsid protein” refers to an AAV capsid protein of the same wild-type or variant AAV serotype as the subject variant AAV capsid protein but that does not comprise the one or more amino acid sequence modifications of the subject variant AAV capsid protein.

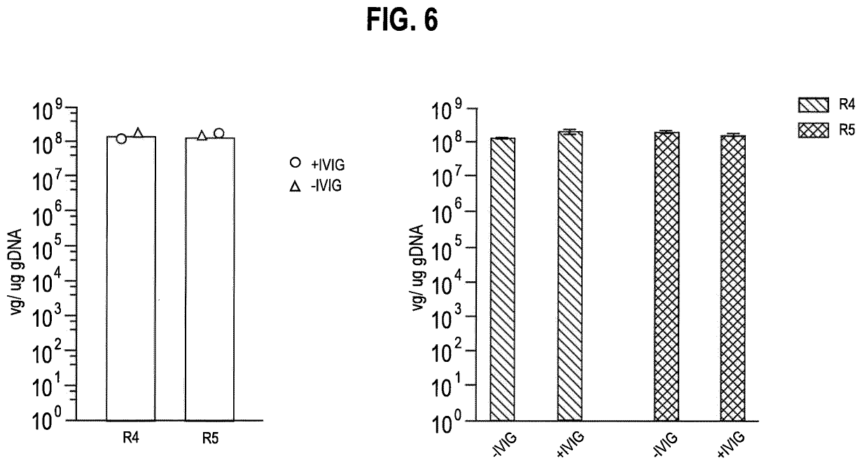

In some embodiments, the subject variant AAV capsid protein comprises a heterologous peptide of from about 5 amino acids to about 20 amino acids inserted by covalent linkage into an AAV capsid protein GH loop, or loop IV, relative to a corresponding parental AAV capsid protein. By the “GH loop,” or loop IV, of the AAV capsid protein it is meant the solvent-accessible portion referred to in the art as the OH loop, or loop IV, of AAV capsid protein. For the OH loop/loop IV of AAV capsid, see, e.g., van Viet et al. (2006) Mol. Ther. 14:809; Padron et al. (2005) J. Virol. 79:5047; and Shen et al. (2007) Mol. Ther. 15:1955, Thus, for example, the insertion site can be within about amino acids 411-650 of an AAV VP1 capsid protein. For example, the insertion site can be within amino acids 571-612 of AAV1 VP1, amino acids 570-611 of AAV2 VP1, within amino acids 571-612 of AAV3A VP1, within amino acids 571-612 of AAV3B VP1, within amino acids 569-610 of AAV4 VP1, within amino acids 560-601 of AAV5 VP1, within amino acids 571 to 612 of AAV6 VP1, within amino acids 572 to 613 of AAV7 VP1, within amino acids 573 to 614 of AAV8 VP1, within amino acids 571 to 612 of AAV9 VP1, or within amino acids 573 to 614 of AAV10 VP1, or the corresponding amino acids of any variant thereof, Those skilled in the art would know, based on a comparison of the amino acid sequences of capsid proteins of various AAV serotypes, where an insertion site “corresponding to amino acids of AAV2” would be in a capsid protein of any given AAV serotype. See also FIG. 6 of U.S. Application Publication No 2019/0255192 for an alignment of wild-type AAV SEQ ID NOS: 1-11 which provides amino acid locations between and across the wild-type (naturally occurring) serotypes AAV1, AAV2. AAV3A, AAV3B, and AAV4-10, the entire contents of which are incorporated herein by reference.

In certain embodiments, the insertion site is a single insertion site between two adjacent amino acids located between amino acids 570-614 of VP1 of any wild-type AAV serotype or AAV variant, e.g., the insertion site is between two adjacent amino acids located in amino acids 570-610, amino acids 580-600, amino acids 570-575, amino acids 575-580, amino acids 5809585, amino acids 585-590, amino acids 590-600, or amino acids 600-614, of VP1 of any AAV serotype or variant. For example, the insertion site can be between amino acids 580 and 581, amino acids 581 and 582, amino acids 583 and 584, amino acids 584 and 585, amino acids 585 and 586, amino acids 586 and 587, amino acids 587 and 588, amino acids 588 and 589, or amino acids 589 and 590. The insertion site can be between amino acids 575 and 576, amino acids 576 and 577, amino acids 577 and 578, amino acids 578 and 579, or amino acids 579 and 580. The insertion site can be between amino acids 590 and 591, amino acids 591 and 592, amino acids 592 and 593, amino acids 593 and 594, amino acids 594 and 595, amino acids 595 and 596, amino acids 596 and 597, amino acids 597 and 598, amino acids 598 and 599, or amino acids 599 and 600. For example, the insertion site can be between amino acids 587 and 588 of AAV2, between amino acids 590 and 591 of AAV1, between amino acids 588 and 589 of AAV3A, between amino acids 588 and 589 of AAV3B, between amino acids 584 and 585 of AAV4, between amino acids 575 and 576 of AAV5, between amino acids 590 and 591 of AAV6, between amino acids 589 and 590 of AAV7, between amino acids 590 and 591 of AAV8, between amino acids 588 and 589 of AAV9), or between amino acids 588 and 589 of AAV10. In certain embodiments, the insertion site is between amino acids 587 and 588 of AAV2 or is between amino acids 588 and 589 of AAV2.

In some embodiments, a peptide insertion disclosed herein has a length of 5 amino acids, 6 amino acids, 7 amino acids, 8 amino acids, 9 amino acids, 10 amino acids, 11 amino acids, 12 amino acids. 13 amino acids, 14 amino acids, 15 amino acids, 16 amino acids, 17 amino acids, 18 amino acids, 19 amino acids, or 20 amino acids. In another embodiment, a peptide insertion disclosed herein comprises from 1 to 4 spacer amino acids at the amino terminus (N-terminus) and/or at the carboxyl terminus (C-terminus) of any one of the peptide insertions disclosed herein. Exemplary spacer amino acids include, without limitation, leucine (L), alanine (A), glycine (G), serine(S), threonine (T), and proline (P). In certain embodiments, a peptide insertion comprises 2 spacer amino acids at the N-terminus and 2 spacer amino acids at the C-terminus. In other embodiments, a peptide insertion comprises 2 spacer amino acids at the N-terminus and 1 spacer amino acids at the C-terminus.

In some embodiments, the insertion peptide comprises, consists essentially of, or consists of an amino acid sequence selected from HDITKNI (SEQ ID NO: 12), NQDYTKT (SEQ ID NO:13), DNTVTRS (SEQ ID NO:14), SNSVQSI (SEQ ID NO:15), NSTRHTD (SEQ ID NO: 16), TNRTSPD (SEQ ID NO:17), ISDQTKH (SEQ ID NO:18), QADTTKN (SEQ ID NO: 19), NAVKTDF (SEQ ID NO:20), TNQTLSA (SEQ ID NO:21), ENRTTSN (SEQ ID NO:22), PQQDTTH (SEQ ID NO:23), NATNHVI (SEQ ID NO:24), TNNSKPD (SEQ ID NO:25), SKLTLNN (SEQ ID NO:26), VTAGMGA (SEQ ID NO:27), PNSTTNN (SEQ ID NO:28), NSTSRID (SEQ ID NO:29), VASHTNN (SEQ ID NO:30), RSHQEIP (SEQ ID NO:31), LNTTKDI (SEQ ID NO:32), IIDATKN (SEQ ID NO:33), NHISQTN (SEQ ID NO:34), SNSAHIT (SEQ ID NO:35), STHQSNN (SEQ ID NO:36), KTPNLTS (SEQ ID NO:37), SNTPALS (SEQ ID NO:38), SPGATTN (SEQ ID NO:39).

In other preferred embodiments, the insertion peptide comprises, consists essentially of, or consists of from 1 to 3 spacer amino acids (Y₁-Y₃) at the amino and/or carboxyl terminus of an amino acid sequence selected from HDITKNI (SEQ ID NO:12), NQDYTKT (SEQ ID NO:13), DNTVTRS (SEQ ID NO:14), SNSVQSI (SEQ ID NO:15), NSTRHTD (SEQ ID NO:16), TNRTSPD (SEQ ID NO: 17), ISDQTKH (SEQ ID NO:18), QADTTKN (SEQ ID NO:19), NAVKTDF (SEQ ID NO:20), TNQTLSA (SEQ ID NO:21), ENRTTSN (SEQ ID NO:22), PQQDTTH (SEQ ID NO:23), NATNHVI (SEQ ID NO:24), TNNSKPD (SEQ ID NO:25), SKLTLNN (SEQ ID NO:26), VTAGMGA (SEQ ID NO: 27), PNSTTNN (SEQ ID NO:28), NSTSRID (SEQ ID NO:29), VASHTNN (SEQ ID NO: 30), RSHQEIP (SEQ ID NO:31), LNTTKDI (SEQ ID NO:32), IIDATKN (SEQ ID NO: 33), NHISQTN (SEQ ID NO:34), SNSAHIT (SEQ ID NO:35), STHQSNN (SEQ ID NO: 36), KTPNLTS (SEQ ID NO:37), SNTPALS (SEQ ID NO:38), SPGATTN (SEQ ID NO: 39). In certain such embodiments, the insertion peptide is selected from the group consisting of LAHDITKNIA (SEQ ID NO:40). LANQDYTKTA (SEQ ID NO:41), LADNTVTRSA (SEQ ID NO:42), LASNSVQSIA (SEQ ID NO:43), LANSTRHTDA (SEQ ID NO:44), LATNRTSPDA (SEQ ID NO:45), LAISDQTKHA (SEQ ID NO:46), LAQADTTKNA (SEQ ID NO:47), LANAVKTDFA (SEQ ID NO:48), LATNQTLSAA (SEQ ID NO:49), LAENRTTSNA (SEQ ID NO:50), LAPQQDTTHA (SEQ ID NO:51), LANATNHVIA (SEQ ID NO:52), LATNNSKPDA (SEQ ID NO:53), LASKLTLNNA (SEQ ID NO:54), LAVTAGMGAA (SEQ ID NO:55), LAPNSTTNNA (SEQ ID NO:56), LANSTSRIDA (SEQ ID NO:57), LAVASHTNNA (SEQ ID NO:58), LARSHQEIPA (SEQ ID NO:59), LALNTTKDIA (SEQ ID NO:60), LAIIDATKNA (SEQ ID NO:61), LANHISQTNA (SEQ ID NO:62), LASNSAHITA (SEQ ID NO:63), LASTHQSNNA (SEQ ID NO:64), LAKTPNLTSA (SEQ ID NO:65), LASNTPALSA (SEQ ID NO:66) and LASPGATTNA (SEQ ID NO:67).

In some embodiments, the subject variant AAV capsid protein does not include any other amino acid sequence modifications other than a peptide insertion of from about 5 amino acids to about 20 amino acids in the GH loop, or loop IV. For example, in some embodiments, the subject variant AAV capsid protein comprises, consists essentially of, or consists of a peptide insertion comprising an amino acid sequence selected from the group consisting of HDITKNI (SEQ ID NO: 12), NQDYTKT (SEQ ID NO: 13), DNTVTRS (SEQ ID NO: 14), SNSVQSI (SEQ ID NO:15), NSTRHTD (SEQ ID NO:16), TNRTSPD (SEQ ID NO: 17), ISDQTKH (SEQ ID NO:18), QADTTKN (SEQ ID NO:19), NAVKTDF (SEQ ID NO: 20), TNQTLSA (SEQ ID NO:21), ENRTTSN (SEQ ID NO:22), PQQDTTH (SEQ ID NO: 23), NATNHVI (SEQ ID NO:24), TNNSKPD (SEQ ID NO:25), SKLTLNN (SEQ ID NO: 26), VTAGMGA (SEQ ID NO:27), PNSTTNN (SEQ ID NO:28), NSTSRID (SEQ ID NO: 29), VASHTNN (SEQ ID NO:30), RSHQEIP (SEQ ID NO:31), LNTTKDI (SEQ ID NO: 32), IIDATKN (SEQ ID NO:33), NHISQTN (SEQ ID NO:34), SNSAHIT (SEQ ID NO: 35), STHQSNN (SEQ ID NO:36), KTPNLTS (SEQ ID NO:37), SNTPALS (SEQ ID NO: 38), SPGATTN (SEQ ID NO:39), LAHDITKNIA (SEQ ID NO:40), LANQDYTKTA (SEQ ID NO:41), LADNTVTRSA (SEQ ID NO:42), LASNSVQSIA (SEQ ID NO:43), LANSTRHTDA (SEQ ID NO:44), LATNRTSPDA (SEQ ID NO:45), LAISDQTKHA (SEQ ID NO:46), LAQADTTKNA (SEQ ID NO:47), LANAVKTDFA (SEQ ID NO:48), LATNQTLSAA (SEQ ID NO:49), LAENRTTSNA (SEQ ID NO:50), LAPQQDTTHA (SEQ ID NO:51), LANATNHVIA (SEQ ID NO:52), LATNNSKPDA (SEQ ID NO:53), LASKLTLNNA (SEQ ID NO:54), LAVTAGMGAA (SEQ ID NO:55), LAPNSTTNNA (SEQ ID NO:56), LANSTSRIDA (SEQ ID NO:57), LAVASHTNNA (SEQ ID NO:58), LARSHQEIPA (SEQ ID NO:59), LALNTTKDIA (SEQ ID NO:60), LAIIDATKNA (SEQ ID NO: 61), LANHISQTNA (SEQ ID NO:62), LASNSAHITA (SEQ ID NO:63), LASTHQSNNA (SEQ ID NO:64), LAKTPNLTSA (SEQ ID NO:65), LASNTPALSA (SEQ ID NO:66) and LASPGATTNA (SEQ ID NO:67), and the variant AAV capsid does not include any other amino acid substitutions, insertions, or deletions (i.e., the variant AAV capsid protein comprises said insertion and is otherwise identical to the corresponding AAV capsid protein). Put another way, the variant AAV capsid protein comprising said insertion is otherwise identical to the parental AAV capsid protein into which the peptide has been inserted. As another example, the subject variant AAV capsid protein comprises, consists essentially of, or consists of a peptide insertion having an amino acid sequence selected from HDITKNI (SEQ ID NO:12), NQDYTKT (SEQ ID NO:13), DNTVTRS (SEQ ID NO:14), SNSVQSI (SEQ ID NO:15), NSTRHTD (SEQ ID NO:16), TNRTSPD (SEQ ID NO:17), ISDQTKH (SEQ ID NO:18), QADTTKN (SEQ ID NO:19), NAVKTDF (SEQ ID NO:20), TNQTLSA (SEQ ID NO:21), ENRTTSN (SEQ ID NO:22), PQQDTTH (SEQ ID NO:23), NATNHVI (SEQ ID NO:24), TNNSKPD (SEQ ID NO:25), SKLTLNN (SEQ ID NO:26), VTAGMGA (SEQ ID NO:27), PNSTTNN (SEQ ID NO:28), NSTSRID (SEQ ID NO:29), VASHTNN (SEQ ID NO:30), RSHQEIP (SEQ ID NO:31), LNTTKDI (SEQ ID NO:32), IIDATKN (SEQ ID NO:33), NHISQTN (SEQ ID NO:34), SNSAHIT (SEQ ID NO:35), STHQSNN (SEQ ID NO:36), KTPNLTS (SEQ ID NO:37), SNTPALS (SEQ ID NO:38), SPGATTN (SEQ ID NO:39), LAHDITKNIA (SEQ ID NO: 40), LANQDYTKTA (SEQ ID NO:41), LADNTVTRSA (SEQ ID NO:42), LASNSVQSIA (SEQ ID NO:43), LANSTRHTDA (SEQ ID NO:44), LATNRTSPDA (SEQ ID NO:45), LAISDQTKHA (SEQ ID NO:46), LAQADTTKNA (SEQ ID NO:47), LANAVKTDFA (SEQ ID NO:48), LATNQTLSAA (SEQ ID NO:49), LAENRTTSNA (SEQ ID NO:50), LAPQQDTTHA (SEQ ID NO:51), LANATNHVIA (SEQ ID NO:52), LATNNSKPDA (SEQ ID NO:53), LASKLTLNNA (SEQ ID NO:54), LAVTAGMGAA (SEQ ID NO:55), LAPNSTTNNA (SEQ ID NO:56), LANSTSRIDA (SEQ ID NO:57), LAVASHTNNA (SEQ ID NO:58), LARSHQEIPA (SEQ ID NO:59), LALNTTKDIA (SEQ ID NO:60), LAIIDATKNA (SEQ ID NO:61), LANHISQTNA (SEQ ID NO:62), LASNSAHITA (SEQ ID NO:63), LASTHQSNNA (SEQ ID NO:64), LAKTPNLTSA (SEQ ID NO:65), LASNTPALSA (SEQ ID NO:66) and LASPGATTNA (SEQ ID NO:67), wherein the peptide insertion is located between amino acids 587 and 588 of the VP1 of the AAV2 capsid or the corresponding amino acids of a VP1 of another parental AAV, e.g., between amino acids 588 and 589 of VP1 of AAV1. AAV3A. AAV3B. AAV6 or AAV9, between amino acids 586 and 587 of VP1 of AAV4, between amino acids 577 and 578 of VP1 of AAV5, between amino acids 589 and 590 of VP1 of AAV7, between amino acids 590 to 591 of VP1 of AAV8 or AAV10, etc, wherein the variant AAV capsid protein sequence is otherwise identical to the corresponding parental AAV capsid protein sequence, e.g., any one of SEQ ID NOs: 1-12.

In other embodiments, the subject variant AAV capsid protein, in addition to comprising a peptide insertion, e.g., as disclosed herein or as known in the art, in the GH loop, comprises from about 1 to about 100 amino acid substitutions or deletions, e.g., 1 to about 5, from about 2 to about 4, from about 2 to about 5, from about 5 to about 10, from about 10 to about 15, from about 15 to about 20, from about 20 to about 25, from about 25-50, from about 50-100 amino acid substitutions or deletions compared to the parental AAV capsid protein. Thus, in some embodiments, a subject variant capsid protein comprises an amino acid sequence having a sequence identity of 85% or more, 90% or more, 95% or more, or 98% or more, e.g., or 99% or more identity to the corresponding parental AAV capsid. e.g., a wild type capsid protein as set forth in SEQ ID NOs: 1-12. In some embodiments, a subject variant capsid protein comprises an amino acid sequence having an amino acid sequence identity of at least 85%, at least 90%, at least 95%, at least 98% or at least 99% to the amino acid sequence of AAV2 capsid protein (SEQ ID NO:2).

In a further embodiment, the one or more amino acid substitutions are at amino acid residue(s) 1, 6, 15, 16, 18, 30, 34, 37, 38, 57, 65, 66, 81, 91, 99, 101, 103, 109, 118, 120, 133, 134, 135, 136, 137, 138, 144, 164, 176, 188, 196, 200, 213, 220, 226, 236, 240, 250, 283, 312, 344, 347, 363, 368, 371, 376, 399, 428, 436, 449, 451, 456, 463, 469, 472, 484, 491, 524, 532, 535, 551, 585, 591, 593, 594, 608, 641, 688, 698, 705, 708, 719, 721, and/or 735 of AAV2 VP1 capsid protein as numbered prior to insertion of the peptide, or the corresponding amino acid residue(s) of another AAV capsid protein. In some such embodiments, the one or more amino acid substitutions are selected from the group consisting of Y6F, S16Y, G18E, P30L, R37L, H38Q, V65A, L911, E99D, R103L, R103C, S109T, V118A, Q120H, E133D, E134Q, P135A, V136G, K137E, T138R, T200I, D213Y, G220R, P250S, D283E, N312K, T344S, E347D, G376A, P399H, G406E, Q428H, P436H, N449D, P451Q, N469D, D472N, T491I, K532E, K544E, R585K, A591D, A593E, D594N, D608N, H641N, K688R, N705S, and V708I of AAV2 VP1 capsid protein as numbered prior to the insertion of the peptide, or the corresponding amino acid residue(s) of another AAV capsid protein. In other such embodiments, the one or more amino acid substitutions are selected from the group consisting of M1L, L15P, P34A, N57D, N66K, R81Q, Q101R, S109T, R144K, R144M, Q164K, T176P, L188I, S196Y, G226E, G236V, 1240T, P250S, N312K, P363L, D368H, N449D, 1456K, S463Y, D472N, R484C, A524T, P535S, N551S, A593E, I698V, V708I, V719M, S721L, and L735Q of AAV2 VP1 capsid protein as numbered prior to the insertion of the peptide, or the corresponding amino acid residue(s) of another AAV capsid protein. In some preferred embodiments, the subject variant capsid protein comprises an amino acid substitution at amino acid residue 708 of AAV2 VP1 capsid protein (SEQ ID NO:2). In particularly preferred embodiments, the subject variant capsid protein comprises a V708I amino acid substitution relative to AAV2 VP1 capsid protein (SEQ ID NO:2) and optionally further comprises one or more amino acid substitutions described herein.

In some embodiments, the subject variant AAV capsid protein confers to an rAAV increased infectivity of one or more lung cell types and also confers to the rAAV increased resistance to human AAV neutralizing antibodies compared to the resistance exhibited by AAV2 (wild type AAV serotype 2). In some cases, the rAAV exhibits increased resistance to human AAV neutralizing antibodies compared to the resistance exhibited by AAV2 (wild type AAV serotype 2). In some cases, the rAAV exhibits at least about 1.5-fold (e.g., at least about 3-fold, at least about 5-fold, at least about 10-fold, at least about 30-fold, etc.) greater resistance to human AAV neutralizing antibodies than the resistance exhibited by AAV2. In some cases, the rAAV exhibits increased transduction of one or more mammalian lung cell types in the presence of human AAV neutralizing antibodies compared to the transduction of mammalian cells exhibited by wild type AAV serotype 2 (AAV2).

In a preferred embodiment, a variant AAV capsid protein is provided comprising a) a peptide insertion in the GH-loop of the capsid protein, wherein the peptide insertion comprises, consists essentially of, or consists of an amino acid sequence selected from HDITKNI (SEQ ID NO: 12), NQDYTKT (SEQ ID NO:13), DNTVTRS (SEQ ID NO:14), SNSVQSI (SEQ ID NO: 15), NSTRHTD (SEQ ID NO:16), TNRTSPD (SEQ ID NO:17), ISDQTKH (SEQ ID NO: 18), QADTTKN (SEQ ID NO: 19), NAVKTDF (SEQ ID NO:20), TNQTLSA (SEQ ID NO:21), ENRTTSN (SEQ ID NO:22), PQQDTTH (SEQ ID NO:23), NATNHVI (SEQ ID NO:24), TNNSKPD (SEQ ID NO:25), SKLTLNN (SEQ ID NO:26), VTAGMGA (SEQ ID NO:27), PNSTTNN (SEQ ID NO:28), NSTSRID (SEQ ID NO:29), VASHTNN (SEQ ID NO:30), RSHQEIP (SEQ ID NO:31), LNTTKDI (SEQ ID NO:32), IIDATKN (SEQ ID NO:33), NHISQTN (SEQ ID NO:34), SNSAHIT (SEQ ID NO:35), STHQSNN (SEQ ID NO:36), KTPNLTS (SEQ ID NO:37), SNTPALS (SEQ ID NO:38), SPGATTN (SEQ ID NO:39), LAHDITKNIA (SEQ ID NO:40), LANQDYTKTA (SEQ ID NO: 41), LADNTVTRSA (SEQ ID NO:42), LASNSVQSIA (SEQ ID NO:43), LANSTRHTDA (SEQ ID NO:44), LATNRTSPDA (SEQ ID NO:45), LAISDQTKHA (SEQ ID NO:46), LAQADTTKNA (SEQ ID NO:47), LANAVKTDFA (SEQ ID NO:48), LATNQTLSAA (SEQ ID NO:49), LAENRTTSNA (SEQ ID NO:50), LAPQQDTTHA (SEQ ID NO:51), LANATNHVIA (SEQ ID NO:52), LATNNSKPDA (SEQ ID NO:53), LASKLTLNNA (SEQ ID NO:54), LAVTAGMGAA (SEQ ID NO:55), LAPNSTTNNA (SEQ ID NO:56), LANSTSRIDA (SEQ ID NO:57), LAVASHTNNA (SEQ ID NO:58), LARSHQEIPA (SEQ ID NO:59), LALNTTKDIA (SEQ ID NO:60), LAIIDATKNA (SEQ ID NO: 61), LANHISQTNA (SEQ ID NO:62), LASNSAHITA (SEQ ID NO:63), LASTHQSNNA (SEQ ID NO:64), LAKTPNLTSA (SEQ ID NO:65), LASNTPALSA (SEQ ID NO:66) and LASPGATTNA (SEQ ID NO:67), and b) one or more of the following amino acid substitutions compared to the amino acid sequence of AAV2 (SEQ ID NO: 2) or the corresponding substitution in another AAV parental serotype (i.e. other than AAV2), wherein the substituted amino acid(s) do not naturally occur at the corresponding positions: M1L, L15P, P34A, N57D, N66K, R81Q, Q101R, S109T, R144K, R144M, Q164K, T176P, L188I, S196Y, G226E, G236V, I2401, P250S, N312K, P363L, D368H, N449D, T456K, S463Y, D472N, R484C, A5241, P535S, N551S, A593E, I698V, V708I, V719M, S721L, L735Q and a combination thereof. In preferred embodiments, the one or more amino acid substitutions comprise a V708I substitution, Preferably, the peptide insertion site is located between amino acids 587 and 588 of AAV2 capsid or the corresponding position in the capsid protein of another AAV serotype.

In a particularly preferred embodiment, the variant AAV capsid comprises, consists essentially of, or consists of a peptide insertion comprising the amino acid sequence HDITKNI (SEQ ID NO:12) or comprising, consisting essentially of, or consisting of the amino acid sequence LAHDITKNIA (SEQ ID NO:40) between amino acids 587 and 588 of VP1 of AAV2 or the corresponding amino acids of another AAV capsid, and further comprises a V708I amino acid substitution at residue 708 relative to the amino acid sequence of AAV2 capsid (SEQ ID NO:2) or the corresponding residue of another AAV capsid. The variant AAV capsid may have at least about 85%, at least about 90%, at least about 95%, at least about 98%, or at least about 99%, or greater, amino acid sequence identity to the entire length of the amino acid sequence set forth in SEQ ID NO:2 or the corresponding parental AAV capsid. In a particularly preferred embodiment, the variant AAV capsid has an amino acid sequence having at least about 85%, at least about 90%, at least about 95%, at least about 98% sequence identity to, at least about 99% sequence identity to or is 100% identical to the following amino acid sequence (AAV102):

(SEQ ID NO: 68)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPLGLVEEPVKTAPGKKRPVE
HSPVEPDSSSGTGKAGQQPARKRLNFGQTGDADSVPDPQPLGQPPAAPS
GLGTNTMATGSGAPMADNNEGADGVGNSSGNWHCDSTWMGDRVITTSTR
TWALPTYNNHLYKQISSQSGASNDNHYFGYSTPWGYFDFNRFHCHFSPR
DWQRLINNNWGFRPKRLNFKLFNIQVKEVTQNDGTTTIANNLTSTVQVF
TDSEYQLPYVLGSAHQGCLPPFPADVFMVPQYGYLTLNNGSQAVGRSSF
YCLEYFPSQMLRTGNNFTFSYTFEDVPFHSSYAHSQSLDRLMNPLIDQY
LYYLSRTNTPSGTTTQSRLQFSQAGASDIRDQSRNWLPGPCYRQQRVSK
TSADNNNSEYSWTGATKYHLNGRDSLVNPGPAMASHKDDEEKFFPQSGV
LIFGKQGSEKTNVDIEKVMITDEEEIRTTNPVATEQYGSVSTNLQRGNL
AHDITKNIARQAATADVNTQGVLPGMVWQDRDVYLQGPIWAKIPHTDGH
FHPSPLMGGFGLKHPPPQILIKNTPVPANPSTTFSAAKFASFITQYSTG
QVSVEIEWELQKENSKRWNPEIQYTSNYNKSINVDFTVDINGVYSEPRP
IGTRYLTRNL

In another embodiment, a variant AAV capsid protein is provided comprising, consisting essentially of, or consisting of a) a peptide insertion located between amino acids 588 and 589 of VP1 of AAV1, AAV3A, AAV3B, AAV6 or AAV9, amino acids 586 and 587 of AAV4, amino acids 577 and 578 of AAV5, amino acids 589 and 590 of AAV7, or amino acids 590 to 591 of AAV8 or AAV10, the peptide insertion comprising consisting essentially of, or consisting of an amino acid sequence selected from LAHDITKNIA (SEQ ID NO: 40) and HDITKNI (SEQ ID NO:12), and b) a valine to isoleucine substitution at amino acid 709 of AAV3A or AAV3B, an alanine to isoleucine substitution at position 709 of AAV1 or AAV6, an asparagine to isoleucine substitution at amino acid 707 of AAV4 or amino acid 709 of AAV9 or a threonine to isoleucine substitution at amino acid 710 of AAV7 or amino acid 711 of AAV8 or AAV10 or a glutamine to isoleucine substitution at amino acid 697 of AAV5 and is optionally otherwise identical to any one of SEQ ID NOs: 1 and 3-12. In preferred embodiments, the variant capsid protein comprises a) a peptide insertion comprising the amino acid sequence comprising, consisting of, or consisting essentially of HDITKNI (SEQ ID NO: 12), comprising, consisting of, or consisting essentially of the amino acid sequence LAHDITKNIA (SEQ ID NO:40) between amino acids 587 and 588 of AAV2 capsid and b) a valine to isoleucine amino acid substitution at amino acid 708 compared to the amino acid sequence of AAV2, wherein the variant capsid protein comprises from 2 to 5, from 5 to 10, or from 10 to 15 amino acid substitutions.

In yet another embodiment, the variant capsid protein comprises a) a peptide insertion comprising, consisting of, or consisting essentially of the amino acid sequence HDITKNI (SEQ ID NO: 12) or comprising, consisting essentially of, or consisting of the amino acid sequence LAHDITKNIA (SEQ ID NO:40) between amino acids 587 and 588 of AAV2 capsid and is otherwise identical to the amino acid sequence of SEQ ID NO:2.

In a particularly preferred embodiment, the variant AAV capsid comprises a peptide insertion comprising consisting of, or consisting essentially of the amino acid sequence NQDYTKT (SEQ ID NO:13) or comprising, consisting essentially of, or consisting of the amino acid sequence LANQDYTKTA (SEQ ID NO:41) between amino acids 587 and 588 of VP1 of AAV2 or the corresponding amino acids of another AAV capsid, and further comprises a V708I amino acid substitution at residue 708 relative to the amino acid sequence of AAV2 capsid (SEQ ID NO:2) or the corresponding residue of another AAV capsid. The variant AAV capsid may have at least about 85%, at least about 90%, at least about 95%, at least about 98%, or at least about 99%, or greater, amino acid sequence identity to the entire length of the amino acid sequence set forth in SEQ ID NO:2 or the corresponding parental AAV capsid. In a particularly preferred embodiment, the variant AAV capsid has an amino acid sequence having at least about 85%, at least about 90%, at least about 95%, at least about 98% sequence identity to, at least about 99% sequence identity to or is 100% identical to the following amino acid sequence (AAV103):

(SEQ ID NO: 69)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPLGLVEEPVKTAPGKKRPVE
HSPVEPDSSSGTGKAGQQPARKRLNFGQTGDADSVPDPQPLGQPPAAPS
GLGTNTMATGSGAPMADNNEGADGVGNSSGNWHCDSTWMGDRVITTSTR
TWALPTYNNHLYKQISSQSGASNDNHYFGYSTPWGYFDFNRFHCHFSPR
DWQRLINNNWGFRPKRLNFKLFNIQVKEVTQNDGTTTIANNLTSTVQVF
TDSEYQLPYVLGSAHQGCLPPFPADVFMVPQYGYLTLNNGSQAVGRSSF
YCLEYFPSQMLRTGNNFTFSYTFEDVPFHSSYAHSQSLDRLMNPLIDQY
LYYLSRTNTPSGTTTQSRLQFSQAGASDIRDQSRNWLPGPCYRQQRVSK
TSADNNNSEYSWTGATKYHLNGRDSLVNPGPAMASHKDDEEKFFPQSGV
LIFGKQGSEKTNVDIEKVMITDEEEIRTTNPVATEQYGSVSTNLQRGNL
ANQDYTKTARQAATADVNTQGVLPGMVWQDRDVYLQGPIWAKIPHTDGH
FHPSPLMGGFGLKHPPPQILIKNTPVPANPSTTFSAAKFASFITQYSTG
QVSVEIEWELQKENSKRWNPEIQYTSNYNKSINVDFTVDTNGVYSEPRP
IGTRYLTRNL

In another embodiment, a variant AAV capsid protein is provided comprising a) a peptide insertion located between amino acids 588 and 589 of VP1 of AAV1, AAV3A, AAV3B, AAV6 or AAV9, amino acids 586 and 587 of AAV4, amino acids 577 and 578 of AAV5, amino acids 589 and 590 of AAV7, or amino acids 590 to 591 of AAV8 or AAV10, the peptide insertion comprising, consisting essentially of, or consisting of an amino acid sequence selected from LANQDYTKTA (SEQ ID NO:41) and NQDYTKT (SEQ ID NO:13), and b) a valine to isoleucine substitution at amino acid 709 of AAV3A or AAV3B, an alanine to isoleucine substitution at position 709 of AAV1 or AAV6, an asparagine to isoleucine substitution at amino acid 707 of AAV4 or amino acid 709 of AAV9 or a threonine to isoleucine substitution at amino acid 710 of AAV7 or amino acid 711 of AAV8 or AAV10 or a glutamine to isoleucine substitution at amino acid 697 of AAV5 and is optionally otherwise identical to any one of SEQ ID NOs: 1 and 3-12. In preferred embodiments, the variant capsid protein comprises a) a peptide insertion comprising the amino acid sequence or comprising, consisting of, or consisting essentially of NQDYTKT (SEQ ID NO:13), or comprising, consisting of, or consisting essentially of the amino acid sequence LANQDYTKTA (SEQ ID NO:41) between amino acids 587 and 588 of AAV2 capsid and b) a valine to isoleucine amino acid substitution at amino acid 708 compared to the amino acid sequence of AAV2, wherein the variant capsid protein comprises from 2 to 5, from 5 to 10, or from 10 to 15 amino acid substitutions.

In yet another embodiment, the variant capsid protein comprises a) a peptide insertion comprising the amino acid sequence NQDYTKT (SEQ ID NO:13) or comprising, consisting essentially of, or consisting of the amino acid sequence LANQDYTKTA (SEQ ID NO: 41) between amino acids 587 and 588 of AAV2 capsid and is otherwise identical to the amino acid sequence of SEQ ID NO:2.

In a particularly preferred embodiment, the variant AAV capsid comprises a peptide insertion comprising consisting of, or consisting essentially of the amino acid sequence DNTVTRS (SEQ ID NO:14) or comprising, consisting essentially of, or consisting of the amino acid sequence LADNTVTRSA (SEQ ID NO:42) between amino acids 587 and 588 of VP1 of AAV2 or the corresponding amino acids of another AAV capsid, and further comprises a V708I amino acid substitution at residue 708 relative to the amino acid sequence of AAV2 capsid (SEQ ID NO:2) or the corresponding residue of another AAV capsid. The variant AAV capsid may have at least about 85%, at least about 90%, at least about 95%, at least about 98%, or at least about 99%, or greater, amino acid sequence identity to the entire length of the amino acid sequence set forth in SEQ ID NO:2 or the corresponding parental AAV capsid. In a particularly preferred embodiment, the variant AAV capsid has an amino acid sequence having at least about 85%, at least about 90%, at least about 95%, at least about 98% sequence identity to, at least about 99% sequence identity to or is 100% identical to the following amino acid sequence (AAV104):

(SEQ ID NO: 70)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPLGLVEEPVKTAPGKKRPVE
HSPVEPDSSSGTGKAGQQPARKRLNFGQTGDADSVPDPQPLGQPPAAPS
GLGTNTMATGSGAPMADNNEGADGVGNSSGNWHCDSTWMGDRVITTSTR
TWALPTYNNHLYKQISSQSGASNDNHYFGYSTPWGYFDFNRFHCHFSPR
DWQRLINNNWGFRPKRLNFKLFNIQVKEVTQNDGTTTIANNLTSTVQVF
TDSEYQLPYVLGSAHQGCLPPFPADVFMVPQYGYLTLNNGSQAVGRSSF
YCLEYFPSQMLRTGNNFTFSYTFEDVPFHSSYAHSQSLDRLMNPLIDQY
LYYLSRTNTPSGTTTQSRLQFSQAGASDIRDQSRNWLPGPCYRQQRVSK
TSADNNNSEYSWTGATKYHLNGRDSLVNPGPAMASHKDDEEKFFPQSGV
LIFGKQGSEKTNVDIEKVMITDEEEIRTTNPVATEQYGSVSTNLQRGNL
ADNTVTRSARQAATADVNTQGVLPGMVWQDRDVYLQGPIWAKIPHTDGH
FHPSPLMGGFGLKHPPPQILIKNTPVPANPSTTFSAAKFASFITQYSTG
QVSVEIEWELQKENSKRWNPEIQYTSNYNKSINVDFTVDINGVYSEPRP
IGTRYLTRNL

In another embodiment, a variant AAV capsid protein is provided comprising a) a peptide insertion located between amino acids 588 and 589 of VP1 of AAV1, AAV3A, AAV3B, AAV6 or AAV9, amino acids 586 and 587 of AAV4, amino acids 577 and 578 of AAV5, amino acids 589 and 590 of AAV7, or amino acids 590 to 591 of AAV8 or AAV10, the peptide insertion comprising, consisting of, or consisting essentially of an amino acid sequence selected from LADNTVTRSA (SEQ ID NO:42) and DNTVTRS (SEQ ID NO:14), and b) a valine to isoleucine substitution at amino acid 709 of AAV3A or AAV3B, an alanine to isoleucine substitution at position 709 of AAV1 or AAV6, an asparagine to isoleucine substitution at amino acid 707 of AAV4 or amino acid 709 of AAV9 or a threonine to isoleucine substitution at amino acid 710 of AAV7 or amino acid 711 of AAV8 or AAV10 or a glutamine to isoleucine substitution at amino acid 697 of AAV5 and is optionally otherwise identical to any one of SEQ ID NOs: 1 and 3-12. In preferred embodiments, the variant capsid protein comprises a) a peptide insertion comprising the amino acid sequence or comprising, consisting of, consisting essentially of DNTVTRS (SEQ ID NO:14), comprising, consisting of, or consisting essentially of the amino acid sequence LADNTVTRSA (SEQ ID NO:42) between amino acids 587 and 588 of AAV2 capsid and b) a valine to isoleucine amino acid substitution at amino acid 708 compared to the amino acid sequence of AAV2, wherein the variant capsid protein comprises from 2 to 5, from 5 to 10, or from 10 to 15 amino acid substitutions.

In yet another embodiment, the variant capsid protein comprises a) a peptide insertion comprising, consisting of, or consisting essentially of the amino acid sequence DNTVTRS (SEQ ID NO: 14) or comprising, consisting essentially of, or consisting of the amino acid sequence LADNTVTRSA (SEQ ID NO:42) between amino acids 587 and 588 of AAV2 capsid and is otherwise identical to the amino acid sequence of SEQ ID NO:2.

In a particularly preferred embodiment, the variant AAV capsid comprises a peptide insertion comprising, consisting of, or consisting essentially of the amino acid sequence SNSVQSI (SEQ ID NO:15) or comprising, consisting essentially of, or consisting of the amino acid sequence LASNSVQSIA (SEQ ID NO:43) between amino acids 587 and 588 of VP1 of AAV2 or the corresponding amino acids of another AAV capsid, and further comprises a V708I amino acid substitution at residue 708 relative to the amino acid sequence of AAV2 capsid (SEQ ID NO:2) or the corresponding residue of another AAV capsid. The variant AAV capsid may have at least about 85%, at least about 90%, at least about 95%, at least about 98%, or at least about 99%, or greater, amino acid sequence identity to the entire length of the amino acid sequence set forth in SEQ ID NO:2 or the corresponding parental AAV capsid. In a particularly preferred embodiment, the variant AAV capsid has an amino acid sequence having at least about 85%, at least about 90%, at least about 95%, at least about 98% sequence identity to, at least about 99% sequence identity to or is 100% identical to the following amino acid sequence (AAV105):

(SEQ ID NO: 71)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPLGLVEEPVKTAPGKKRPVE
HSPVEPDSSSGTGKAGQQPARKRLNFGQTGDADSVPDPQPLGQPPAAPS
GLGTNTMATGSGAPMADNNEGADGVGNSSGNWHCDSTWMGDRVITTSTR
TWALPTYNNHLYKQISSQSGASNDNHYFGYSTPWGYFDFNRFHCHFSPR
DWQRLINNNWGFRPKRLNFKLFNIQVKEVTQNDGTTTIANNLTSTVQVF
TDSEYQLPYVLGSAHQGCLPPFPADVFMVPQYGYLTLNNGSQAVGRSSF
YCLEYFPSQMLRTGNNFTFSYTFEDVPFHSSYAHSQSLDRLMNPLIDQY
LYYLSRTNTPSGTTTQSRLQFSQAGASDIRDQSRNWLPGPCYRQQRVSK
TSADNNNSEYSWTGATKYHLNGRDSLVNPGPAMASHKDDEEKFFPQSGV
LIFGKQGSEKTNVDIEKVMITDEEEIRTTNPVATEQYGSVSTNLQRGNL
ASNSVQSIARQAATADVNTQGVLPGMVWQDRDVYLQGPIWAKIPHTDGH
FHPSPLMGGFGLKHPPPQILIKNTPVPANPSTTFSAAKFASFITQYSTG
QVSVEIEWELQKENSKRWNPEIQYTSNYNKSINVDFTVDINGVYSEPRP
IGTRYLTRNL

In another embodiment, a variant AAV capsid protein is provided comprising a) a peptide insertion located between amino acids 588 and 589 of VP1 of AAV1, AAV3A, AAV3B, AAV6 or AAV9, amino acids 586 and 587 of AAV4, amino acids 577 and 578 of AAV5, amino acids 589 and 590 of AAV7, or amino acids 590 to 591 of AAV8 or AAV10, the peptide insertion comprising, consisting of, or consisting essentially of an amino acid sequence selected from LASNSVQSIA (SEQ ID NO:43) and SNSVQSI (SEQ ID NO: 15), and b) a valine to isoleucine substitution at amino acid 709 of AAV3A or AAV3B, an alanine to isoleucine substitution at position 709 of AAV1 or AAV6, an asparagine to isoleucine substitution at amino acid 707 of AAV4 or amino acid 709 of AAV9 or a threonine to isoleucine substitution at amino acid 710 of AAV7 or amino acid 711 of AAV8 or AAV10 or a glutamine to isoleucine substitution at amino acid 697 of AAV5 and is optionally otherwise identical to any one of SEQ ID NOs: 1 and 3-12. In preferred embodiments, the variant capsid protein comprises a) a peptide insertion comprising the amino acid sequence or comprising, consisting of, or consisting essentially of SNSVQSI (SEQ ID NO:15), or comprising, consisting of, or consisting essentially of the amino acid sequence LASNSVQSIA (SEQ ID NO:43) between amino acids 587 and 588 of AAV2 capsid and b) a valine to isoleucine amino acid substitution at amino acid 708 compared to the amino acid sequence of AAV2, wherein the variant capsid protein comprises from 2 to 5, from 5 to 10, or from 10 to 15 amino acid substitutions.

In yet another embodiment, the variant capsid protein comprises a) a peptide insertion comprising, consisting of, or consisting essentially of the amino acid sequence SNSVQSI (SEQ ID NO:15) or comprising, consisting essentially of, or consisting of the amino acid sequence LASNSVQSIA (SEQ ID NO:43) between amino acids 587 and 588 of AAV2 capsid and is otherwise identical to the amino acid sequence of SEQ ID NO:2.

In a particularly preferred embodiment, the variant AAV capsid comprises a peptide insertion comprising, consisting of, or consisting essentially of the amino acid sequence NSTRHTD (SEQ ID NO:16) or comprising, consisting essentially of, or consisting of the amino acid sequence LANSTRHTDA (SEQ ID NO:44) between amino acids 587 and 588 of VP1 of AAV2 or the corresponding amino acids of another AAV capsid, and further comprises a V708I amino acid substitution at residue 708 relative to the amino acid sequence of AAV2 capsid (SEQ ID NO:2) or the corresponding residue of another AAV capsid. The variant AAV capsid may have at least about 85%, at least about 90%, at least about 95%, at least about 98%, or at least about 99%, or greater, amino acid sequence identity to the entire length of the amino acid sequence set forth in SEQ ID NO:2 or the corresponding parental AAV capsid. In a particularly preferred embodiment, the variant AAV capsid has an amino acid sequence having at least about 85%, at least about 90%, at least about 95%, at least about 98% sequence identity to, at least about 99% sequence identity to or is 100% identical to the following amino acid sequence (AAV106):

(SEQ ID NO: 72)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPLGLVEEPVKTAPGKKRPVE
HSPVEPDSSSGTGKAGQQPARKRLNFGQTGDADSVPDPQPLGQPPAAPS
GLGTNTMATGSGAPMADNNEGADGVGNSSGNWHCDSTWMGDRVITTSTR
TWALPTYNNHLYKQISSQSGASNDNHYFGYSTPWGYFDFNRFHCHFSPR
DWQRLINNNWGFRPKRLNFKLFNIQVKEVTQNDGTTTIANNLTSTVQVF
TDSEYQLPYVLGSAHQGCLPPFPADVFMVPQYGYLTLNNGSQAVGRSSF
YCLEYFPSQMLRTGNNFTFSYTFEDVPFHSSYAHSQSLDRLMNPLIDQY
LYYLSRTNTPSGTTTQSRLQFSQAGASDIRDQSRNWLPGPCYRQQRVSK
TSADNNNSEYSWTGATKYHLNGRDSLVNPGPAMASHKDDEEKFFPQSGV
LIFGKQGSEKTNVDIEKVMITDEEEIRTTNPVATEQYGSVSTNLQRGNL
ANSTRHTDARQAATADVNTQGVLPGMVWQDRDVYLQGPIWAKIPHTDGH
FHPSPLMGGFGLKHPPPQILIKNTPVPANPSTTFSAAKFASFITQYSTG
QVSVEIEWELQKENSKRWNPEIQYTSNYNKSINVDFTVDTNGVYSEPRP
IGTRYLTRNL

In another embodiment, a variant AAV capsid protein is provided comprising a) a peptide insertion located between amino acids 588 and 589 of VP1 of AAV1, AAV3A, AAV3B, AAV6 or AAV9, amino acids 586 and 587 of AAV4, amino acids 577 and 578 of AAV5, amino acids 589 and 590 of AAV7, or amino acids 590 to 591 of AAV8 or AAV10, the peptide insertion comprising, consisting of, or consisting essentially of an amino acid sequence selected from LANSTRHTDA (SEQ ID NO:44) and NSTRHTD (SEQ ID NO:16), and b) a valine to isoleucine substitution at amino acid 709 of AAV3A or AAV3B, an alanine to isoleucine substitution at position 709 of AAV1 or AAV6, an asparagine to isoleucine substitution at amino acid 707 of AAV4 or amino acid 709 of AAV9 or a threonine to isoleucine substitution at amino acid 710 of AAV7 or amino acid 711 of AAV8 or AAV10 or a glutamine to isoleucine substitution at amino acid 697 of AAV5 and is optionally otherwise identical to any one of SEQ ID NOs: 1 and 3-12. In preferred embodiments, the variant capsid protein comprises a) a peptide insertion comprising the amino acid sequence or comprising, consisting of or consisting essentially of NSTRHTD (SEQ ID NO: 16), or comprising, consisting of, or consisting essentially of the amino acid sequence LANSTRHTDA (SEQ ID NO:44) between amino acids 587 and 588 of AAV2 capsid and b) a valine to isoleucine amino acid substitution at amino acid 708 compared to the amino acid sequence of AAV2, wherein the variant capsid protein comprises from 2 to 5, from 5 to 10, or from 10 to 15 amino acid substitutions.

In yet another embodiment, the variant capsid protein comprises a) a peptide insertion comprising, consisting of, or consisting essentially of the amino acid sequence NSTRHTD (SEQ ID NO:16) or comprising, consisting essentially of, or consisting of the amino acid sequence LANSTRHTDA (SEQ ID NO:44) between amino acids 587 and 588 of AAV2 capsid and is otherwise identical to the amino acid sequence of SEQ ID NO:2.

In a particularly preferred embodiment, the variant AAV capsid comprises a peptide insertion comprising, consisting of, or consisting essentially of the amino acid sequence TNRTSPD (SEQ ID NO:17) or comprising, consisting essentially of, or consisting of the amino acid sequence LATNRTSPDA (SEQ ID NO:45) between amino acids 587 and 588 of VP1 of AAV2 or the corresponding amino acids of another AAV capsid, and further comprises a V708I amino acid substitution at residue 708 relative to the amino acid sequence of AAV2 capsid (SEQ ID NO:2) or the corresponding residue of another AAV capsid. The variant AAV capsid may have at least about 85%, at least about 90%, at least about 95%, at least about 98%, or at least about 99%, or greater, amino acid sequence identity to the entire length of the amino acid sequence set forth in SEQ ID NO:2 or the corresponding parental AAV capsid. In a particularly preferred embodiment, the variant AAV capsid has an amino acid sequence having at least about 85%, at least about 90%, at least about 95%, at least about 98% sequence identity to, at least about 99% sequence identity to or is 100% identical to the following amino acid sequence (AAV107):

(SEQ ID NO: 73)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPLGLVEEPVKTAPGKKRPVE
HSPVEPDSSSGTGKAGQQPARKRLNFGQTGDADSVPDPQPLGQPPAAPS
GLGTNTMATGSGAPMADNNEGADGVGNSSGNWHCDSTWMGDRVITTSTR
TWALPTYNNHLYKQISSQSGASNDNHYFGYSTPWGYFDFNRFHCHFSPR
DWQRLINNNWGFRPKRLNFKLFNIQVKEVTQNDGTTTIANNLTSTVQVF
TDSEYQLPYVLGSAHQGCLPPFPADVFMVPQYGYLTLNNGSQAVGRSSF
YCLEYFPSQMLRTGNNFTFSYTFEDVPFHSSYAHSQSLDRLMNPLIDQY
LYYLSRTNTPSGTTTQSRLQFSQAGASDIRDQSRNWLPGPCYRQQRVSK
TSADNNNSEYSWTGATKYHLNGRDSLVNPGPAMASHKDDEEKFFPQSGV
LIFGKQGSEKTNVDIEKVMITDEEEIRTTNPVATEQYGSVSTNLQRGNL
ATNRTSPDARQAATADVNTQGVLPGMVWQDRDVYLQGPIWAKIPHTDGH
FHPSPLMGGFGLKHPPPQILIKNTPVPANPSTTFSAAKFASFITQYSTG
QVSVEIEWELQKENSKRWNPEIQYTSNYNKSINVDFTVDINGVYSEPRP
IGTRYLTRNL

In another embodiment, a variant AAV capsid protein is provided comprising a) a peptide insertion located between amino acids 588 and 589 of VP1 of AAV1, AAV3A, AAV3B, AAV6 or AAV9, amino acids 586 and 587 of AAV4, amino acids 577 and 578 of AAV5, amino acids 589 and 590 of AAV7, or amino acids 590 to 591 of AAV8 or AAV10, the peptide insertion comprising, consisting of, or consisting essentially of an amino acid sequence selected from LATNRTSPDA (SEQ ID NO:45) and TNRTSPD (SEQ ID NO: 17), and b) a valine to isoleucine substitution at amino acid 709 of AAV3A or AAV3B, an alanine to isoleucine substitution at position 709 of AAV1 or AAV6, an asparagine to isoleucine substitution at amino acid 707 of AAV4 or amino acid 709 of AAV9) or a threonine to isoleucine substitution at amino acid 710 of AAV7 or amino acid 711 of AAV8 or AAV10 or a glutamine to isoleucine substitution at amino acid 697 of AAV5 and is optionally otherwise identical to any one of SEQ ID NOs: 1 and 3-12. In preferred embodiments, the variant capsid protein comprises a) a peptide insertion comprising the amino acid sequence or comprising, consisting of or consisting essentially of TNRTSPD (SEQ ID NO:17), or comprising, consisting of or consisting essentially of the amino acid sequence LATNRTSPDA (SEQ ID NO:45) between amino acids 587 and 588 of AAV2 capsid and b) a valine to isoleucine amino acid substitution at amino acid 708 compared to the amino acid sequence of AAV2, wherein the variant capsid protein comprises from 2 to 5, from 5 to 10, or from 10 to 15 amino acid substitutions.

In yet another embodiment, the variant capsid protein comprises a) a peptide insertion comprising, consisting of or consisting essentially of the amino acid sequence TNRTSPD (SEQ ID NO:17) or comprising, consisting essentially of, or consisting of the amino acid sequence LATNRTSPDA (SEQ ID NO:45) between amino acids 587 and 588 of AAV2 capsid and is otherwise identical to the amino acid sequence of SEQ ID NO:2.

In other embodiments, the variant AAV capsid comprises consists of or consists essentially of a peptide insertion comprising an amino acid sequence selected from ISDQTKH (SEQ ID NO:18), QADTTKN (SEQ ID NO:19), NAVKTDF (SEQ ID NO:20), TNQTLSA (SEQ ID NO:21), ENRTTSN (SEQ ID NO:22), PQQDTTH (SEQ ID NO:23), NATNHVI (SEQ ID NO:24), TNNSKPD (SEQ ID NO:25), SKLTLNN (SEQ ID NO:26), VTAGMGA (SEQ ID NO:27), PNSTTNN (SEQ ID NO:28), NSTSRID (SEQ ID NO:29), VASHTNN (SEQ ID NO:30), RSHQEIP (SEQ ID NO:31), LNTTKDI (SEQ ID NO:32), IIDATKN (SEQ ID NO:33), NHISQTN (SEQ ID NO:34), SNSAHIT (SEQ ID NO:35), STHQSNN (SEQ ID NO:36), KTPNLTS (SEQ ID NO:37), SNTPALS (SEQ ID NO:38), and SPGATTN (SEQ ID NO:39) or comprising, consisting essentially of, or consisting of an amino acid sequence selected from LAISDQTKHA (SEQ ID NO:46), LAQADTTKNA (SEQ ID NO:47), LANAVKTDFA (SEQ ID NO:48), LATNQTLSAA (SEQ ID NO:49), LAENRTTSNA (SEQ ID NO:50), LAPQQDTTHA (SEQ ID NO:51), LANATNHVIA (SEQ ID NO:52), LATNNSKPDA (SEQ ID NO:53), LASKLTLNNA (SEQ ID NO:54), LAVTAGMGAA (SEQ ID NO:55), LAPNSTTNNA (SEQ ID NO:56), LANSTSRIDA (SEQ ID NO:57), LAVASHTNNA (SEQ ID NO:58), LARSHQEIPA (SEQ ID NO:59), LALNTTKDIA (SEQ ID NO:60), LAIIDATKNA (SEQ ID NO:61), LANHISQTNA (SEQ ID NO: 62), LASNSAHITA (SEQ ID NO:63), LASTHQSNNA (SEQ ID NO:64), LAKTPNLTSA (SEQ ID NO:65), LASNTPALSA (SEQ ID NO:66) and LASPGATTNA (SEQ ID NO:67) between amino acids 587 and 588 of VP1 of AAV2 or the corresponding amino acids of another AAV capsid, and further comprises one or more amino acid substitutions relative to the amino acid sequence of AAV2 capsid (SEQ ID NO:2). In some aspects, the one or more amino acid substitutions comprises a V708I amino acid substitution at residue 708 relative to the amino acid sequence of AAV2 capsid (SEQ ID NO: 2) or the corresponding residue of another AAV capsid. The variant AAV capsid may have at least about 85%, at least about 90%, at least about 95%, at least about 98%, or at least about 99%, or greater, amino acid sequence identity to the entire length of the amino acid sequence set forth in SEQ ID NO:2 or the corresponding parental AAV capsid.

In related embodiments, a variant AAV capsid protein is provided comprising a) a peptide insertion located between amino acids 588 and 589 of VP1 of AAV1. AAV3A. AAV3B. AAV6 or AAV9, amino acids 586 and 587 of AAV4, amino acids 577 and 578 of AAV5, amino acids 589 and 590 of AAV7, or amino acids 590 to 591 of AAV8 or AAV10, the peptide insertion comprising, consisting of or consisting essentially of an amino acid sequence selected from HDITKNI (SEQ ID NO: 12). NQDYTKT (SEQ ID NO: 13), DNTVTRS (SEQ ID NO: 14), SNSVQSI (SEQ ID NO:15), NSTRHTD (SEQ ID NO: 16), TNRTSPD (SEQ ID NO: 17), ISDQTKH (SEQ ID NO:18), QADTTKN (SEQ ID NO: 19), NAVKTDF (SEQ ID NO:20), TNQTLSA (SEQ ID NO:21), ENRTTSN (SEQ ID NO: 22), PQQDTTH (SEQ ID NO:23), NATNHVI (SEQ ID NO:24), TNNSKPD (SEQ ID NO: 25), SKLTLNN (SEQ ID NO:26), VTAGMGA (SEQ ID NO:27), PNSTTNN (SEQ ID NO: 28), NSTSRID (SEQ ID NO:29), VASHTNN (SEQ ID NO:30), RSHQEIP (SEQ ID NO: 31), LNTTKDI (SEQ ID NO:32), IIDATKN (SEQ ID NO:33), NHISQTN (SEQ ID NO: 34), SNSAHIT (SEQ ID NO:35), STHQSNN (SEQ ID NO:36), KTPNLTS (SEQ ID NO: 37), SNTPALS (SEQ ID NO:38), SPGATTN (SEQ ID NO:39), LAHDITKNIA (SEQ ID NO: 40), LANQDYTKTA (SEQ ID NO:41), LADNTVTRSA (SEQ ID NO:42), LASNSVQSIA (SEQ ID NO:43), LANSTRHTDA (SEQ ID NO:44), LATNRTSPDA (SEQ ID NO:45), LAISDQTKHA (SEQ ID NO:46), LAQADTTKNA (SEQ ID NO:47), LANAVKTDFA (SEQ ID NO:48), LATNQTLSAA (SEQ ID NO:49), LAENRTTSNA (SEQ ID NO:50), LAPQQDTTHA (SEQ ID NO:51), LANATNHVIA (SEQ ID NO:52), LATNNSKPDA (SEQ ID NO:53), LASKLTLNNA (SEQ ID NO:54), LAVTAGMGAA (SEQ ID NO:55), LAPNSTTNNA (SEQ ID NO:56), LANSTSRIDA (SEQ ID NO:57), LAVASHTNNA (SEQ ID NO:58), LARSHQEIPA (SEQ ID NO:59), LALNTTKDIA (SEQ ID NO:60), LAIIDATKNA (SEQ ID NO:61), LANHISQTNA (SEQ ID NO:62), LASNSAHITA (SEQ ID NO:63), LASTHQSNNA (SEQ ID NO:64), LAKTPNLTSA (SEQ ID NO:65), LASNTPALSA (SEQ ID NO:66) and LASPGATTNA (SEQ ID NO:67), and b) a valine to isoleucine substitution at amino acid 709 of AAV3A or AAV3B, an alanine to isoleucine substitution at position 709 of AAV1 or AAV6, an asparagine to isoleucine substitution at amino acid 707 of AAV4 or amino acid 709 of AAV9 or a threonine to isoleucine substitution at amino acid 710 of AAV7 or amino acid 711 of AAV8 or AAV10 or a glutamine to isoleucine substitution at amino acid 697 of AAV5 and is optionally otherwise identical to any one of SEQ ID NOs: 1 and 3-12, In preferred embodiments, the variant capsid protein comprises a) a peptide insertion comprising, consisting of or consisting essentially of an amino acid sequence selected from ISDQTKH (SEQ ID NO: 18), QADTTKN (SEQ ID NO: 19), NAVKTDF (SEQ ID NO:20), TNQTLSA (SEQ ID NO:21), ENRTTSN (SEQ ID NO:22), PQQDTTH (SEQ ID NO:23), NATNHVI (SEQ ID NO:24), TNNSKPD (SEQ ID NO:25), SKLTLNN (SEQ ID NO:26), VTAGMGA (SEQ ID NO:27), PNSTTNN (SEQ ID NO:28), NSTSRID (SEQ ID NO:29), VASHTNN (SEQ ID NO:30), RSHQEIP (SEQ ID NO:31), LNTTKDI (SEQ ID NO:32), IIDATKN (SEQ ID NO:33), NHISQTN (SEQ ID NO:34), SNSAHIT (SEQ ID NO:35), STHQSNN (SEQ ID NO:36), KTPNLTS (SEQ ID NO:37), SNTPALS (SEQ ID NO:38), and SPGATTN (SEQ ID NO:39), or consisting of an amino acid sequence selected from LAISDQTKHA (SEQ ID NO:46), LAQADTTKNA (SEQ ID NO:47), LANAVKTDFA (SEQ ID NO:48), LATNQTLSAA (SEQ ID NO:49), LAENRTTSNA (SEQ ID NO:50), LAPQQDTTHA (SEQ ID NO:51), LANATNHVIA (SEQ ID NO:52), LATNNSKPDA (SEQ ID NO:53), LASKLTLNNA (SEQ ID NO:54), LAVTAGMGAA (SEQ ID NO:55), LAPNSTTNNA (SEQ ID NO:56), LANSTSRIDA (SEQ ID NO:57), LAVASHTNNA (SEQ ID NO:58), LARSHQEIPA (SEQ ID NO:59), LALNTTKDIA (SEQ ID NO:60), LAIIDATKNA (SEQ ID NO:61), LANHISQTNA (SEQ ID NO:62), LASNSAHITA (SEQ ID NO: 63), LASTHQSNNA (SEQ ID NO:64), LAKTPNLTSA (SEQ ID NO:65), LASNTPALSA (SEQ ID NO:66) and LASPGATTNA (SEQ ID NO:67) between amino acids 587 and 588 of AAV2 capsid and b) a valine to isoleucine amino acid substitution at amino acid 708 compared to the amino acid sequence of AAV2, wherein the variant capsid protein comprises from 2 to 5, from 5 to 10, or from 10 to 15 amino acid substitutions.

In yet another embodiment, the variant capsid protein comprises a) a peptide insertion comprising, consisting of or consisting essentially of an amino acid sequence selected from ISDQTKH (SEQ ID NO:18), QADTTKN (SEQ ID NO:19), NAVKTDF (SEQ ID NO:20), TNQTLSA (SEQ ID NO:21), ENRTTSN (SEQ ID NO:22), PQQDTTH (SEQ ID NO:23), NATNHVI (SEQ ID NO:24), TNNSKPD (SEQ ID NO:25), SKLTLNN (SEQ ID NO:26), VTAGMGA (SEQ ID NO:27), PNSTTNN (SEQ ID NO:28), NSTSRID (SEQ ID NO:29), VASHTNN (SEQ ID NO:30), RSHQEIP (SEQ ID NO:31), LNTTKDI (SEQ ID NO:32), IIDATKN (SEQ ID NO:33), NHISQTN (SEQ ID NO:34), SNSAHIT (SEQ ID NO:35), STHQSNN (SEQ ID NO:36), KTPNLTS (SEQ ID NO:37), SNTPALS (SEQ ID NO:38), and SPGATTN (SEQ ID NO:39), or consisting of an amino acid sequence selected from LAISDQTKHA (SEQ ID NO:46), LAQADTTKNA (SEQ ID NO: 47), LANAVKTDFA (SEQ ID NO:48), LATNQTLSAA (SEQ ID NO:49), LAENRTTSNA (SEQ ID NO:50), LAPQQDTTHA (SEQ ID NO:51), LANATNHVIA (SEQ ID NO:52), LATNNSKPDA (SEQ ID NO:53), LASKLTLNNA (SEQ ID NO:54), LAVTAGMGAA (SEQ ID NO:55), LAPNSTTNNA (SEQ ID NO:56), LANSTSRIDA (SEQ ID NO:57), LAVASHTNNA (SEQ ID NO:58), LARSHQEIPA (SEQ ID NO:59), LALNTTKDIA (SEQ ID NO:60), LAIIDATKNA (SEQ ID NO:61), LANHISQTNA (SEQ ID NO: 62), LASNSAHITA (SEQ ID NO:63), LASTHQSNNA (SEQ ID NO:64), LAKTPNLTSA (SEQ ID NO:65), LASNTPALSA (SEQ ID NO:66) and LASPGATTNA (SEQ ID NO:67) between amino acids 587 and 588 of AAV2 capsid and is otherwise identical to the amino acid sequence of SEQ ID NO:2.

In another embodiment, a variant AAV capsid protein is provided comprising a substitution of amino acids 586-597 of wt AAV2 of SEQ ID NO:2 comprising, consisting of or consisting essentially of the following amino acid sequence: VPTGaEtLNvnG (SEQ ID NO: 74); lower case letters correspond to amino acids in the wild type AAV2 sequence). Put another way, the variant AAV capsid protein comprises the following amino acid substitutions relative to AAV2: G586V. N587P. R588T. Q589. A591E. A593L. D594N and T597G. The variant AAV capsid protein may comprise the aforementioned amino acid substitutions and be otherwise identical to SEQ ID NO:2 or may be at least 80%, at least 90%, at least 95%, at least 98% or at least 99% identical to SEQ ID NO:2.

In another embodiment, a variant AAV capsid protein with improved tropism for one or more lung cells is provided comprising a substitution of amino acids 588-597 of wt AAV2 of SEQ ID NO:2 comprising, consisting of or consisting essentially of the following amino acid sequence: LAPDFTTLDA (SEQ ID NO:75). Put another way, the variant AAV capsid protein comprises the following amino acid substitutions relative to AAV2: R588L, Q589A, A590P, A591D, T592F, A593T, D594T, V595L, N596D and T597A. The variant AAV capsid protein may comprise the aforementioned amino acid substitutions and be otherwise identical to SEQ ID NO:2 or may be at least 80%, at least 90%, at least 95%, at least 98% or at least 99% identical to SEQ ID NO:2.

In another embodiment, a variant AAV capsid protein with improved tropism for one or more lung cells is provided comprising, consisting of or consisting essentially of a substitution of amino acids 575-586 of wt AAV5 of SEQ ID NO:6 (SSTTAPATGTYN; SEQ ID NO:76) with an amino acid sequence selected from TGRQNPDMSGLS (SEQ ID NO: 77) TGQRALDLRGLS (SEQ ID NO:78), TGWMSNQWLGLS (SEQ ID NO:79), TGVSQEPWAGLS (SEQ ID NO:80), TGVSLLVPSGLS (SEQ ID NO:81), TGGMGSWHSGLS (SEQ ID NO:82), TGSPLVFQAGLS (SEQ ID NO:83), TGLYDNSHVGLS (SEQ ID NO:84), TGDGDVGgGGLS (SEQ ID NO:85), TGPSpNPYtGLS (SEQ ID NO:86), TGNSGLAEAGLS (SEQ ID NO:87), and TGLYSPNDGGLS (SEQ ID NO:88). The variant AAV capsid protein may comprise the aforementioned amino acid substitutions and be otherwise identical to SEQ ID NO:6 or may be at least 80%, at least 90%, at least 95%, at least 98% or at least 99% identical to SEQ ID NO:6.

In another embodiment, a variant AAV capsid protein with improved tropism for one or more lung cells is provided comprising, consisting of or consisting essentially of a substitution of amino acids 533-545 of wt AAV5 of SEQ ID NO:6 (PANPGTTATYLEG; SEQ ID NO:89) with an amino acid sequence selected from FSPTYPSVWWFQR (SEQ ID NO: 90), VMPWgLVFVCFDF (SEQ ID NO:91), CMTAWPVDASFLN (SEQ ID NO:92), IYLRLGIYWCAGV (SEQ ID NO:93), GLGGSSIGSRTSA (SEQ ID NO:94), LFICFCCFYA (1) FF (SEQ ID NO:95), IDDDCSVaGyRSW (SEQ ID NO:96), SNGITFKDRRCLL (SEQ ID NO:97), FMIGNKVPIA (1) Pg (SEQ ID NO:98), and IYLRLGIYWCAGN (SEQ ID NO:99). In a related embodiment, a variant AAV capsid protein with improved tropism for one or more lung cells is provided comprising a substitution of amino acids 533-544 of wt AAV5 of SEQ ID NO:6 (PANPGTTATYLE; SEQ ID NO: 100) with the following amino acid sequence: LSTpFIVaGSGI (SEQ ID NO: 101). Lower case letters in each sequence correspond to amino acids in the wild type AAV5 sequence. The variant AAV capsid protein may comprise the aforementioned amino acid substitutions and be otherwise identical to SEQ ID NO:6 or may be at least 80%, at least 90%, at least 95%, at least 98% or at least 99% identical to SEQ ID NO:6.

The AAV variants disclosed herein were generated through the use of in vivo directed evolution involving the use of primate lung screens following aerosol administration. In some embodiments, the variant capsid proteins disclosed herein, when present in an AAV virion, confer increased transduction of a lung cell compared to the transduction of the lung cell by an AAV virion comprising the corresponding parental AAV capsid protein or wild-type AAV. For example, in some embodiments, the variant capsid proteins disclosed herein, when present in an AAV virion, confer more efficient transduction of primate lung cells than AAV virions comprising the corresponding parental AAV capsid protein or wild-type AAV capsid protein, e.g., the lung cells take up more AAV virions comprising the subject variant AAV capsid protein than AAV virions comprising the parental AAV capsid protein or wild-type AAV. In some such embodiments, the AAV variant virion or variant rAAV exhibits at least 2-fold, at least 5-fold, at least 10-fold, at least 15-fold, at least 20-fold, at least 25-fold, at least 50-fold, or more than 50-fold, increased transduction of a lung cell, compared to the transduction of the lung cell by a wild-type AAV virion or rAAV comprising the corresponding parental AAV capsid protein. In certain such embodiments, the variant capsid proteins disclosed herein, when present in an AAV virion, confer broader transduction of the primate lung cells than AAV virions comprising the corresponding parental AAV capsid protein or wild type AAV capsid protein. In other words, the variant AAV virion transduces cell types not transduced by virions comprising the corresponding parental AAV capsid protein, and hence more types of cells in the lung than the corresponding parental AAV virion. In some embodiments, the AAV variant virion preferentially transduces a lung cell. e.g., a subject rAAV virion infects a lung cell with 2-fold, 5-fold, 10-fold, 15-fold. 20-fold, 25-fold, 50-fold, or more than 50-fold, specificity than another lung cell or a non-lung cell, e.g., a cell outside the lung. In some embodiments, the transduced lung cell is an upper airway cell. In some embodiments, the lung cell is an upper airway epithelial cell. In some embodiments, the lung cell is an alveolar epithelium cell. In some embodiments, the lung cell is a primary, secondary or tertiary bronchial epithelial cell. In some embodiments, the lung cell is a tracheal epithelial cell. In some embodiments, the lung cell is a ciliated airway epithelial cell. In some embodiments, the lung cell is a lung alveolar epithelial type 1 (AECI) or type 2 (AECII) cell. In some embodiments, the lung cell is a smooth muscle cell. In some embodiments, the lung cell is an endothelial cell. An increase in transduction of a lung cell. e.g., increased efficiency of transduction, broader transduction, more preferential transduction, etc. may be readily assessed in vitro or in vivo by any number of methods in the art for measuring gene expression. For example, the AAV may be packaged with a genome comprising an expression cassette comprising a reporter gene. e.g., a fluorescent protein, under the control of a ubiquitous or tissue specific promoter, and the extent of transduction assessed by detecting the fluorescent protein by, e.g., fluorescence microscopy. As another example, the AAV may be packaged with a genome comprising a bar coded nucleic acid sequence, and the extent of transduction assessed by detecting the nucleic acid sequence by, e.g., PCR. As another example, the AAV may be packaged with a genome comprising an expression cassette comprising a therapeutic gene for the treatment of a lung disease, and the extent of transduction assessed by detecting the treatment of the lung disease in an afflicted patient that was administered the AAV.

Provided herein are methods of delivering a heterologous nucleic acid to a lung cell comprising contacting the lung cell with an rAAV virion comprising (i) a capsid protein comprising, consisting of or consisting essentially of a peptide insertion selected from HDITKNI (SEQ ID NO:12), NQDYTKT (SEQ ID NO:13), DNTVTRS (SEQ ID NO: 14), SNSVQSI (SEQ ID NO:15), NSTRHTD (SEQ ID NO: 16), TNRTSPD (SEQ ID NO: 17), ISDQTKH (SEQ ID NO:18), QADTTKN (SEQ ID NO: 19), NAVKTDF (SEQ ID NO: 20), TNQTLSA (SEQ ID NO:21), ENRTTSN (SEQ ID NO:22), PQQDTTH (SEQ ID NO: 23), NATNHVI (SEQ ID NO:24), TNNSKPD (SEQ ID NO:25), SKLTLNN (SEQ ID NO: 26), VTAGMGA (SEQ ID NO:27), PNSTTNN (SEQ ID NO:28), NSTSRID (SEQ ID NO: 29), VASHTNN (SEQ ID NO:30), RSHQEIP (SEQ ID NO:31), LNTTKDI (SEQ ID NO: 32), IIDATKN (SEQ ID NO:33), NHISQTN (SEQ ID NO:34), SNSAHIT (SEQ ID NO: 35), STHQSNN (SEQ ID NO:36), KTPNLTS (SEQ ID NO:37), SNTPALS (SEQ ID NO: 38), SPGATTN (SEQ ID NO:39), LAHDITKNIA (SEQ ID NO:40), LANQDYTKTA (SEQ ID NO:41), LADNTVTRSA (SEQ ID NO:42), LASNSVQSIA (SEQ ID NO:43), LANSTRHTDA (SEQ ID NO:44), LATNRTSPDA (SEQ ID NO:45), LAISDQTKHA (SEQ ID NO:46), LAQADTTKNA (SEQ ID NO:47), LANAVKTDFA (SEQ ID NO:48), LATNQTLSAA (SEQ ID NO:49), LAENRTTSNA (SEQ ID NO:50), LAPQQDTTHA (SEQ ID NO:51), LANATNHVIA (SEQ ID NO:52), LATNNSKPDA (SEQ ID NO:53), LASKLTLNNA (SEQ ID NO:54), LAVTAGMGAA (SEQ ID NO:55), LAPNSTTNNA (SEQ ID NO:56), LANSTSRIDA (SEQ ID NO:57), LAVASHTNNA (SEQ ID NO:58), LARSHQEIPA (SEQ ID NO:59), LALNTTKDIA (SEQ ID NO:60), LAIIDATKNA (SEQ ID NO: 61), LANHISQTNA (SEQ ID NO:62), LASNSAHITA (SEQ ID NO:63), LASTHQSNNA (SEQ ID NO:64), LAKTPNLTSA (SEQ ID NO:65), LASNTPALSA (SEQ ID NO:66) and LASPGATTNA (SEQ ID NO:67) and (ii) a heterologous nucleic acid comprising a nucleotide sequence encoding one or more gene products. In some embodiments, the method is an in vitro or ex vivo method. In some embodiments, the heterologous nucleic acid encodes a protein and/or short interfering RNA. In some preferred embodiments, the lung cell is any cell of the lung or trachea. In other preferred embodiments, the lung cell is an airway epithelial cell, including but not limited to an alveolar epithelium cell, a bronchial (primary, secondary or tertiary) epithelial cell or a tracheal epithelial cell. In some preferred aspects, the lung cell is a ciliated airway epithelial cell. In some preferred aspects, the lung cell is a lung alveolar epithelial type 1 (AECI) or type 2 (AECII) cell. In other embodiments, the lung cell is a smooth muscle or endothelial cell. In other embodiments, the lung cell is a basal cell, goblet cell or oocyte.

Also provided herein are methods of delivering a heterologous nucleic acid to the lung of a subject (e.g., a human subject) comprising administering to the subject an rAAV virion comprising (i) a capsid protein comprising, consisting of or consisting essentially of a peptide insertion selected from HDITKNI (SEQ ID NO:12), NQDYTKT (SEQ ID NO:13), DNTVTRS (SEQ ID NO:14), SNSVQSI (SEQ ID NO:15), NSTRHTD (SEQ ID NO:16), TNRTSPD (SEQ ID NO:17), ISDQTKH (SEQ ID NO:18), QADTTKN (SEQ ID NO:19), NAVKTDF (SEQ ID NO:20), TNQTLSA (SEQ ID NO:21), ENRTTSN (SEQ ID NO:22), PQQDTTH (SEQ ID NO:23), NATNHVI (SEQ ID NO:24), TNNSKPD (SEQ ID NO:25), SKLTLNN (SEQ ID NO:26), VTAGMGA (SEQ ID NO:27), PNSTTNN (SEQ ID NO:28), NSTSRID (SEQ ID NO:29), VASHTNN (SEQ ID NO:30), RSHQEIP (SEQ ID NO:31), LNTTKDI (SEQ ID NO:32), IIDATKN (SEQ ID NO:33), NHISQTN (SEQ ID NO:34), SNSAHIT (SEQ ID NO:35), STHQSNN (SEQ ID NO:36), KTPNLTS (SEQ ID NO:37), SNTPALS (SEQ ID NO:38), SPGATTN (SEQ ID NO:39), LAHDITKNIA (SEQ ID NO:40), LANQDYTKTA (SEQ ID NO:41), LADNTVTRSA (SEQ ID NO:42), LASNSVQSIA (SEQ ID NO:43), LANSTRHTDA (SEQ ID NO:44), LATNRTSPDA (SEQ ID NO:45), LAISDQTKHA (SEQ ID NO:46), LAQADTTKNA (SEQ ID NO:47), LANAVKTDFA (SEQ ID NO:48), LATNQTLSAA (SEQ ID NO:49), LAENRTTSNA (SEQ ID NO:50), LAPQQDTTHA (SEQ ID NO:51), LANATNHVIA (SEQ ID NO:52), LATNNSKPDA (SEQ ID NO:53), LASKLTLNNA (SEQ ID NO:54), LAVTAGMGAA (SEQ ID NO:55), LAPNSTTNNA (SEQ ID NO:56), LANSTSRIDA (SEQ ID NO:57), LAVASHTNNA (SEQ ID NO:58), LARSHQEIPA (SEQ ID NO:59), LALNTTKDIA (SEQ ID NO:60), LAIIDATKNA (SEQ ID NO:61), LANHISQTNA (SEQ ID NO: 62), LASNSAHITA (SEQ ID NO:63), LASTHQSNNA (SEQ ID NO:64), LAKTPNLTSA (SEQ ID NO:65), LASNTPALSA (SEQ ID NO:66) and LASPGATTNA (SEQ ID NO:67) and (ii) a heterologous nucleic acid comprising a nucleotide sequence encoding one or more gene products. In some embodiments, the heterologous nucleic acid encodes a protein and/or short interfering RNA. In related embodiments, methods of delivering a heterologous nucleic acid to the upper airway, nasopharynx, sinuses, mouth/buccal region and/or salivary glands of a subject (e.g., a human subject) comprising administering to the subject an rAAV virion comprising (i) a capsid protein comprising, consisting of or consisting essentially of a peptide insertion selected from HDITKNI (SEQ ID NO: 12), NQDYTKT (SEQ ID NO:13), DNTVTRS (SEQ ID NO:14), SNSVQSI (SEQ ID NO: 15), NSTRHTD (SEQ ID NO:16), TNRTSPD (SEQ ID NO:17), ISDQTKH (SEQ ID NO: 18), QADTTKN (SEQ ID NO: 19), NAVKTDF (SEQ ID NO:20), TNQTLSA (SEQ ID NO: 21), ENRTTSN (SEQ ID NO:22), PQQDTTH (SEQ ID NO:23), NATNHVI (SEQ ID NO: 24), TNNSKPD (SEQ ID NO:25), SKLTLNN (SEQ ID NO:26), VTAGMGA (SEQ ID NO:27), PNSTTNN (SEQ ID NO:28), NSTSRID (SEQ ID NO:29), VASHTNN (SEQ ID NO:30), RSHQEIP (SEQ ID NO:31), LNTTKDI (SEQ ID NO:32), IIDATKN (SEQ ID NO:33), NHISQTN (SEQ ID NO:34), SNSAHIT (SEQ ID NO:35), STHQSNN (SEQ ID NO:36), KTPNLTS (SEQ ID NO:37), SNTPALS (SEQ ID NO:38), SPGATTN (SEQ ID NO:39), LAHDITKNIA (SEQ ID NO:40), LANQDYTKTA (SEQ ID NO:41), LADNTVTRSA (SEQ ID NO:42), LASNSVQSIA (SEQ ID NO:43), LANSTRHTDA (SEQ ID NO:44), LATNRTSPDA (SEQ ID NO:45), LAISDQTKHA (SEQ ID NO:46), LAQADTTKNA (SEQ ID NO:47), LANAVKTDFA (SEQ ID NO:48), LATNQTLSAA (SEQ ID NO:49), LAENRTTSNA (SEQ ID NO:50), LAPQQDTTHA (SEQ ID NO:51), LANATNHVIA (SEQ ID NO:52), LATNNSKPDA (SEQ ID NO:53), LASKLTLNNA (SEQ ID NO:54), LAVTAGMGAA (SEQ ID NO:55), LAPNSTTNNA (SEQ ID NO:56), LANSTSRIDA (SEQ ID NO:57), LAVASHTNNA (SEQ ID NO:58), LARSHQEIPA (SEQ ID NO:59), LALNTTKDIA (SEQ ID NO:60), LAIIDATKNA (SEQ ID NO:61), LANHISQTNA (SEQ ID NO:62), LASNSAHITA (SEQ ID NO:63), LASTHQSNNA (SEQ ID NO:64), LAKTPNLTSA (SEQ ID NO:65), LASNTPALSA (SEQ ID NO:66) and LASPGATTNA (SEQ ID NO:67) and (ii) a heterologous nucleic acid comprising a nucleotide sequence encoding one or more gene products. In related aspects the rAAV or pharmaceutical composition comprising same is administered to the subject by pulmonary, endobronchial, intranasal, intratracheal, and/or intrabronchial administration. In preferred aspects, delivery of the heterologous nucleic acid to the lung of a subject delivers the one or more encoded gene products to the lung of the subject. The uses of the gene product include, but are not limited to, enhancing the level of a factor in a cell, enhancing the level of a factor in a neighboring cell through secretion of a factor, decreasing the level of a factor in a cell, or decreasing the level of a factor in a neighboring cell through secretion of a factor. The gene product can be designed to supplement the level of a defective or missing gene product (e.g., the gene product may be a therapeutic replacement gene), decrease the level of a defective gene product (e.g., the gene product may be an interfering RNA such as an siRNA or miRNA that reduces expression of the defective gene product), introduce a new supporting gene product, supplement the level of a supporting gene product, decrease the level of a hindering gene product, or both decrease the level of a hindering gene product and introduce or supplement the level of a supporting gene product.

Also provided herein are methods for treating a pulmonary disease, comprising administering to a subject in need thereof a therapeutically effective amount of a recombinant AAV (rAAV) comprising (i) a capsid protein comprising, consisting of or consisting essentially of a peptide insertion selected from HDITKNI (SEQ ID NO:12), NQDYTKT (SEQ ID NO:13), DNTVTRS (SEQ ID NO:14), SNSVQSI (SEQ ID NO:15), NSTRHTD (SEQ ID NO:16), TNRTSPD (SEQ ID NO: 17), ISDQTKH (SEQ ID NO:18), QADTTKN (SEQ ID NO:19), NAVKTDF (SEQ ID NO:20), TNQTLSA (SEQ ID NO:21), ENRTTSN (SEQ ID NO:22), PQQDTTH (SEQ ID NO:23), NATNHVI (SEQ ID NO:24), TNNSKPD (SEQ ID NO:25), SKLTLNN (SEQ ID NO:26), VTAGMGA (SEQ ID NO: 27), PNSTTNN (SEQ ID NO:28), NSTSRID (SEQ ID NO:29), VASHTNN (SEQ ID NO: 30), RSHQEIP (SEQ ID NO:31), LNTTKDI (SEQ ID NO:32), IIDATKN (SEQ ID NO: 33), NHISQTN (SEQ ID NO:34), SNSAHIT (SEQ ID NO:35), STHQSNN (SEQ ID NO: 36), KTPNLTS (SEQ ID NO:37), SNTPALS (SEQ ID NO:38), SPGATTN (SEQ ID NO: 39), LAHDITKNIA (SEQ ID NO:40), LANQDYTKTA (SEQ ID NO:41), LADNTVTRSA (SEQ ID NO:42), LASNSVQSIA (SEQ ID NO:43), LANSTRHTDA (SEQ ID NO:44), LATNRTSPDA (SEQ ID NO:45), LAISDQTKHA (SEQ ID NO:46), LAQADTTKNA (SEQ ID NO:47), LANAVKTDFA (SEQ ID NO:48), LATNQTLSAA (SEQ ID NO:49), LAENRTTSNA (SEQ ID NO:50), LAPQQDTTHA (SEQ ID NO:51), LANATNHVIA (SEQ ID NO:52), LATNNSKPDA (SEQ ID NO:53), LASKLTLNNA (SEQ ID NO:54), LAVTAGMGAA (SEQ ID NO:55), LAPNSTTNNA (SEQ ID NO:56), LANSTSRIDA (SEQ ID NO:57), LAVASHTNNA (SEQ ID NO:58), LARSHQEIPA (SEQ ID NO:59), LALNTTKDIA (SEQ ID NO:60), LAIIDATKNA (SEQ ID NO:61), LANHISQTNA (SEQ ID NO:62), LASNSAHITA (SEQ ID NO:63), LASTHQSNNA (SEQ ID NO:64), LAKTPNLTSA (SEQ ID NO:65), LASNTPALSA (SEQ ID NO:66) and LASPGATTNA (SEQ ID NO:67) and (ii) a heterologous nucleic acid comprising nucleotide sequence encoding one or more gene products, the one or more gene products operably linked to a promoter. Also provided herein is the use of a recombinant AAV (rAAV) comprising (i) a capsid protein comprising, consisting of or consisting essentially of a peptide insertion selected from HDITKNI (SEQ ID NO:12), NQDYTKT (SEQ ID NO: 13), DNTVTRS (SEQ ID NO: 14), SNSVQSI (SEQ ID NO: 15), NSTRHTD (SEQ ID NO: 16), TNRTSPD (SEQ ID NO: 17), ISDQTKH (SEQ ID NO:18), QADTTKN (SEQ ID NO: 19), NAVKTDF (SEQ ID NO:20), TNQTLSA (SEQ ID NO:21), ENRTTSN (SEQ ID NO: 22), PQQDTTH (SEQ ID NO:23), NATNHVI (SEQ ID NO:24), TNNSKPD (SEQ ID NO: 25), SKLTLNN (SEQ ID NO:26), VTAGMGA (SEQ ID NO:27), PNSTTNN (SEQ ID NO: 28), NSTSRID (SEQ ID NO:29), VASHTNN (SEQ ID NO:30), RSHQEIP (SEQ ID NO: 31), LNTTKDI (SEQ ID NO:32), IIDATKN (SEQ ID NO:33), NHISQTN (SEQ ID NO: 34), SNSAHIT (SEQ ID NO:35), STHQSNN (SEQ ID NO:36), KTPNLTS (SEQ ID NO: 37), SNTPALS (SEQ ID NO:38), SPGATTN (SEQ ID NO:39), LAHDITKNIA (SEQ ID NO: 40), LANQDYTKTA (SEQ ID NO:41), LADNTVTRSA (SEQ ID NO:42), LASNSVQSIA (SEQ ID NO:43), LANSTRHTDA (SEQ ID NO:44), LATNRTSPDA (SEQ ID NO:45), LAISDQTKHA (SEQ ID NO:46), LAQADTTKNA (SEQ ID NO:47), LANAVKTDFA (SEQ ID NO:48), LATNQTLSAA (SEQ ID NO:49), LAENRTTSNA (SEQ ID NO:50), LAPQQDTTHA (SEQ ID NO:51), LANATNHVIA (SEQ ID NO:52), LATNNSKPDA (SEQ ID NO:53), LASKLTLNNA (SEQ ID NO:54), LAVTAGMGAA (SEQ ID NO:55), LAPNSTTNNA (SEQ ID NO:56), LANSTSRIDA (SEQ ID NO:57), LAVASHTNNA (SEQ ID NO:58), LARSHQEIPA (SEQ ID NO:59), LALNTTKDIA (SEQ ID NO:60), LAIIDATKNA (SEQ ID NO:61), LANHISQTNA (SEQ ID NO:62), LASNSAHITA (SEQ ID NO:63), LASTHQSNNA (SEQ ID NO:64), LAKTPNLTSA (SEQ ID NO:65), LASNTPALSA (SEQ ID NO:66) and LASPGATTNA (SEQ ID NO:67) and (ii) a heterologous nucleic acid comprising nucleotide sequence encoding one or more gene products, the one or more gene products operably linked to a promoter, for the treatment of a pulmonary disease. Also provided herein is the use of a recombinant AAV (rAAV) comprising (i) a capsid protein comprising, consisting of or consisting essentially of a peptide insertion selected from HDITKNI (SEQ ID NO:12), NQDYTKT (SEQ ID NO: 13), DNTVTRS (SEQ ID NO:14), SNSVQSI (SEQ ID NO:15), NSTRHTD (SEQ ID NO: 16), TNRTSPD (SEQ ID NO: 17), ISDQTKH (SEQ ID NO:18), QADTTKN (SEQ ID NO: 19), NAVKTDF (SEQ ID NO:20), TNQTLSA (SEQ ID NO:21), ENRTTSN (SEQ ID NO: 22), PQQDTTH (SEQ ID NO:23), NATNHVI (SEQ ID NO:24), TNNSKPD (SEQ ID NO: 25), SKLTLNN (SEQ ID NO:26), VTAGMGA (SEQ ID NO:27), PNSTTNN (SEQ ID NO: 28), NSTSRID (SEQ ID NO:29), VASHTNN (SEQ ID NO:30), RSHQEIP (SEQ ID NO: 31), LNTTKDI (SEQ ID NO:32), IIDATKN (SEQ ID NO:33), NHISQTN (SEQ ID NO: 34), SNSAHIT (SEQ ID NO:35), STHQSNN (SEQ ID NO:36), KTPNLTS (SEQ ID NO: 37), SNTPALS (SEQ ID NO:38), SPGATTN (SEQ ID NO:39), LAHDITKNIA (SEQ ID NO: 40), LANQDYTKTA (SEQ ID NO:41), LADNTVTRSA (SEQ ID NO:42), LASNSVQSIA (SEQ ID NO:43), LANSTRHTDA (SEQ ID NO:44), LATNRTSPDA (SEQ ID NO:45), LAISDQTKHA (SEQ ID NO:46), LAQADTTKNA (SEQ ID NO:47), LANAVKTDFA (SEQ ID NO:48), LATNQTLSAA (SEQ ID NO:49), LAENRTTSNA (SEQ ID NO:50), LAPQQDTTHA (SEQ ID NO:51), LANATNHVIA (SEQ ID NO:52), LATNNSKPDA (SEQ ID NO:53), LASKLTLNNA (SEQ ID NO:54), LAVTAGMGAA (SEQ ID NO:55), LAPNSTTNNA (SEQ ID NO:56), LANSTSRIDA (SEQ ID NO:57), LAVASHTNNA (SEQ ID NO:58), LARSHQEIPA (SEQ ID NO:59), LALNTTKDIA (SEQ ID NO:60), LAIIDATKNA (SEQ ID NO:61), LANHISQTNA (SEQ ID NO:62), LASNSAHITA (SEQ ID NO:63), LASTHQSNNA (SEQ ID NO:64), LAKTPNLTSA (SEQ ID NO:65), LASNTPALSA (SEQ ID NO:66) and LASPGATTNA (SEQ ID NO:67) and (ii) a heterologous nucleic acid comprising nucleotide sequence encoding one or more gene products, the one or more gene products operably linked to a promoter, in the manufacture of a medicament for the treatment of a pulmonary disease. In some aspects, the heterologous nucleic acid comprises nucleotide sequence encoding multiple gene products, in which case expression of the multiple (e.g., 2) gene products can be mediated by multiple (e.g., 2) independent promoters or may be mediated by a single promoter, with the multiple transgenes separated by an internal ribosome entry site (IRES) or a 2A peptide sequence. In preferred embodiments, the heterologous nucleic acid encodes a therapeutic protein and/or a therapeutic short interfering RNA. In related aspects, the gene product(s) delivered by the rAAV decreases the level of a hindering gene product and/or introduces or supplements the level of a supporting gene product.

Pulmonary diseases that can be treated using a variant rAAV vector or virion and/or method disclosed herein include, but are not limited to, monogenic diseases, complex genetic diseases, acquired diseases, and traumatic injuries. In some aspects the pulmonary disease is selected from chronic obstructive pulmonary disease (COPD), idiopathic pulmonary fibrosis (IPF), cystic fibrosis, pulmonary arterial hypertension, pulmonary hypertension, lung cancer (primary, secondary and metastatic), surfactant deficiency, viral and/or bacterial infection, acute bronchitis, pneumonia (including viral, bacterial, and fungal pneumonia), respiratory tract infections (including pharyngitis, croup, aspergillus, coocidiomycosis, hantavirus pulmonary syndrome, and histoplasmosis), chemical and hypersensitivity pneumonitis, tuberculosis and other mycobacterial infections (including but not limited to Mycobacterium avium), sarcoidosis, respiratory syncytial virus, pulmonary edema, acute respiratory distress syndrome (ARDS), pneumoconiosis (including black lung disease, asbestosis, and silicosis), interstitial lung disease (including sarcoidosis and autoimmune disease), pulmonary embolism, pleural effusion, pleuritis, mesothelioma, pneumothorax, acute bronchitis, bronchiolitis (including bronchiolitis obliterans), sudden infant death syndrome, sleep apnea, bronchiectasis, bronchopulmonary dysplasia, cryptogenic organizing pneumonia, E-cigarette or vaping use associated lung injury (EVALI), Middle Eastern Respiratory Syndrome (MERS), primary ciliary dyskinesia, Severe Acute Respiratory Syndrome (SARS), alpha-1-antitrypsin deficiency, asthma, interstitial lung disease, and COVID-19 (Coronavirus Disease 2019).

In some aspects, genes that may be targeted for the treatment of IPF include, but are not limited to, SFTPA1 (surfactant A1) and Caveolin-1. Genes that may be targeted for the treatment of COPD include, but are not limited to genes encoding alpha-1-antitrypsin, alpha-1-antichymotrypsin, alpha-1-macroglobulin, matrix metalloproteinase 1 (MMP1), matrix metalloproteinase 12 (MMP12), microsomal epoxide hydrolyase, CYP1A1, Glutathione S-transferase, heme oxygenase-1, TGF-beta-1, TNF-alpha, IL-1 complex, IL-8, IL-13, human leukocyte antigen (HLA-B7 and Bw16), vitamin D binding protein, and beta-2-adrenergic receptor or biologically active portions thereof.

In some aspects, a method of treating COVID-19 is provided comprising administering to a subject in need thereof, a therapeutically effective amount of a recombinant AAV (rAAV) comprising (i) a subject variant AAV capsid protein and (ii) a heterologous nucleic acid comprising a nucleotide sequence encoding one or more gene products operably linked to one or more promoters or a pharmaceutical composition comprising the rAAV, wherein the gene product(s) knocks-down, modifies and/or overexpresses a viral gene product or host cell gene to reduce or eliminate viral pathogenicity or replication in either the lung or nasopharynx and/or expresses a neutralizing antibody against an epitope on the virus.

In some aspects, a method of treating IPF is provided comprising administering to a subject in need thereof, a therapeutically effective amount of a recombinant AAV (rAAV) comprising (i) a subject variant AAV capsid protein and (ii) a heterologous nucleic acid comprising a nucleotide sequence encoding one or more gene products operably linked to one or more promoters or a pharmaceutical composition comprising the rAAV. In some preferred embodiments, an rAAV is provided for the treatment of IPF, the rAAV comprising a subject variant AAV capsid protein and a nucleic acid comprising nucleotide sequence encoding SFTPA1 and/or Caveolin-1 or biologically active portions thereof.

In some aspects, a method of treating COPD is provided comprising administering to a subject in need thereof, a therapeutically effective amount of a recombinant AAV (rAAV) comprising (i) a subject variant AAV capsid protein and (ii) a heterologous nucleic acid comprising a nucleotide sequence encoding one or more gene products operably linked to one or more promoters or a pharmaceutical composition comprising the rAAV. In some preferred embodiments, an rAAV is provided for the treatment of COPD, the rAAV comprising a subject variant AAV capsid protein and a nucleic acid comprising nucleotide sequence encoding alpha-1-antitrypsin or a biologically active portion thereof.

In some preferred aspects, an rAAV comprising a subject variant AAV capsid and a nucleic acid encoding CFTR or a biologically active portion thereof is provided for the treatment of cystic fibrosis or a lung disease associated therewith as herein described, or for use in the manufacture of a medicament for treating cystic fibrosis or a lung disease associated therewith. Preferably the nucleotide sequence encoding CFTR or a biologically active portion thereof is operably linked to an expression control sequence. In some embodiments, the nucleotide sequence encoding human CFTR or a biologically active portion thereof encodes a native human CFTR protein and has the following sequence or a sequence at least 80%, at least 85%, at least 90%, at least 95%, at least 97%, at least 98% or at least 99% identical thereto:

(SEQ ID NO: 102)
ATGCAGAGGTCGCCTCTGGAAAAGGCCAGCGTTGTCTCCAAACTTTTTTTCAGC
TGGACCAGACCAATTTTGAGGAAAGGATACAGACAGCGCCTGGAATTGTCAGA
CATATACCAAATCCCTTCTGTTGATTCTGCTGACAATCTATCTGAAAAATTGGA
AAGAGAATGGGATAGAGAGCTGGCTTCAAAGAAAAATCCTAAACTCATTAATG
CCCTTCGGCGATGTTTTTTCTGGAGATTTATGTTCTATGGAATCTTTTTATATTT
AGGGGAAGTCACCAAAGCAGTACAGCCTCTCTTACTGGGAAGAATCATAGCTT
CCTATGACCCGGATAACAAGGAGGAACGCTCTATCGCGATTTATCTAGGCATA
GGCTTATGCCTTCTCTTTATTGTGAGGACACTGCTCCTACACCCAGCCATTTTTG
GCCTTCATCACATTGGAATGCAGATGAGAATAGCTATGTTTAGTTTGATTTATA
AGAAGACTTTAAAGCTGTCAAGCCGTGTTCTAGATAAAATAAGTATTGGACAA
CTTGTTAGTCTCCTTTCCAACAACCTGAACAAATTTGATGAAGGACTTGCATTG
GCACATTTCGTGTGGATCGCTCCTTTGCAAGTGGCACTCCTCATGGGGCTAATC
TGGGAGTTGTTACAGGCGTCTGCCTTCTGTGGACTTGGTTTCCTGATAGTCCTT
GCCCTTTTTCAGGCTGGGCTAGGGAGAATGATGATGAAGTACAGAGATCAGAG
AGCTGGGAAGATCAGTGAAAGACTTGTGATTACCTCAGAAATGATTGAAAATA
TCCAATCTGTTAAGGCATACTGCTGGGAAGAAGCAATGGAAAAAATGATTGAA
AACTTAAGACAAACAGAACTGAAACTGACTCGGAAGGCAGCCTATGTGAGATA
CTTCAATAGCTCAGCCTTCTTCTTCTCAGGGTTCTTTGTGGTGTTTTTATCTGTG
CTTCCCTATGCACTAATCAAAGGAATCATCCTCCGGAAAATATTCACCACCATC
TCATTCTGCATTGTTCTGCGCATGGCGGTCACTCGGCAATTTCCCTGGGCTGTA
CAAACATGGTATGACTCTCTTGGAGCAATAAACAAAATACAGGATTTCTTACA
AAAGCAAGAATATAAGACATTGGAATATAACTTAACGACTACAGAAGTAGTGA
TGGAGAATGTAACAGCCTTCTGGGAGGAGGGATTTGGGGAATTATTTGAGAAA
GCAAAACAAAACAATAACAATAGAAAAACTTCTAATGGTGATGACAGCCTCTT
CTTCAGTAATTTCTCACTTCTTGGTACTCCTGTCCTGAAAGATATTAATTTCAAG
ATAGAAAGAGGACAGTTGTTGGCGGTTGCTGGATCCACTGGAGCAGGCAAGAC
TTCACTTCTAATGGTGATTATGGGAGAACTGGAGCCTTCAGAGGGTAAAATTA
AGCACAGTGGAAGAATTTCATTCTGTTCTCAGTTTTCCTGGATTATGCCTGGCA
CCATTAAAGAAAATATCATCTTTGGTGTTTCCTATGATGAATATAGATACAGAA
GCGTCATCAAAGCATGCCAACTAGAAGAGGACATCTCCAAGTTTGCAGAGAAA
GACAATATAGTTCTTGGAGAAGGTGGAATCACACTGAGTGGAGGTCAACGAGC
AAGAATTTCTTTAGCAAGAGCAGTATACAAAGATGCTGATTTGTATTTATTAGA
CTCTCCTTTTGGATACCTAGATGTTTTAACAGAAAAAGAAATATTTGAAAGCTG
TGTCTGTAAACTGATGGCTAACAAAACTAGGATTTTGGTCACTTCTAAAATGGA
ACATTTAAAGAAAGCTGACAAAATATTAATTTTGCATGAAGGTAGCAGCTATT
TTTATGGGACATTTTCAGAACTCCAAAATCTACAGCCAGACTTTAGCTCAAAAC
TCATGGGATGTGATTCTTTCGACCAATTTAGTGCAGAAAGAAGAAATTCAATCC
TAACTGAGACCTTACACCGTTTCTCATTAGAAGGAGATGCTCCTGTCTCCTGGA
CAGAAACAAAAAAACAATCTTTTAAACAGACTGGAGAGTTTGGGGAAAAAAG
GAAGAATTCTATTCTCAATCCAATCAACTCTATACGAAAATTTTCCATTGTGCA
AAAGACTCCCTTACAAATGAATGGCATCGAAGAGGATTCTGATGAGCCTTTAG
AGAGAAGGCTGTCCTTAGTACCAGATTCTGAGCAGGGAGAGGCGATACTGCCT
CGCATCAGCGTGATCAGCACTGGCCCCACGCTTCAGGCACGAAGGAGGCAGTC
TGTCCTGAACCTGATGACACACTCAGTTAACCAAGGTCAGAACATTCACCGAA
AGACAACAGCATCCACACGAAAAGTGTCACTGGCCCCTCAGGCAAACTTGACT
GAACTGGATATATATTCAAGAAGGTTATCTCAAGAAACTGGCTTGGAAATAAG
TGAAGAAATTAACGAAGAAGACTTAAAGGAGTGCTTTTTTGATGATATGGAGA
GCATACCAGCAGTGACTACATGGAACACATACCTTCGATATATTACTGTCCACA
AGAGCTTAATTTTTGTGCTAATTTGGTGCTTAGTAATTTTTCTGGCAGAGGTGG
CTGCTTCTTTGGTTGTGCTGTGGCTCCTTGGAAACACTCCTCTTCAAGACAAAG
GGAATAGTACTCATAGTAGAAATAACAGCTATGCAGTGATTATCACCAGCACC
AGTTCGTATTATGTGTTTTACATTTACGTGGGAGTAGCCGACACTTTGCTTGCT
ATGGGATTCTTCAGAGGTCTACCACTGGTGCATACTCTAATCACAGTGTCGAAA
ATTTTACACCACAAAATGTTACATTCTGTTCTTCAAGCACCTATGTCAACCCTC
AACACGTTGAAAGCAGGTGGGATTCTTAATAGATTCTCCAAAGATATAGCAAT
TTTGGATGACCTTCTGCCTCTTACCATATTTGACTTCATCCAGTTGTTATTAATT
GTGATTGGAGCTATAGCAGTTGTCGCAGTTTTACAACCCTACATCTTTGTTGCA
ACAGTGCCAGTGATAGTGGCTTTTATTATGTTGAGAGCATATTTCCTCCAAACC
TCACAGCAACTCAAACAACTGGAATCTGAAGGCAGGAGTCCAATTTTCACTCA
TCTTGTTACAAGCTTAAAAGGACTATGGACACTTCGTGCCTTCGGACGGCAGCC
TTACTTTGAAACTCTGTTCCACAAAGCTCTGAATTTACATACTGCCAACTGGTT
CTTGTACCTGTCAACACTGCGCTGGTTCCAAATGAGAATAGAAATGATTTTTGT
CATCTTCTTCATTGCTGTTACCTTCATTTCCATTTTAACAACAGGAGAAGGAGA
AGGAAGAGTTGGTATTATCCTGACTTTAGCCATGAATATCATGAGTACATTGCA
GTGGGCTGTAAACTCCAGCATAGATGTGGATAGCTTGATGCGATCTGTGAGCC
GAGTCTTTAAGTTCATTGACATGCCAACAGAAGGTAAACCTACCAAGTCAACC
AAACCATACAAGAATGGCCAACTCTCGAAAGTTATGATTATTGAGAATTCACA
CGTGAAGAAAGATGACATCTGGCCCTCAGGGGGCCAAATGACTGTCAAAGATC
TCACAGCAAAATACACAGAAGGTGGAAATGCCATATTAGAGAACATTTCCTTC
TCAATAAGTCCTGGCCAGAGGGTGGGCCTCTTGGGAAGAACTGGATCAGGGAA
GAGTACTTTGTTATCAGCTTTTTTGAGACTACTGAACACTGAAGGAGAAATCCA
GATCGATGGTGTGTCTTGGGATTCAATAACTTTGCAACAGTGGAGGAAAGCCT
TTGGAGTGATACCACAGAAAGTATTTATTTTTTCTGGAACATTTAGAAAAAACT
TGGATCCCTATGAACAGTGGAGTGATCAAGAAATATGGAAAGTTGCAGATGAG
GTTGGGCTCAGATCTGTGATAGAACAGTTTCCTGGGAAGCTTGACTTTGTCCTT
GTGGATGGGGGCTGTGTCCTAAGCCATGGCCACAAGCAGTTGATGTGCTTGGC
TAGATCTGTTCTCAGTAAGGCGAAGATCTTGCTGCTTGATGAACCCAGTGCTCA
TTTGGATCCAGTAACATACCAAATAATTAGAAGAACTCTAAAACAAGCATTTG
CTGATTGCACAGTAATTCTCTGTGAACACAGGATAGAAGCAATGCTGGAATGC
CAACAATTTTTGGTCATAGAAGAGAACAAAGTGCGGCAGTACGATTCCATCCA
GAAACTGCTGAACGAGAGGAGCCTCTTCCGGCAAGCCATCAGCCCCTCCGACA
GGGTGAAGCTCTTTCCCCACCGGAACTCAAGCAAGTGCAAGTCTAAGCCCCAG
ATTGCTGCTCTGAAAGAGGAGACAGAAGAAGAGGTGCAAGATACAAGGCTTT
AG

In preferred embodiments, the nucleotide sequence encoding human CFTR or a biologically activated truncated CFTR protein is codon optimized for expression in humans. In some embodiments, the nucleotide sequence encodes a biologically active truncated human CFTR protein lacking amino acids 708-759 and comprises the following nucleotide sequence or a sequence at least 80%, at least 85%, at least 90%, at least 95%, at least 97%, at least 98% or at least 99% identical thereto:

(SEQ ID NO: 103)
ATGCAGCGCAGCCCACTGGAGAAGGCAAGCGTGGTGTCCAAGCTGTTCTTTTC
CTGGACCAGGCCTATCCTGAGGAAGGGATACAGGCAGCGGCTGGAGCTGAGC
GACATCTATCAGATCCCTTCTGTGGACAGCGCCGATAATCTGTCCGAGAAGCTG
GAGAGAGAGTGGGATAGGGAGCTGGCCTCTAAGAAGAACCCAAAGCTGATCA
ATGCCCTGCGGAGATGCTTCTTTTGGCGGTTCATGTTCTACGGCATCTTCCTGTA
TCTGGGCGAGGTGACCAAGGCCGTGCAGCCACTGCTGCTGGGCAGAATCATCG
CCTCTTACGACCCCGATAACAAGGAGGAGAGGAGCATCGCCATCTATCTGGGC
ATCGGCCTGTGCCTGCTGTTTATCGTGAGGACACTGCTGCTGCACCCAGCCATC
TTCGGCCTGCACCACATCGGCATGCAGATGAGAATCGCCATGTTCAGCCTGATC
TACAAGAAGACCCTGAAGCTGAGCTCCAGGGTGCTGGACAAGATCTCCATCGG
CCAGCTGGTGTCCCTGCTGTCTAACAATCTGAACAAGTTTGATGAGGGACTGGC
CCTGGCACACTTCGTGTGGATCGCACCACTGCAGGTGGCCCTGCTGATGGGCCT
GATCTGGGAGCTGCTGCAGGCAAGCGCCTTTTGCGGACTGGGCTTCCTGATCGT
GCTGGCCCTGTTCCAGGCAGGACTGGGACGCATGATGATGAAGTACAGAGACC
AGAGGGCCGGCAAGATCTCTGAGCGGCTGGTCATCACCAGCGAGATGATCGAG
AACATCCAGTCCGTGAAGGCCTATTGTTGGGAGGAGGCCATGGAGAAGATGAT
CGAGAATCTGCGCCAGACAGAGCTGAAGCTGACCAGAAAGGCCGCCTACGTG
AGGTACTTCAACTCTAGCGCCTTCTTTTTCTCTGGCTTTTTCGTGGTGTTCCTGA
GCGTGCTGCCATACGCCCTGATCAAGGGCATCATCCTGCGGAAGATCTTTACCA
CAATCTCCTTCTGCATCGTGCTGAGAATGGCCGTGACAAGGCAGTTTCCCTGGG
CCGTGCAGACCTGGTATGACTCTCTGGGCGCCATCAATAAGATCCAGGATTTCC
TGCAGAAGCAGGAGTACAAGACACTGGAGTATAACCTGACCACAACCGAGGT
GGTCATGGAGAATGTGACCGCCTTCTGGGAGGAGGGCTTTGGCGAGCTGTTCG
AGAAGGCCAAGCAGAACAATAACAATCGCAAGACATCTAACGGCGACGATAG
CCTGTTTTTCAGCAATTTTTCCCTGCTGGGCACCCCCGTGCTGAAGGACATCAA
CTTCAAGATCGAGAGGGGACAGCTGCTGGCAGTGGCAGGCTCCACAGGCGCCG
GCAAGACCTCTCTGCTGATGATGATCATGGGCGAGCTGGAGCCAAGCGAGGGC
AAGATCAAGCACTCCGGCCGGATCTCTTTTTGCAGCCAGTTCTCCTGGATCATG
CCCGGCACCATCAAGGAGAATATCATCTTTGGCGTGTCCTACGATGAGTACAG
ATATAGGTCTGTGATCAAGGCCTGTCAGCTGGAGGAGGACATCAGCAAGTTCG
CCGAGAAGGATAACATCGTGCTGGGCGAGGGCGGCATCACACTGAGCGGAGG
ACAGAGGGCAAGGATCTCCCTGGCCAGAGCCGTGTACAAGGACGCCGATCTGT
ATCTGCTGGACAGCCCCTTTGGCTATCTGGATGTGCTGACCGAGAAGGAGATCT
TCGAGTCCTGCGTGTGCAAGCTGATGGCCAATAAGACAAGGATCCTGGTGACC
TCTAAGATGGAGCACCTGAAGAAGGCCGACAAGATCCTGATCCTGCACGAGGG
CTCCTCTTACTTTTATGGCACATTCAGCGAGCTGCAGAATCTGCAGCCTGACTT
CAGCTCCAAGCTGATGGGCTGTGACTCCTTTGATCAGTTCTCTGCCGAGAGGCG
CAACTCCATCCTGACAGAGACCCTGCACAGATTCTCTCTGGAGGGCGACGCAC
CCGTGAGCTGGACAGAGACCAAGAAGCAGTCCTTTAAGCAGACCGGCGAGTTC
GGCGAGAAGAGGAAGAATTCTATCCTGAACCCTATCAATAGCACACTGCAGGC
CCGGAGAAGGCAGTCTGTGCTGAACCTGATGACCCACAGCGTGAACCAGGGCC
AGAATATCCACAGAAAGACAACCGCCAGCACAAGGAAGGTGTCCCTGGCACCT
CAGGCAAACCTGACCGAGCTGGACATCTACTCCCGCCGGCTGTCTCAGGAGAC
CGGACTGGAGATCTCTGAGGAGATCAATGAGGAGGATCTGAAGGAGTGCTTTT
TCGACGATATGGAGAGCATCCCAGCCGTGACAACCTGGAACACATACCTGCGC
TATATCACCGTGCACAAGTCCCTGATCTTTGTGCTGATCTGGTGTCTGGTCATCT
TCCTGGCAGAGGTGGCAGCATCTCTGGTGGTGCTGTGGCTGCTGGGCAACACA
CCCCTGCAGGACAAGGGCAATTCTACCCACAGCCGCAACAATTCCTACGCCGT
GATCATCACATCTACCTCTAGCTACTACGTGTTCTACATCTATGTGGGCGTGGC
CGATACACTGCTGGCCATGGGCTTTTTCCGGGGCCTGCCCCTGGTGCACACACT
GATCACCGTGAGCAAGATCCTGCACCACAAGATGCTGCACAGCGTGCTGCAGG
CCCCTATGTCCACACTGAACACCCTGAAGGCCGGCGGCATCCTGAATCGGTTTT
CCAAGGACATCGCCATCCTGGACGATCTGCTGCCTCTGACCATCTTTGATTTCA
TCCAGCTGCTGCTGATCGTGATCGGAGCAATCGCAGTGGTGGCCGTGCTGCAG
CCTTACATCTTCGTGGCCACAGTGCCAGTGATCGTGGCCTTTATCATGCTGCGC
GCCTATTTCCTGCAGACCAGCCAGCAGCTGAAGCAGCTGGAGAGCGAGGGCCG
GTCCCCTATCTTTACACACCTGGTGACCTCCCTGAAGGGACTGTGGACACTGAG
GGCCTTCGGCCGGCAGCCATACTTTGAGACCCTGTTCCACAAGGCCCTGAACCT
GCACACAGCCAATTGGTTTCTGTATCTGAGCACCCTGCGCTGGTTTCAGATGCG
GATCGAGATGATCTTCGTGATCTTTTTCATCGCCGTGACCTTCATCTCCATCCTG
ACAACCGGAGAGGGAGAGGGAAGAGTGGGAATCATCCTGACACTGGCCATGA
ACATCATGTCTACCCTGCAGTGGGCCGTGAATTCCTCTATCGACGTGGATAGCC
TGATGAGATCTGTGAGCAGGGTGTTTAAGTTCATCGACATGCCCACAGAGGGC
AAGCCTACAAAGAGCACCAAGCCATACAAGAACGGCCAGCTGTCCAAAGTGA
TGATCATCGAGAATTCTCACGTGAAGAAGGACGATATCTGGCCATCCGG

Preferably the nucleotide sequence encoding CFTR or a biologically active portion thereof is operably linked to an expression control sequence. In some aspects, the promoter is a constitutive promoter, optionally a truncated cytomegalovirus immediate/early (CMVie) enhancer/promoter and is operably linked to the nucleotide sequence encoding the human CFTR or biologically active portion thereof. In other aspects, the promoter is a tissue specific promoter, preferably wherein the promoter directs preferential expression of the nucleic acid in a lung cell, and is operably linked to the nucleotide sequence encoding the human CFTR or biologically active portion thereof. In preferred embodiments, the promoter is a truncated CMVie promoter and is operably linked to the nucleotide sequence encoding human CFTR or a biologically active portion thereof. In a particularly preferred embodiment, the CMVie promoter is CMV173 having the following sequence or a sequence at least 90%, at least 95%, at least 98% or at least 99% identical thereto:

(SEQ ID NO: 104)
ACTCACGGGGATTTCCAAGTCTCCACCCCATTGACGTCAATGGGAGTTT
GTTTTGGCACCAAAATCAACGGGACTTTCCAAAATGTCGTAATAACCCC
GCCCCGTTGACGCAAATGGGCGGTAGGCGTGTACGGTGGGAGGTCTATA
TAAGCAGAGCTCGTTTAGTGAACCGT

In a particularly preferred embodiment, the rAAV vector comprises (i) a capsid protein comprising, consisting of or consisting essentially of a peptide insertion selected from HDITKNI (SEQ ID NO:12), NQDYTKT (SEQ ID NO: 13), DNTVTRS (SEQ ID NO: 14), SNSVQSI (SEQ ID NO:15), NSTRHTD (SEQ ID NO: 16), TNRTSPD (SEQ ID NO: 17), ISDQTKH (SEQ ID NO:18), QADTTKN (SEQ ID NO: 19), NAVKTDF (SEQ ID NO: 20), TNQTLSA (SEQ ID NO:21), ENRTTSN (SEQ ID NO:22). PQQDTTH (SEQ ID NO: 23), NATNHVI (SEQ ID NO:24), TNNSKPD (SEQ ID NO:25), SKLTLNN (SEQ ID NO: 26), VTAGMGA (SEQ ID NO:27). PNSTTNN (SEQ ID NO:28), NSTSRID (SEQ ID NO: 29), VASHTNN (SEQ ID NO:30), RSHQEIP (SEQ ID NO:31), LNTTKDI (SEQ ID NO: 32), IIDATKN (SEQ ID NO:33), NHISQTN (SEQ ID NO:34), SNSAHIT (SEQ ID NO: 35), STHQSNN (SEQ ID NO:36), KTPNLTS (SEQ ID NO:37), SNTPALS (SEQ ID NO: 38), SPGATTN (SEQ ID NO:39), LAHDITKNIA (SEQ ID NO:40), LANQDYTKTA (SEQ ID NO:41), LADNTVTRSA (SEQ ID NO:42). LASNSVQSIA (SEQ ID NO:43), LANSTRHTDA (SEQ ID NO:44), LATNRTSPDA (SEQ ID NO:45). LAISDQTKHA (SEQ ID NO:46), LAQADTTKNA (SEQ ID NO:47), LANAVKTDFA (SEQ ID NO:48), LATNQTLSAA (SEQ ID NO:49), LAENRTTSNA (SEQ ID NO:50), LAPQQDTTHA (SEQ ID NO:51), LANATNHVIA (SEQ ID NO:52), LATNNSKPDA (SEQ ID NO:53), LASKLTLNNA (SEQ ID NO:54), LAVTAGMGAA (SEQ ID NO:55), LAPNSTTNNA (SEQ ID NO:56), LANSTSRIDA (SEQ ID NO:57), LAVASHTNNA (SEQ ID NO:58). LARSHQEIPA (SEQ ID NO:59), LALNTTKDIA (SEQ ID NO:60), LAIIDATKNA (SEQ ID NO: 61). LANHISQTNA (SEQ ID NO:62). LASNSAHITA (SEQ ID NO:63). LASTHQSNNA (SEQ ID NO:64), LAKTPNLTSA (SEQ ID NO:65), LASNTPALSA (SEQ ID NO:66) and LASPGATTNA (SEQ ID NO:67) and (ii) a nucleic acid comprising from 5′ to 3′: (a) an AAV2 terminal repeat (b) a promoter (c) a nucleotide sequence encoding a human cystic fibrosis transmembrane conductance regulator (CFTR) protein or a biologically active truncated CFTR protein lacking amino acids 708-759 of the human CFTR protein sequence (d) a polyadenylation sequence and (e) an AAV2 terminal repeat.

In some aspects, the subject is administered an amount of the rAAV effective to ameliorate one or more characteristics of cystic fibrosis, nonlimiting examples of which include upper and lower airway inflammation, aberrant epithelia cytokine signaling and elevated IgE levels. In other aspects, provided herein are methods for treating lung disease associated with cystic fibrosis, including but not limited to upper airway disease, lower airway disease, nasopharyngeal disease, sinusitis and/or salivary disease associated with cystic fibrosis, comprising administering to the subject a therapeutically effective amount of an infectious rAAV comprising (i) a capsid protein comprising, consisting of or consisting essentially of a peptide insertion selected from HDITKNI (SEQ ID NO:12). NQDYTKT (SEQ ID NO:13), DNTVTRS (SEQ ID NO:14), SNSVQSI (SEQ ID NO:15), NSTRHTD (SEQ ID NO:16), TNRTSPD (SEQ ID NO: 17), ISDQTKH (SEQ ID NO:18), QADTTKN (SEQ ID NO:19), NAVKTDF (SEQ ID NO:20), TNQTLSA (SEQ ID NO:21), ENRTTSN (SEQ ID NO:22), PQQDTTH (SEQ ID NO:23), NATNHVI (SEQ ID NO:24), TNNSKPD (SEQ ID NO:25), SKLTLNN (SEQ ID NO:26), VTAGMGA (SEQ ID NO: 27), PNSTTNN (SEQ ID NO:28), NSTSRID (SEQ ID NO:29), VASHTNN (SEQ ID NO: 30), RSHQEIP (SEQ ID NO:31), LNTTKDI (SEQ ID NO:32), IIDATKN (SEQ ID NO: 33), NHISQTN (SEQ ID NO:34), SNSAHIT (SEQ ID NO:35), STHQSNN (SEQ ID NO: 36), KTPNLTS (SEQ ID NO:37), SNTPALS (SEQ ID NO:38), SPGATTN (SEQ ID NO: 39), LAHDITKNIA (SEQ ID NO:40), LANQDYTKTA (SEQ ID NO:41), LADNTVTRSA (SEQ ID NO:42), LASNSVQSIA (SEQ ID NO:43), LANSTRHTDA (SEQ ID NO:44), LATNRTSPDA (SEQ ID NO:45), LAISDQTKHA (SEQ ID NO:46), LAQADTTKNA (SEQ ID NO:47), LANAVKTDFA (SEQ ID NO:48), LATNQTLSAA (SEQ ID NO:49), LAENRTTSNA (SEQ ID NO:50), LAPQQDTTHA (SEQ ID NO:51), LANATNHVIA (SEQ ID NO:52), LATNNSKPDA (SEQ ID NO:53), LASKLTLNNA (SEQ ID NO:54), LAVTAGMGAA (SEQ ID NO:55), LAPNSTTNNA (SEQ ID NO:56), LANSTSRIDA (SEQ ID NO:57), LAVASHTNNA (SEQ ID NO:58), LARSHQEIPA (SEQ ID NO:59), LALNTTKDIA (SEQ ID NO:60), LAIIDATKNA (SEQ ID NO:61), LANHISQTNA (SEQ ID NO:62), LASNSAHITA (SEQ ID NO:63), LASTHQSNNA (SEQ ID NO:64), LAKTPNLTSA (SEQ ID NO:65), LASNTPALSA (SEQ ID NO:66) and LASPGATTNA (SEQ ID NO:67) and (ii) a nucleic acid comprising a nucleotide sequence encoding a human cystic fibrosis transmembrane conductance regulator (CFTR) protein or a biologically active truncated CFTR protein lacking amino acids 708-759 of the human CFTR protein sequence operably linked to a promoter.

rAAV gene therapy vectors of the present invention comprising a subject variant capsid protein may be administered to a patient by a variety of means to achieve and maintain a therapeutically effective level of gene product (e.g., CFTR or a biologically active portion thereof) in the target cell (e.g., upper airway cell). In some aspects, the infectious rAAV is administered to a subject (e.g., a subject with cystic fibrosis) in one or more dosages, each dosage comprising between about 1×10¹³ to about 1×10¹⁵ vector genomes (vg), about 1×10¹³ to about 1×10¹⁴ vg, between about 1×10¹⁴ and about 1×10¹⁵ vg, or between about 1×10¹⁵ and about 5×10¹⁵ vg. In some preferred aspects, each dosage comprises about 1×10¹⁴ vg or about 1×10¹⁵ vg of the rAAV. In other aspects, at least one dose of about 10¹² to 10¹⁴ vector genomes (vg)/kg of the rAAV is administered to a subject to treat a pulmonary disease. In related aspects, the subject is administered about 1×10¹¹ to about 1×10¹⁴ vg/kg, about 1×10¹² to about 9×10¹³ vg/kg, about 1×10¹² vg/kg to about 9×10¹² vg/kg, preferably about 2×10¹² vg/kg to about 3×10¹² vg/kg, more preferably about 2.6×10¹² vg/kg, about 2.7×10¹² vg/kg, about 2.8×10¹² vg/kg, about 2.9×10¹² vg/kg about 3.0×10¹² vg/kg or about 3.1×10¹² vg/kg.

In some aspects, the treatment comprises no more than a single dose administration to the subject and is effective to achieve a durable and maintained therapeutic concentration of gene product (e.g., CFTR or biologically active portion thereof). In related aspects, the treatment comprises no more than a single dose administration by inhalation of about 1×10¹³ to about 1×10¹⁵ plaque forming units (pfu), virus particles (vp) or virus genomes (vg) of rAAV comprising a subject variant capsid protein and a heterologous nucleic acid encoding a gene product to a human (e.g., a human with cystic fibrosis). In other aspects, the dosage treatment may be a multiple dose schedule. Methods pertaining to the administration of AAV vectors to humans have been previously described by Kay et al. (2000, Nat Genet 24:257-261), the entire content of which is incorporated herein by reference. In some preferred embodiments, the infectious rAAV is administered to the subject by pulmonary, endobronchial, intranasal, intratracheal, and/or intrabronchial administration. In some preferred embodiments, the infectious rAAV is administered by inhalation of an aerosol suspension comprising the rAAV (e.g. via a nebulizer).

For the purposes of the invention, the disclosure herein provides an isolated nucleic acid comprising a nucleotide sequence that encodes a subject variant AAV capsid protein as described above. An isolated nucleic acid can be an AAV vector, e.g., a recombinant AAV vector.

The disclosure herein further provides host cells such as, without limitation, isolated (genetically modified) host cells comprising a subject nucleic acid. A host cell according to the invention disclosed herein, can be an isolated cell, such as a cell from an in vitro cell culture. Such a host cell is useful for producing a subject rAAV variant virion, as described herein. In one embodiment, such a host cell is stably genetically modified with a nucleic acid. In other embodiments, a host cell is transiently genetically modified with a nucleic acid. Such a nucleic acid is introduced stably or transiently into a host cell, using established techniques, including, but not limited to, electroporation, calcium phosphate precipitation, liposome-mediated transfection, and the like. For stable transformation, a nucleic acid will generally further include a selectable marker, e.g., any of several well-known selectable markers such as neomycin resistance, and the like. Such a host cell is generated by introducing a nucleic acid into any of a variety of cells, e.g., mammalian cells, including, e.g., murine cells, and primate cells (e.g., human cells). Exemplary mammalian cells include, but are not limited to, primary cells and cell lines, where exemplary cell lines include, but are not limited to, 293 cells, COS cells, HeLa cells, Vero cells, 3T3 mouse fibroblasts, C3H10T1/2 fibroblasts, CHO cells, and the like, Exemplary host cells include, without limitation, HeLa cells (e.g., American Type Culture Collection (ATCC) No. CCL-2), CHO cells (e.g., ATCC Nos. CRL9618, CCL61, CRL9096), 293 cells (e.g., ATCC No, CRL-1573), Vero cells, NIH 3T3 cells (e.g., ATCC No. CRL-1658), Huh-7 cells, BHK cells (e.g., ATCC No. CCL10), PC12 cells (ATCC No. CRL1721). COS cells. COS-7 cells (ATCC No. CRL1651), RATI cells, mouse L cells (ATCC No. CCL1.3), human embryonic kidney (HEK) cells (ATCC No, CRL1573), HLHepG2 cells, and the like. A host cell can also be made using a baculovirus to infect insect cells such as Sf) cells, which produce AAV (see, e.g., U.S. Pat. No. 7,271,002: U.S. patent application Ser. No. 12/297,958). In some embodiments, a genetically modified host cell includes, in addition to a nucleic acid comprising a nucleotide sequence encoding a variant AAV capsid protein, as described above, a nucleic acid that comprises a nucleotide sequence encoding one or more AAV rep proteins. In other embodiments, a host cell further comprises an rAAV variant vector. An rAAV variant virion can be generated using such host cells. Methods of generating an rAAV virion are described in, e.g., U.S. Patent Publication No. 2005/0053922 and U.S. Patent Publication No, 2009/0202490.

In some embodiments of the variant rAAV vector disclosed herein, a nucleotide sequence encoding a gene product of interest is operably linked to a constitutive promoter. Suitable constitutive promoters include e.g., cytomegalovirus promoter (CMV) (Stinski et al. (1985) Journal of Virology 55 (2): 431-441), CMV early enhancer/chicken p-actin (CBA) promoter/rabbit β-globin intron (CAG) (Miyazaki et al. (1989) Gene 79 (2): 269-277, C^{SB} (Jacobson et al. (2006) Molecular Therapy 13 (6): 1074-1084), human elongation factor 1α promoter (EF1α) (Kim et al. (1990) Gene 91 (2): 217-223), human phosphoglycerate kinase promoter (PGK) (Singer-Sam et al. (1984) Gene 32 (3): 409-417, mitochondrial heavy-strand promoter (Loderio et al. (2012) PNAS 109 (17): 6513-6518), ubiquitin promoter (Wulff et al. (1990) FEBS Letters 261:101-105). In other embodiments, a nucleotide sequence encoding a gene product of interest is operably linked to an inducible promoter. In some instances, a nucleotide sequence encoding a gene product of interest is operably linked to a tissue-specific or cell type-specific regulatory element. For example, in some instances, a nucleotide sequence encoding a gene product of interest is operably linked to a lung-specific regulatory element, e.g., a regulatory element that confers selective expression of the operably linked gene in a lung cell. Lung specific promoters include, without limitation, surfactant protein B (SPB) gene promoter and the surfactant protein C (SPC) promoter.

Also provided herein are pharmaceutical compositions comprising: a) an rAAV comprising a subject variant AAV capsid protein and a heterologous nucleic acid encoding one or more gene products; and b) a pharmaceutically acceptable carrier, diluent, excipient, or buffer. In some preferred embodiments, the nucleic acid comprises a nucleotide sequence encoding a therapeutic gene and/or encoding an interfering RNA. In some embodiments, the pharmaceutically acceptable carrier, diluent, excipient, or buffer is suitable for use in a human or non-human patient. Such excipients, carriers, diluents, and buffers include any pharmaceutical agent that can be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, surfactants, pH buffering substances, and the like, may be present in such vehicles. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins: Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

In some embodiments, the pharmaceutical composition comprises 1×10⁸ to 1×10¹⁵ vector particles or vector genomes, 1×10¹⁰ to 1×10¹³ vector particles or vector genomes, or about 1×10¹⁰, about 2×10¹⁰, 3×10¹⁰, about 4×10¹⁰, about 5×10¹⁰, about 6×10¹⁰, about 7×10¹⁰, about 8×10¹⁰, about 9×10¹⁰, about 1×10¹¹, about 2×10¹¹, about 3×10¹¹, about 4×10¹¹, about 5×10¹¹, about 6×10¹¹, about 7×10¹¹, about 8×10¹¹, about 9×10¹¹, about 1×10¹², about 2×10¹², about 3×10¹², about 4×10¹², about 5×10¹², about 6×10¹², about 7×10¹², about 8×10¹², about 9×10¹² or about 1×10¹³ vector particles or vector genomes. In some aspects, the pharmaceutical composition comprises about 1×10¹¹ to about 1×10¹² vector particles or vector genomes.

### EXAMPLES

The following examples illustrate preferred embodiments of the present invention and are not intended to limit the scope of the invention in any way. While this invention has been described in relation to its preferred embodiments, various modifications thereof will be apparent to one skilled in the art from reading this application.

### Example 1

A directed evolution screen was employed to identify AAV capsid variants capable of conferring more efficient transduction of the primate lung and enhanced gene delivery efficiency to primate lung upper airway cells following intratracheal aerosol administration to non-human primates (NHP). The selection process incorporated the use of delivery to NHP lungs in vivo and human lung cultures in vitro.

### Methods

### Therapeutic Vector Evolution

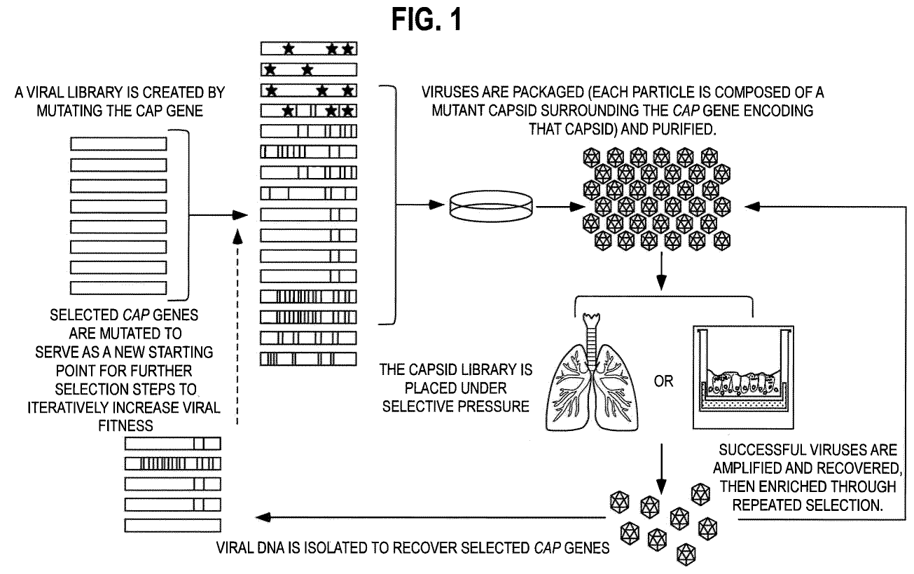

A directed evolution process was applied to discover AAV capsid variants capable of broadly transducing upper airway cells in the primate lung following aerosol administration (FIG. 1). Briefly, a library of approximately 1 billion unique synthetic variant AAV capsid sequences was created from 37 different proprietary sub-libraries using various molecular biology techniques and several different AAV serotypes as templates. The library was packaged in HEK293T cells to produce viral particles such that each virus particle was composed of a synthetic capsid shell surrounding the viral genome encoding that same capsid. Variants within the library were then subjected to in vivo and in vitro selective pressure techniques in NHP and human cell cultures to mimic clinical gene therapy treatment. All synthetic libraries were injected for the first round of selection. After DNA was harvested from lung tissue or cell cultures, the genomes of capsids amplified from the tissue were then packaged as above as the starting library for the next round of selection. This procedure was carried out for a total of five cycles.

Motifs were declared as “Hits” when certain selection criteria were met: (1) a motif represents a certain percentage of the sequenced population in two or more consecutive rounds of the selection or (2) a motif representing a certain percentage of the sequenced population in one or more rounds of the selection

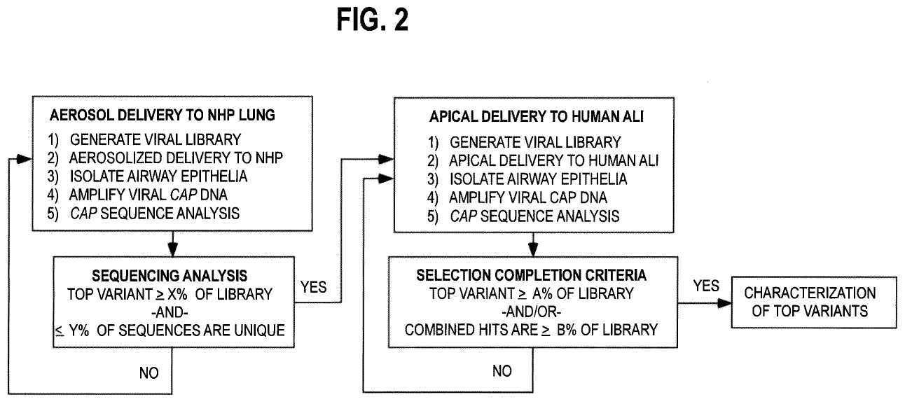

The point at which the model system was transitioned from the in vivo NHP to the in vitro proximal airway organotypic culture was based on the results of the sequencing analysis performed after the second and third rounds of NHP delivery (FIG. 2). Briefly, the transition to the in vitro human ALI model system occurred when: (1) the most frequent Hit represents a certain percentage of the sequenced population and (2) less than a certain percentage of the sequenced clones represent unique sequences

The selection was considered complete when the following Selection Completion Criteria were met: less than two new “Hits” are identified in the round, and (1) the combined “Hits” represent a certain percentage of the sequenced population and/or (2) a single “Hit” represents a certain percentage of the population.

### Cell Lines and Library Production

HEK293T cells were obtained from the American Type Culture Collection (Manassas, VA). Cells were cultured at 37° C. and 5% CO₂ in Dulbecco's modified Eagle's medium supplemented with 10% fetal bovine serum (FBS; Gibco, Carlsbad, CA) and 1% penicillin/streptomycin (Invitrogen, Carlsbad, CA). Viral libraries were produced in HEK293T cells using triple transfection, and viruses were purified by iodixanol gradient centrifugation^{19,21} and Amicon filtration. DNase-resistant genomic titers were determined via quantitative PCR (qPCR), as previously described. 19.21

### Intratracheal Injection and Tissue Harvesting

For each round of selection, a single male cynomolgus macaque (Macaca fascicularis) between 4-6 years of age and weighing between 5.5-7.1 kg was dosed. The animals were anesthetized with 10 mg/kg ketamine and 15 μg/kg dexmedetomidine delivered intramuscularly (IM). Five mL of the library was pre-complexed with 1.75 mg/mL of human intravenous immunoglobulin (IVIG) and administered as described below. Each animal was intubated with a 5 mm endotracheal tube, with the tip of the tube positioned at the level of the clavicle (approximately 5 cm above the carina), and its position confirmed by fluoroscopy. Animals were placed in a chair in a seated position for administration.

For the first round of selection, the nebulizer device was connected to the distal end of the endotracheal tube, and a bird respirator was used to deliver breaths at a rate of 15±1 breaths/minute with a pressure of 20 cm H₂O. For the second and third rounds of selection, an AeroEclipse II nebulizer device was connected to the distal end of the endotracheal tube and a bird respirator was used to deliver breaths at a rate of 12-24 breaths/minute with a pressure of 15-20 cm H₂O. Following the completion of dosing, each animal was extubated and received 0.15 mg/kg atipamezole IM to reverse sedation. The animals were visually monitored until fully recovered from anesthesia prior to returning to their home cages.

Euthanasia was performed by trained veterinary staff using 100 mg/kg pentobarbital sodium delivered intravenously on day 15±1. The lungs, including the trachea, were removed and dissected as detailed below. DNA was isolated from the upper airway cells and stored at −20° C. until viral genome amplification.

### Upper Airway Epithelial Cell Isolation

NHP or human lungs were flushed with phosphate buffered saline (PBS; Gibco, Carlsbad, CA) to remove excess mucosal secretions and residual blood. The trachea and primary, secondary, and tertiary bronchi were isolated away from parenchymal lung tissue. Excess supporting tissue and lymph nodes were removed. Trachea and bronchi were cut into 2-4 cm pieces and placed into an enzymatic solution to relieve epithelial cells (Pronase, 1.4 mg/mL and DNase, 0.1 mg/mL), trachea and primary bronchi were processed together, and secondary and tertiary bronchi Cell solution was incubated for 48 hours at 4° C., with tube inversions twice a day. The enzyme was deactivated by the addition of FBS up to 10% of the total volume. Sections were then cut length wise and epithelial lining was scraped from cartilage. Cells were collected and centrifuged at 300×g for 10 minutes. Cell pellet was rinsed twice with PBS. Pellet was resuspended in Airway Epithelial Cell Basal Medium with supplements (ATCC, Manassas, Virginia) with 5% FBS, and cells were plated onto tissue culture treated 10 cm dishes to allow the fibroblasts to adhere, further purifying the cell isolation to airway epithelial cells. Two to four hours post seeding, media with non-adherent cells were collected and centrifuged at 300×g for 10 minutes. Pelleted cells were lysed to extract library DNA or resuspended in PBS, counted and seeded for in vitro selection rounds.

### In Vitro Library Transduction

Human ALI cultures were transduced 30 days after seeding on human placental collagen, type IV (Sigma). On the day of transduction, three inserts were incubated with Trypsin-EDTA 0.05% (ThermoFisher) for 10 minutes at 37° C. Trypsin was deactivated with Defined Trypsin Inhibitor (ThermoFisher). Cells were collected from the insert and counted on a hemocytometer. An average cell number was determined per insert and used to calculate total viral genomes required per insert. The mucus produced by the cultures was removed prior to viral transduction by washing with PBS. A multiplicity of infection (MOI) of 50,000 for Round 4 and 10,000, 25,000 and 50.000 for Round 5 were used. Each round was run in two parallel sets one where the library was pre-complexed with a 1:10 dilution of human IVIG for 30 minutes prior to cell administration and one without IVIG pre-complexing. Cells were exposed apically for 24 hours with the library. Four days post-infection, DNA was isolated from the cultures and stored at −20° C. until viral genome amplification.

### DNA Quantification, DNA Amplification, and Sequencing Analysis

DNA was isolated from cells described above using the DNeasy Blood & Tissue kit (Qiagen). AAV library genomes were quantified by digital droplet polymerase chain reaction (ddPCR). AAV variant cap genes were amplified by PCR. The cap genes were inserted into the pSub2 library packaging plasmid using NotI and HindIII. Cap genes were then sequenced by third-party DNA sequencing facilities. The sequencing files were analyzed using Geneious software (Biomatters).

### Results

### Pilot Studies for Delivery Device Parameters and Upper Airway Cell Isolation

Two delivery devices, Valley Biosystem's in-house nebulizer and an AeroEclipse II breath actuated nebulizer (Trudell Medical International), were employed to enable downstream compatibility with multiple clinically translatable devices. Both delivery devices were evaluated in pilot studies delivering Evans blue dye to ensure that ventilation parameters resulted in adequate distribution to all lung lobes and the alveolar sacs. Valley Biosystem's in-house nebulizer demonstrated good distribution to all lobes, including the alveolar compartment, with more intense dye observed in the dependent lobes. In parallel to the first round of selection, additional method development and airway delivery device testing was performed to successfully adapt and develop the AeroEclipse II breath actuated nebulizer to deliver aerosolized AAV particles to intubated NHPs. Through a dye distribution study, superior, non-dependent delivery to all six NHP lung lobes, including both cranial lobes were demonstrated. The AeroEclipse II nebulizer was then used for Rounds 2 and 3 of the selection.

The upper airway cell isolation protocol was optimized using a total of 6 NHP lungs. The protocol optimization resulted in high yield and purity of cells from the trachea and primary, secondary, and tertiary bronchi isolated from all three sets of NHP lungs used during Therapeutic Vector Evolution.

### Library Generation & Production

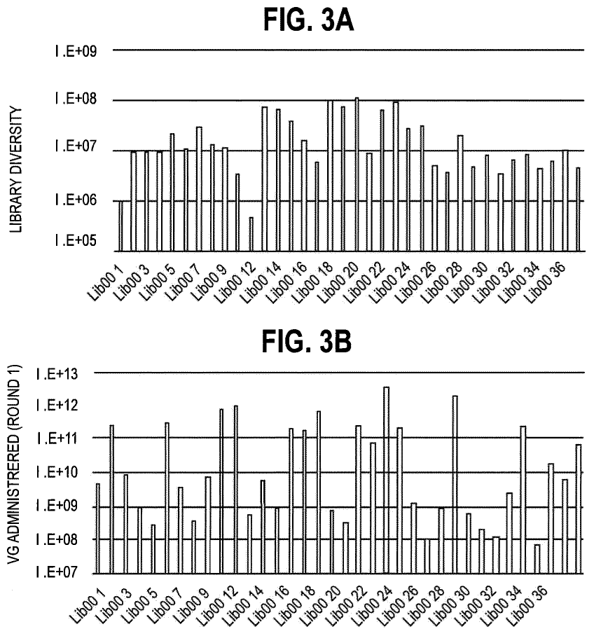

Prior to initiation of Round 1 of the Therapeutic Vector Evolution program, all 37 capsid libraries were synthesized, manufactured, and characterized. As shown in FIG. 3a, the diversity of the plasmid libraries was estimated to include approximately 1×10⁶ to >1×10⁸ unique variants per library, for a total diversity of >1 billion genetic variant sequences. This represents a high quality, highly diverse starting library of AAV variants. Next, production of each individual library was completed in order to generate enough material for the first round of selection. As shown in FIG. 3b, all libraries manufactured at a level sufficient to produce material for an in vivo Therapeutic Vector Evolution selection. Following production and prior to library administration, the sequences from all libraries were evaluated for the presence of non-functional mutations (i.e., frameshift mutations or stop codons) and the frequency of unique sequences by Sanger sequencing. Most libraries had minimal frequency of non-functional mutations, and all libraries were incorporated into the first round of administration to an NHP.

### Aerosol Delivery to NHP Lung

For the first round of selection, all libraries were combined and successfully administered to a single NHP via a single dose aerosol administration using the Valley Biosystems' in-house nebulizer system. Prior to administration, the libraries were incubated with 1.75 mg/mL human IVIG. This represents a high, yet physiologically-relevant lung mucus concentration of human NAbs. The administered library dose of 1.7×10¹² vg represents a dose that is approximately 100-fold lower than the current maximum feasible dose based on manufacturing considerations. Therefore, this represents stringent selective pressure to enable discovery of a vector capable of transducing upper airway cells in the trachea and bronchi. The NHP lungs were harvested two weeks post-administration. Cells were isolated from the lungs in separate trachea/primary bronchi and secondary/tertiary bronchi samples, and DNA was isolated from the cells. High total cell yields and high populations of acetylated tubulin+ cells were obtained from both regions. This process was repeated for two additional rounds using progressively lower doses of viral library.

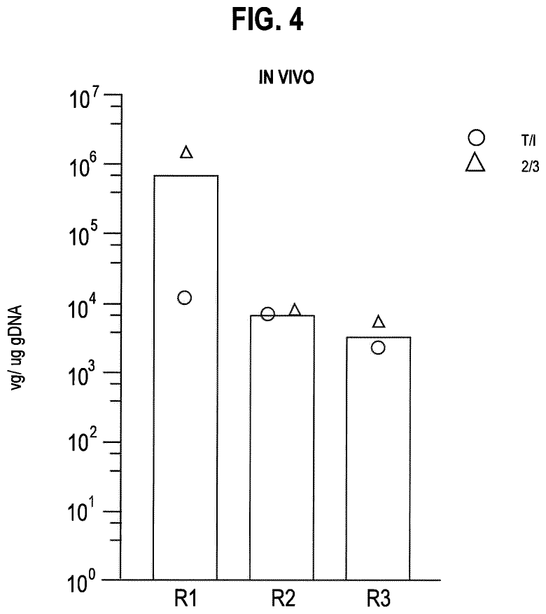

Following DNA isolation from each round of selection, AAV viral library genomes were quantified by ddPCR to confirm successful localization of library vectors to the cell types of interest. There was a dose-dependent decrease in the quantity of viral genomes present in the upper airway, corresponding to the lower doses administered each round (FIG. 4).

In addition to quantification by ddPCR, amplification of capsid genes from tissue represents successful localization of library vectors to the cell type of interest. The capsids amplified from each round of selection in NHPs were cloned into an AAV library packaging plasmid for sequence analysis and to initiate the subsequent round of selection. Sequencing was performed on individual clones within the library to determine the frequency of variants within the population. Sequencing on a minimum of 90 clones from the trachea/primary bronchi and secondary/tertiary bronchi samples for each round was performed. Variants were evaluated for the presence of motifs within the sequencing data. Variants were grouped into motifs based on the presence of a unifying variation (for example, a specific point mutation or specific peptide insertion sequence in a consistent location within the capsid) that occurred in multiple sequences. A motif was nominated as a Hit if it represented a certain percentage of the sequenced population in two or more consecutive rounds of the selection or a certain percentage of the sequenced population in one or more rounds of the selection.

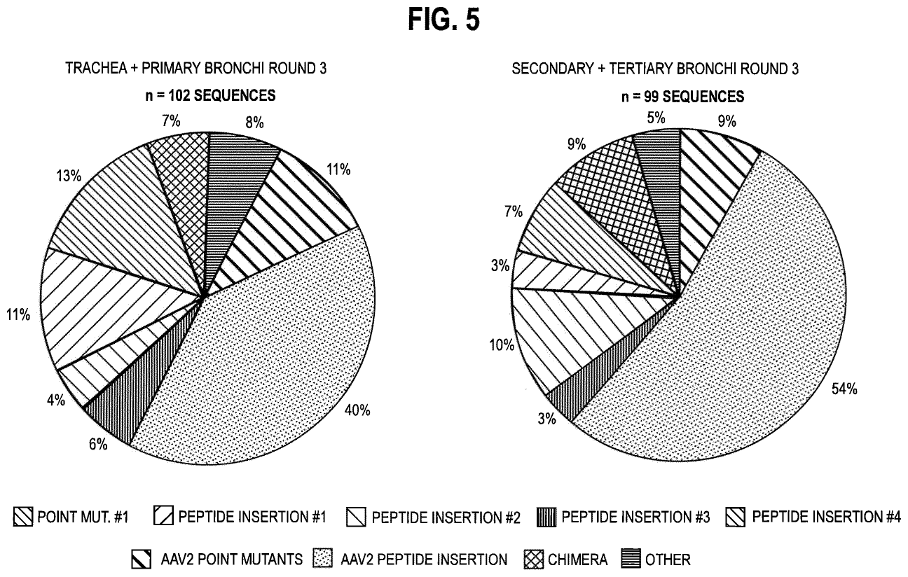

Two motifs (Point Mut. #1. Peptide Insertion #1 (SEQ ID NO:40) within the trachea/primary bronchi region and one motif (Peptide Insertion #2 (SEQ ID NO:41) within the secondary/tertiary bronchi region were nominated as Hits following in vivo Round 3 (FIG. 5). In addition, two more motifs of interest (Peptide Insertion #3 (SEQ ID NO: 42), Peptide Insertion #4 (SEQ ID NO:43) were identified as motifs that would be monitored to determine whether either or both could be nominated as a Hit following further in vitro rounds of Therapeutic Vector Evolution. Point Mut. #1 in the trachea/primary bronchi region and Peptide Insertion #2 in the secondary/tertiary bronchi region were the most frequent hits for each region, and each of these Hits represented a certain percentage of the sequenced AAV population for that region (FIG. 5). Within the total sequenced populations, a certain percentage of the sequenced AAV clones represented unique AAV sequences. Therefore, based on the sequencing analysis performed for Round 3 of the Therapeutic Vector Evolution program, the model system was transitioned from the in vivo NHP to the in vitro proximal airway organotypic culture for the remainder of the selection process.

### Apical Delivery to Human ALI Culture

For in vitro Rounds 4 and 5 of selection, cells from three human lung donors were pooled and cultured in the transwell system in multilayer, air-liquid interface (ALI) culture. The cells formed striated layers and generate mucus. The cell culture composition of the human ex vivo lung epithelial airway culture was assessed 30 days post-thaw by immunocytochemical analysis using antibodies against acetylated tubulin (marker of ciliated cells), cytokeratin 5 (marker of basal cells), and mucin (marker of goblet cells) prior to library transduction.

The AAV libraries were administered to the apical side of the proximal airway organotypic culture system at MOI of 50,000 and 10,000, respectively, for Rounds 4 and 5. Similarly to the in vivo portion of the selection, following DNA isolation from each round in vitro, AAV viral library genomes were quantified by ddPCR to confirm successful localization of library vectors to the cell types of interest. Unlike in the in vivo portion of the selection, no dose-dependence was observed between rounds (FIG. 6a), likely due to the high MOIs used in vitro. Furthermore, pre-incubation with human IVIG did not appear to have a significant impact on genome presence following transduction (FIG. 6b).

As before, amplification of capsid genes from tissue represents successful localization of library vectors to the cell type of interest. The capsids amplified from each round of selection in vitro were again cloned into an AAV library packaging plasmid for sequence analysis and to initiate the subsequent round of selection. Sequencing was again performed on individual clones within the library to determine the frequency of variants within the population. Sequencing on a minimum of 89 clones from the conditions in the absence and presence of human IVIG for each round was performed.

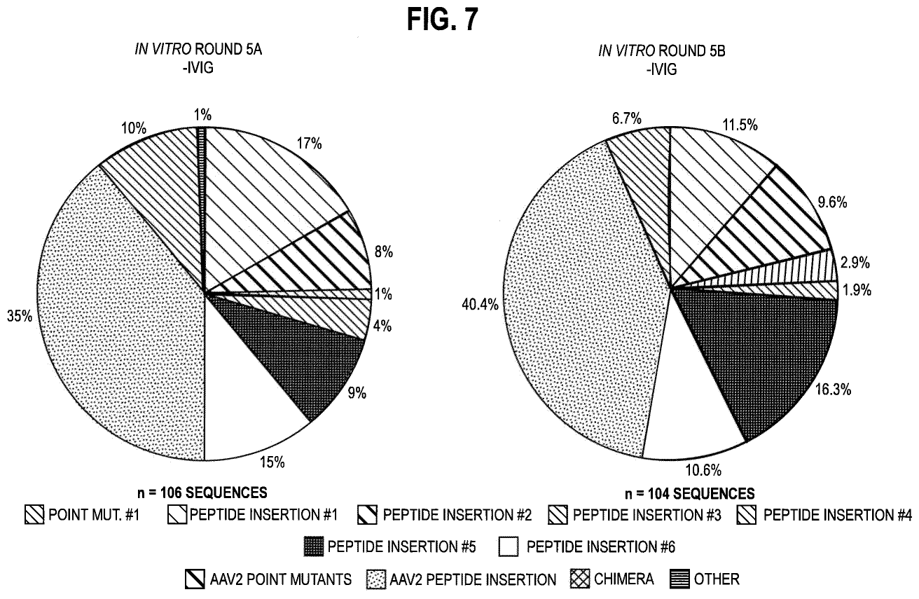

The Hits identified during the in vivo portion of the selection (Point Mut. #1, Peptide Insertion #1, Peptide Insertion #2) remained at a relatively high frequency following two rounds of in vitro selection (FIG. 7). One of the variants of interest (Peptide Insertion #3) decreased in frequency and never met the criteria to be nominated as a Hit. The other variant being monitored following Round 3 (Peptide Insertion #4) did meet the criteria to be nominated as a Hit following Round 4, but it also decreased in frequency in Round 5 (FIG. 7). Following Round 4, two additional motifs (Peptide Insertion #5 (SEQ ID NO:44), Peptide Insertion #6 (SEQ ID NO:45) were nominated as Hits. No new Hits were identified in Round 5, and the combined Hits represented greater than 50% of the sequenced population (FIG. 7). Therefore, based on the sequencing analysis performed for Round 5 of the Therapeutic Vector Evolution program, the discovery program was considered complete.

### Results

AAV capsids were successfully amplified from isolated, upper airway cell populations isolated from the trachea and primary, secondary, and tertiary bronchi from three sequential rounds of selection in NHPs following a single round of intratracheal aerosol administration of library using a nebulizer. Human in vitro ALI cultures were utilized for two subsequent rounds of selection. Following successful amplification of viral genomes from NHP lungs or human ALI cultures, sequencing was performed on individual clones within the library. Individual sequences were grouped into motifs based on the presence of a unifying variation and evaluated based on frequency within the sequencing analysis and diversity of variations. Following analysis, six variant sequences emerged as hits that were nominated for further characterization. These six variants (AAV102-AAV107, SEQ ID Nos: 68-73) are AAV2-based capsids, each of which contains a peptide inserted in a loop region and a V708I amino acid substitution.

A summary of the frequency of peptide insertion motifs for each of the selection rounds described above is provided below at Table 1:

TABLE 1

| | SEQ | Round | Round | Round | Round | Round | Round | Round | Round | Round | Round |
|---|---|---|---|---|---|---|---|---|---|---|---|
| Peptide | ID | 1 | 1 | 2 | 2 | 3 | 3 | 4 (+ | 4 (− | 5 (+ | 5 (− |
| Insertion | NO: | T/1° | 2/3° | T/1° | 2/3° | T/1° | 2/3° | IVIG) | IVIG) | IVIG) | IVIG) |
| LAHDITKNIA | 40 | | | | | 10.78% | 3.03% | 9.7% | 10.8% | 11.7% | 17.1% |
| LANQDYTKTA | 41 | | | 0.54% | | 3.92% | 10.10% | 6.8% | 8.9% | 9.7% | 7.6% |
| LADNTVTRSA | 42 | | | 1.63% | | 5.88% | 3.03% | 0.5% | 2.3% | 2.9% | 1.08 |
| LASNSVQSIA | 43 | | | 0.54% | | 0.98% | 7.07% | 6.3% | 4.2% | 4.9% | 3.8% |
| LANSTRHTDA | 44 | | | | | 0.98% | 3.03% | 10.6% | 4.7% | 16.5% | 9.5% |
| LATNRTSPDA | 45 | | | 0.54% | | | 1.01% | 10.1% | 9.9% | 10.7% | 15.2% |
| LAISDQTKHA | 46 | | 1.10% | 0.54% | | 0.98% | 4.04% | | 1.9% | 2.9% | 1.0% |
| LAQADTTKNA | 47 | | 1.10% | | | 2.94% | 2.02% | 2.4% | 3.3% | 1.0% | 2.9% |
| LANAVKTDEA | 48 | | | | | 0.98% | | 1.9% | 3.8% | 2.9% | 2.9% |
| LATNQTLSAA | 49 | | | | | 1.96% | 1.01% | 3.9% | 3.3% | 3.9% | 1.9% |
| LAENRTTSNA | 50 | | | | | 3.92% | 2.02% | 3.4% | 3.3% | | 3.8% |
| LAPQQDTTHA | 51 | | | | | 0.98% | 4.04% | 2.4% | 3.3% | 4.9% | 1.0% |
| LANATNHVIA | 52 | | | | | 1.96% | | 1.0% | 2.3% | | 1.9% |
| LATNNSKPDA | 53 | | | | | | | 1.4% | 2.3% | 1.0% | |
| LASKLTLNNA | 54 | | | 0.54% | | | 1.01% | 0.5% | 0.5% | | 1.0% |
| LAVTAGMGAA | 55 | | | | | | 3.03% | 1.9% | 0.9% | | |
| LAPNSTTNNA | 56 | | | 0.54% | | 0.98% | 1.01% | 0.5% | 1.9% | | |
| LANSTSRIDA | 57 | | | | | | 2.02% | 3.4% | 1.4% | 3.9% | 2.9% |
| LAVASHTNNA | 58 | | | 1.09% | | | 4.04% | 0.5% | 0.5% | | 1.0% |
| LARSHQEIPA | 59 | | | | | 1.96% | 1.01% | 0.5% | 1.4% | 1.0% | 1.0% |
| LALNTTKDIA | 60 | | | | | | 2.02% | 1.0% | 0.9% | 4.9% | |
| LAIIDATKNA | 61 | | | | | 0.98% | 1.01% | 0.5% | 1.9% | 1.0% | 4.8% |
| LANHISQTNA | 62 | | | 0.54% | | 2.94% | | 1.4% | 0.5% | 1.0% | |
| LASNSAHITA | 63 | | | | | 1.96% | | 0.5% | 1.4% | | |
| LASTHQSNNA | 64 | | | 1.09% | | 0.98% | 4.04% | 1.0% | 0.5% | | |
| LAKTPNLTSA | 65 | | | 1.09% | | 2.94% | | 1.0% | 0.9% | | 1.0% |
| LASNTPALSA | 66 | | | 0.54% | | 0.98% | 1.01% | 1.4% | 1.4% | | 1.9% |
| LASPGATTNA | 67 | | | | | | | 1.4% | 0.9% | 1.0% | |

T/1º = trachea/primary bronchi
2/3º = secondary/tertiary bronchi
IVIG = human intravenous immunoglobulin (1:10)

Sequenced clones identified from the directed evolution screen included variant capsids with only a peptide insertion (e.g., of SEQ ID No: 40, 41, 42, 43, 44 or 45) and that are otherwise identical to SEQ ID NO:2 as well as variant capsids with a peptide insert in combination with a variety of amino acid substitutions. A summary of amino acid substitutions (numbering relative to SEQ ID NO:2) that were tolerated in combination with insertion peptides of Table 1 in sequenced clones identified from the directed evolution screen is provided at Table 2 (in each case, the variant AAV capsid protein is otherwise identical to SEQ ID NO:2):

TABLE 2

| | SEQ ID | |
|---|---|---|
| Insertion | NO | Substitution(s) |
| LAHDITKNIA | 40 | V708I |
| LAHDITKNIA | 40 | E99D + V708I |
| LAHDITKNIA | 40 | E347D |
| LAHDITKNIA | 40 | K532E + V708I |
| LAHDITKNIA | 40 | D283E + V708I |
| LAHDITKNIA | 40 | G18E + G220R + |
| | | V708I |
| LAHDITKNIA | 40 | T138R |
| LAHDITKNIA | 40 | R37L + H38Q + |
| | | V708I |
| LANQDYTKTA | 41 | V708I |
| LANQDYTKTA | 41 | N449D + |
| | | D472N + V708I |
| LANQDYTKTA | 41 | P436H |
| LANQDYTKTA | 41 | K544E |
| LANQDYTKTA | 41 | K532E + V708I |
| LANQDYTKTA | 41 | P250S + V708I |
| LANQDYTKTA | 41 | S109T + |
| | | N312K + |
| | | H641N + V708I |
| LANQDYTKTA | 41 | N705S + V708I |
| LANQDYTKTA | 41 | K532E + |
| | | K688R + V708I |
| LANQDYTKTA | 41 | P451Q + |
| | | A593E + V708I |
| LASNSVQSIA | 43 | D213Y + V708I |
| LASNSVQSIA | 43 | T200I |
| LASNSVQSIA | 43 | T344S + E347D |
| LASNSVQSIA | 43 | V708I |
| LADNTVTRSA | 42 | V708I |
| LANSTRHTDA | 44 | L91I |
| LANSTRHTDA | 44 | P30L + A593E + |
| | | V708I |
| LANSTRHTDA | 44 | P250S + V708I |
| LANSTRHTDA | 44 | V708I |
| LATNQTLSAA | 49 | R103C + V708I |
| LATNQTLSAA | 49 | V118A + V708I |
| LAPQQDTTHA | 51 | S109T + V708I |
| LAPNSTTNNA | 56 | S16Y + V708I |
| LAPQQDTTHA | 51 | N469D + V708I |
| LATNRTSPDA | 45 | A591D + V708I |
| LATNRTSPDA | 45 | V708I |
| LAQADTTKNA | 47 | S109T |
| LAQADTTKNA | 47 | R585K |
| LAQADTTKNA | 47 | R585K + V708I |
| LAQADTTKNA | 47 | R103L + |
| | | K532E |
| LAQADTTKNA | 47 | E133D |
| LAQADTTKNA | 47 | T491I + K532E |
| LAQADTTKNA | 47 | R585K + V708I |
| LANAVKTDFA | 48 | D608N + V708I |
| LANAVKTDFA | 48 | Q120H + V708I |
| LAENRTTSNA | 50 | V708I |
| LAENRTTSNA | 50 | T138R |
| LAENRTTSNA | 50 | S109T |
| LAENRTTSNA | 50 | S109T + V708I |
| LARSHQEIPA | 59 | G376A + V708I |
| LAIIDATKNA | 61 | T138R + |
| | | G376A + |
| | | Q428H + V708I |
| LATNNSKPDA | 53 | K532E |
| LATNNSKPDA | 53 | V118A + |
| | | M371I |
| LANSTSRIDA | 57 | K532E + V708I |
| LASPGATTNA | 67 | Y6F |
| LAISDQTKHA | 46 | E134Q + |
| | | P135A + |
| | | V136G + |
| | | K137E + V708I |

### Example 2

The six variant capsid sequences identified above (AAV102-AAV107; SEQ ID Nos: 68-73), exhibiting a preference toward upper airway epithelial cell transduction, were characterized in vitro in non-human primate (NHP) and human ex vivo lung upper airway air-liquid interface (ALI) cultures at a range of multiplicity of infections (MOIs).

Briefly, recombinant AAV (rAAV) was manufactured, each comprising a cap variant sequence of AAV102-AAV107 containing a reporter cassette, with a ubiquitous promoter (CMV enhancer, chicken beta-actin promoter and rabbit beta-globin splice acceptor site, CAG) driving EGFP. Transduction of the six variant AAV capsids, AAV101, and natural serotypes AAV2 and AAV5 was assessed in vitro at four multiplicity of infections, 12,500, 25,000, 50,000 and 100,000.

A first-generation capsid variant (AAV101) engineered for enhanced transduction of airway epithelia in organotypic ALI cultures in vitro has been shown to be superior to naturally occurring AAV capsids (see SEQ ID NO:12 of U.S. Pat. Publ. No. 2021/0395772, the contents of which are incorporated herein by reference). However, additional discovery was undertaken to identify vectors that could efficiently transduce both in vitro and in vivo at a level sufficient to achieve clinical benefit in cystic fibrosis (CF).

### Methods

### AAV Manufacturing

rAAV comprising each of variant capsid proteins AAV101-AAV107 and natural serotypes AAV2 and AAV5 were produced with a triple plasmid transfection process which utilized a commercially available transfection reagent and Human Embryonic Kidney 293 (HEK293) cells cultured in flatware. Transfected cells were harvested along with supernatant, and, subsequently, nuclease treated and clarified through a 0.2 μm filter. The clarified harvest was purified using affinity chromatography (AVB Sepharose HP, Cytiva Life Sciences) and buffer exchanged into DPBS with 0.005% Pluronic F68. After 0.2 μm filtration the bulk drug substance (BDS) was aliquoted into cryovials (Corning) and stored at −80° C.

Digital Droplet PCR (ddPCR) for AAV Titer Analysis

The viral genomic titer is determined by ddPCR. The test sample is diluted, and DNase treated, then further diluted into DPBS with 0.02% Pluronic F68, ddPCR Supermix, and primers/FAM-labeled probes corresponding to the SV40Poly A sequence. 20 μL samples are partitioned into droplets using a Bio-Rad Automated Droplet Generator, subjected to PCR, then read on the Bio-Rad QX200 Droplet Reader, which measures each droplet individually for fluorescent signal. Data is analyzed using Bio-Rad QuantaSoft software, which uses Poisson statistical analysis of positive and negative droplets to provide absolute quantitation of target sequence. No-template controls are used to set the negative baseline for samples. An internal ddPCR reference standard virus is used as the reference standard control.

### Upper Airway Epithelial Cell Isolation and Culture

Airway epithelial cells were isolated from the trachea and primary through tertiary bronchi of NHP and human donor lungs that were rejected from transplant (Donor Network West) following a published protocol (Karp et al., 2002). Isolated cells were frozen and stored in liquid nitrogen.

Airway epithelial cells were thawed onto human placental collagen IV (60 μg/mL, MilliporeSigma, Burlington, MA) in Airway Epithelial Cell Basal media with growth supplements in the Bronchial Epithelial Cell Growth Kit (ATCC, Manassas, VA) onto transwell inserts (0.4 μm, 0.32 cm², Corning, Corning, NY). Two days after seeding, the media was aspirated from the insert and basal chamber. The basal chamber was replenished with PneumaCult ALI Basal media with the growth supplement provided (Stem Cell Technologies, Vancouver, Canada). Basal media was changed every 2-3 days. Liquid in the apical side of the transwell was aspirated every day until air liquid interface (ALI) was achieved. Cells were matured for 30 days prior to cell characterization and transduction.

### AAV Transduction

Following at least 30 days in culture, cells were extensively washed to remove built up mucus. Three inserts of each species were harvested, and cells counted. Based on AAV titer and the average cell count for each species, the volume of virus was diluted to reach a MOI of 12,500, 25,000, 50,000 and 100,000. Volume was equalized with diluent to yield a 100 μl transduction volume per insert for each species. Human cells were apically and basally transduced, and NHP cells were apically transduced with AAV following mucus wash, n=1 per condition. Liquid on the insert was aspirated 24 hours post infection and basal compartment media was exchanged. Cells were fixed for immunocytochemistry seven days post infection.

### Immunocytochemistry

Cells were fixed with 4% paraformaldehyde for 20 minutes at room temperature. Cells were then imaged using a Zeiss Axio Observer D.1 fluorescent microscope to visualize EGFP looking top down on the cells. Inserts containing the cells were then embedded in optimal cutting temperature media (OCT, ThermoFisher, Waltham, MA) and sectioned in 20 μm slices on the CryoStar cryostat. Slides containing cross sections of inserts were blocked in 2% goat serum, 5% bovine serum albumin in 0.02% triton X-100 in PBS for 1 hour at room temperature. Slides were then incubated with primary antibodies for 2 hours at room temperature, washed three times with 0.02% triton X-100 and incubated with secondary antibodies for one hour at room temperature. Slides were then counterstained with DAPI (nuclei stain) for five minutes at room temperature, washed three times and sealed with a cover slip using Prolong Gold Anti-fade mountant (ThermoFisher). Cells were then imaged using a Zeiss Axio Observer D.1 fluorescent microscope.

Digital Droplet Polymerase Chain Reaction (ddPCR) for AAV Transcript Detection

RNA was extracted from each insert using the Qiagen RNeasy Plus Mini Kit according to manufacturer's instructions (Qiagen, Hilden, Germany). RNA was quantified and 2 μg of cDNA was generated using the iScript cDNA Synthesis Kit according to manufacturer's instructions (40 μL total volume, Bio-Rad, Hercules, CA). Dilutions of cDNA were made at 1:10 and 1:100 in water to a total volume of 50 μL, 2 μL was added per assay plate well. Master mixes were made of 2× Supermix ddPCR™ (no dUTPs, Bio-Rad) and ddPCR compatible primers/probes mixes (HPRT1 HEK-housekeeper, 20× ID Bio-Rad and a SV40 FAM ID (Furuta-Hanawa, Yamaguchi, & Uchida, 2019)) up to a total volume 18 μL per well. SV40 primers/probes are a surrogate for measuring EGFP transgene transcript levels, as an SV40 polyadenylation site is included as part of the virally delivered transgene cassette. Mastermix and cDNA were combined in a ddPCR™ 96-well assay plate and loaded into the QX200™ AutoDG Droplet Digital™ PCR System according to the manufacturer's instructions. The droplets were generated, and the plate was transferred to C1000 Touch Thermal Cycler to run PCR. ThermoCycler conditions were as follows: 94° C. 10 minutes, 94° C. 30 seconds, 60° C. 1 minute (Repeat steps 2-3, 39×), 98° C. 10 minutes, hold at 12° C. The plate was then transferred to the QX200™ Droplet Reader with parameters set to read in the FAM and HEK channels. Data was analyzed in the QuantaSoft software with a threshold of 3000 and then transferred to an Excel file where further analysis was completed. A graph was created using a ratio of SV40 to housekeeper values, using the lowest dilution with a viable read.

### Mean Fluorescence Intensity Calculation

For each well of ALI transduced, 3 images were taken per well using a 10× objective using a Zeiss Axio Observer D.1 fluorescent microscope. The regions imaged were chosen in a consistent and unbiased pattern from well to well. The exposure time for each image was set using the standard AAV2 MOI 25,000 transduction wells. After imaging, all images were processed using the ImageJ software. Background fluorescence was subtracted and then the “Measure Mean Gray Value” tool was used to produce MFI values. An average was taken of the three images from each well to find the final MFI value for each well. Each well within a condition was then averaged and a standard deviation calculated to produce the final graph (FIG. 3b).

### Results

### AAV Manufacturing of Novel Capsids

rAAV comprising the variant capsid proteins and wildtype serotypes were manufactured by triple transfection in HEK293 cells cultured on CellSTACKs and purified using affinity chromatography. Upstream productivities (reported in units of log viral genomes (vg) per square centimeter of cell culture surface area) were measured after supernatant and cell harvest by ddPCR analysis (Table 1, Upstream Productivity). All novel capsids were able to package the GFP payload and showed comparable productivities to other AAV vectors produced using a flatware production process. Downstream yield (reported as % of vgs recovered) was determined by ddPCR following affinity purification and buffer exchange (Table 3, Downstream Yield). Initial packaging and yield data for AAV101-EGFP is also shown in Table 3 for productivity and yield comparison.

TABLE 3

| | | | | Downstream |
|---|---|---|---|---|
| | | | Upstream | Yield |
| | | Amino Acid | Productivity | (% vgs |
| Vector | Peptide Insert* | Substitution* | (log vg/cm2) | recovered) |
| AAV101- | N/A | N/A | 9.97 | 42% |
| EGFP | | | | |
| AAV102- | LAHDITKNIA | V708I | 9.95 | 46% |
| EGFP | (SEQ ID NO: 40) | | | |
| AAV103- | LANQDYTKTA | V708I | 10.02 | 40% |
| EGFP | (SEQ ID NO: 41) | | | |
| AAV104- | LADNTVTRSA | V708I | 10.02 | 61% |
| EGFP | (SEQ ID NO: 42) | | | |
| AAV105- | LASNSVQSIA | V708I | 9.82 | 56% |
| EGFP | (SEQ ID NO: 43) | | | |
| AAV106- | LANSTRHTDA | V708I | 10.01 | 57% |
| EGFP | (SEQ ID NO: 44) | | | |
| AAV107- | LATNRTSPDA | V708I | 9.88 | 52% |
| EGFP | (SEQ ID NO: 45) | | | |

*relative to AAV2 capsid of SEQ ID NO: 2

### Transduction Efficiency of AAV Novel Variants

Transduction efficiency of the six novel variants identified in Example 1 were compared to AAV controls: AAV2, AAV5 and AAV101. Each capsid contained a cassette with a ubiquitous promoter (CAG) driving EGFP. Human and NHP upper airway epithelial cells were transduced following removal of mucus. Seven days post infection, cells were imaged top down using an epifluorescence microscope to visualize EGFP.

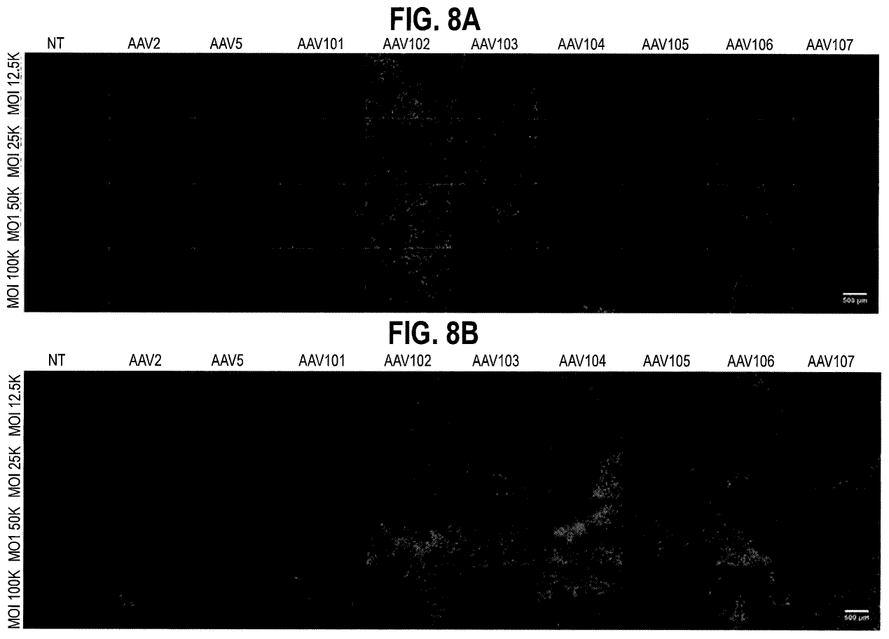

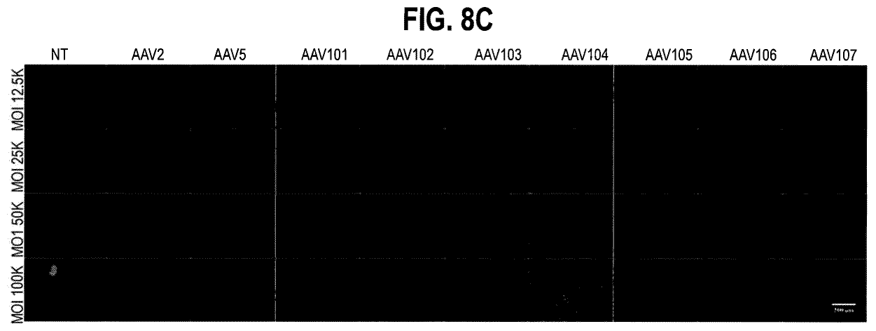

For apically transduced human cells, minimal EGFP expression was detected in AAV2 and AAV5 controls. Of the six novel variants, AAV102 and AAV103 transduction efficiency was greater, at lower doses, than AAV2, AAV5 and AAV101 as analyzed by fluorescent microscopy (FIG. 8a). AAV103 transduction at a MOI 100,000 was low, possibly due to inefficient mucosal washing of that well. AAV106 transduction was higher than AAV2 and AAV5.

In apically transduced NHP cells, EGFP patterns were variable intrawell. Enhanced transduction around the outside edge of the transwell was repeatedly observed, in a ring pattern with limited transduction in the middle of the transwell. Overall, the novel variants showed stronger transduction efficiency than AAV2, AAV5 and AAV101. AAV102 and AAV104 expressed the highest levels of EGFP (FIG. 8b). NHP transduction patterns were not as apparent as in the human cultures, perhaps due to the edge effect. All images shown for the NHP transduction capture a part of the edge of the well to visualize the transduction.

For basally transduced human cells, different patterns emerged. AAV104 had the highest transduction efficiency, greater than AAV2, AAV5 and AAV101 (FIG. 8c). AAV102 and AAV103 did not show superior transduction as they did when apically administered. Overall, the basal transduction appeared lower than the apical transduction. The relative lack of transduction following basal administration compared to apical administration is not surprising, given that these capsid variants were selected through apical administration to NHP in vivo and human ALI in vivo. NHP in vitro cultures were not transduced basally.

The cell culture composition of the human ex vivo lung epithelial airway culture was assessed 30 days post-thaw by immunocytochemical analysis using antibodies against acetylated tubulin (Ac-tub; ciliated cells), cytokeratin 5 (KRT5; basal cells), and mucin (MUC5ac; goblet cells). Cross section analysis of human apically and basally transduced cells were completed and yielded non-conclusive results about specific cell variant tropism within this ALI system (data not shown). More analysis is required to properly understand specific tropism of each capsid within this heterogenous culture system. Additional studies in vivo may also help to better characterize cell-specific transduction for each capsid.

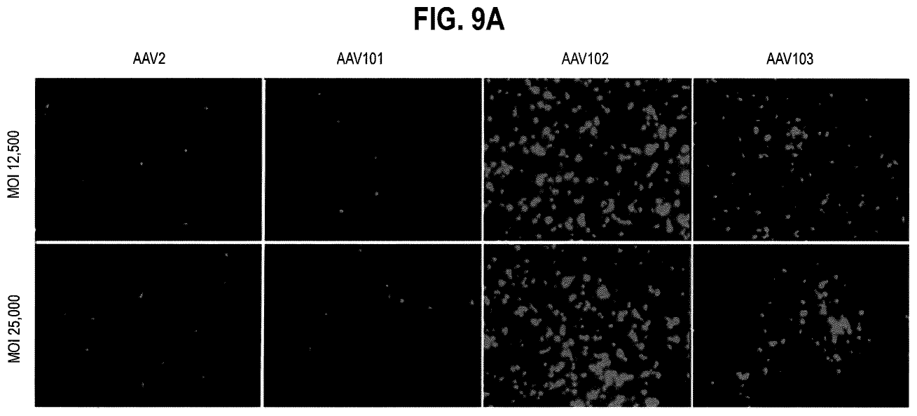

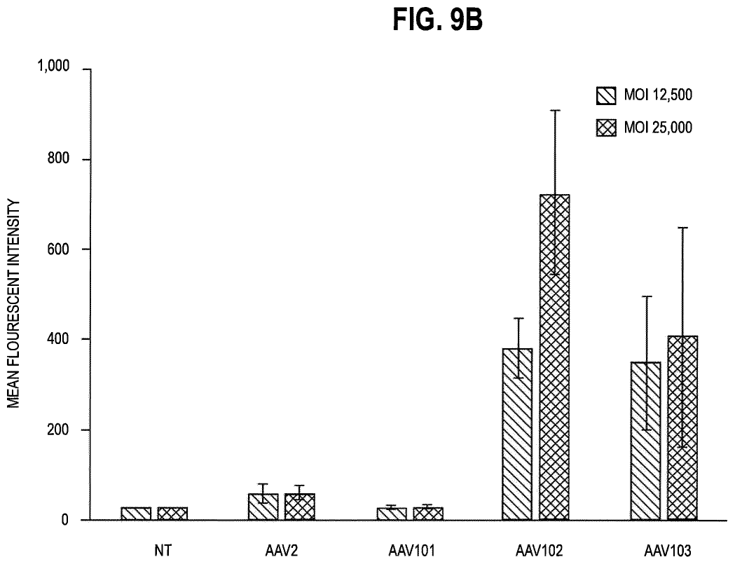

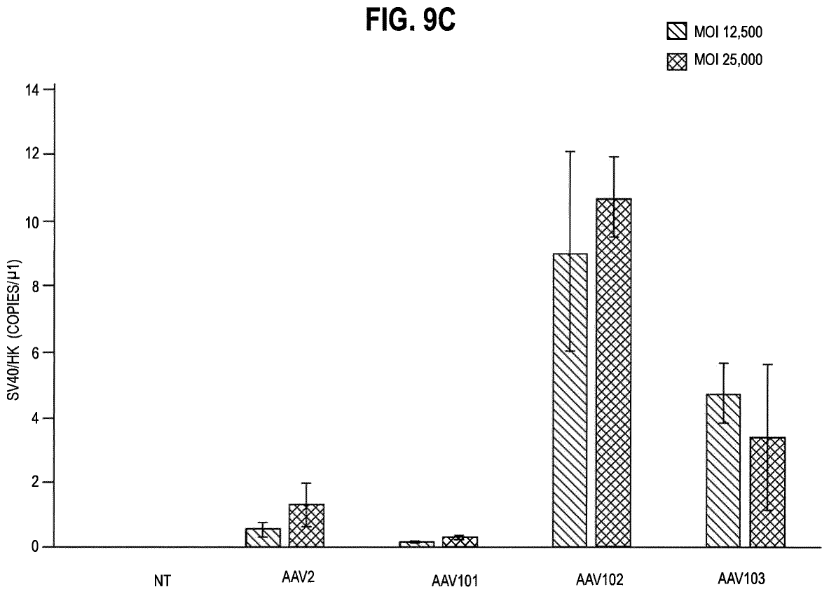

A follow-up study was completed to further examine AAV102 and AAV103, the top performing variants from the initial apical transduction screen in human ALI cultures, compared to AAV2 and AAV101. Immunocytochemistry analysis depicting EGFP is shown in FIG. 9a. The mean fluorescent intensity was averaged and graphed from data of three images per well, three wells per condition (FIG. 9b). Finally, digital droplet PCR determined transcript levels for each variant (FIG. 9c). All three analyses confirmed AAV102 and AAV103 superiority to AAV2 in transduction efficiency of ex vivo airway cultures when transduced apically at low MOIs.

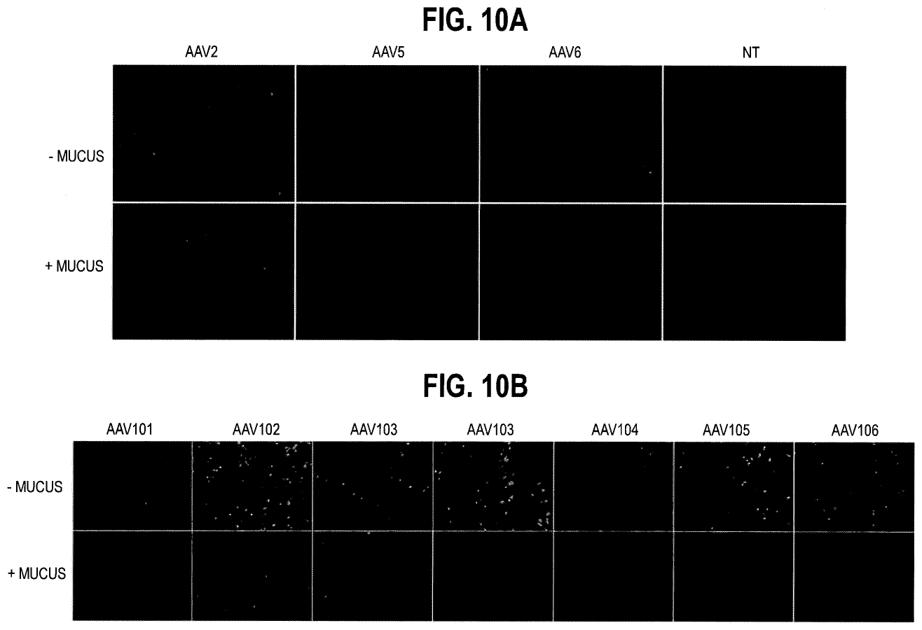

An additional follow-up study was completed to compare apical transduction of human airway epithelial cells in ALI cultures by each of AAV102-AAV107 compared to AAV2, AAV5, AAV6 and AAV101, in the presence and absence of mucus. Briefly, human lung epithelial cell cultures were grown in air-liquid interface for at least 30 days. Cells were transduced (MOI=25.000) with AAV102-AAV107 or control capsid, carrying an EGFP reporter gene driven by a CAG promoter, in the presence or absence of mucus. Seven days post transduction, images of EGFP were taken as a representative of transduction efficiency of the capsid. Each of capsids AAV102-AAV107 demonstrated stronger transduction efficiency compared to wild type AAV serotypes and compared to AAV101 control when transduced in the absence of mucus (FIGS. 10A and 10B, top panels). Transduction in the presence of muscus decreased transduction efficiency of each capsid (except AAV5 which did not show transduction in the absence of mucus) (compare top panels of FIGS. 10A and 10B to bottom panels).

In vitro analysis of six of the top AAV capsid variants identified through selection in NHP using an aerosolized delivery technique and apical administration to human ex vivo upper airway epithelial ALI cultures according to Example 1 demonstrates that rAAV with the variant capsid proteins are superior in transducing human and NHP ex vivo upper airway epithelial ALI cultures compared to AAV101 and wild type AAV serotypes. Different variants demonstrated preference towards transduction depending on route of administration. AAV102 and AAV103 apically transduced human in vitro upper airway epithelial ALI cultures better than AAV101 and WT serotypes at lower MOIs. In basally transduced human cells, AAV104 had the highest transduction efficiency.

### Example 3

The novel AAV capsid variants are examined in a neutralizing antibody screen and an in vivo vector characterization in NHP.

While the materials and methods of this invention have been described in terms of preferred embodiments, it will be apparent to those of skill in the art that variations may be applied to the method described herein without departing from the concept, spirit and scope of the invention. All such similar substitutes and modifications apparent to those skilled in the art are deemed to be within the spirit, scope and concept of the invention.

1. A variant adeno-associated virus (AAV) capsid protein comprising a peptide insertion relative to a corresponding parental AAV capsid protein, wherein the peptide insertion comprises an amino acid sequence selected from DNTVTRS (SEQ ID NO:14), SNSVQSI (SEQ ID NO: 15), and NSTRHTD (SEQ ID NO:16).

2. The variant AAV capsid protein according to claim 1, wherein the insertion site is located within the GH-loop of the capsid protein.

3-4. (canceled)

5. The variant AAV capsid protein according to claim 2, wherein the peptide insertion comprises an amino acid sequence selected from LADNTVTRSA (SEQ ID NO: 42), LASNSVQSIA (SEQ ID NO:43), and LANSTRHTDA (SEQ ID NO:44).

6-10. (canceled)

11. The variant AAV capsid protein according to claim 1, wherein the AAV capsid protein comprises the amino acid sequence set forth in any one of SEQ ID Nos: 70-72.

12. The variant AAV capsid according to claim 1, wherein the capsid protein confers to an infectious rAAV virion an increased infectivity of a lung cell compared to the infectivity of the lung cell by an AAV virion comprising a wild-type AAV capsid protein.

13. (canceled)

14. An isolated nucleic acid comprising a nucleotide sequence that encodes a variant AAV capsid protein according to claim 1.

15. An infectious recombinant AAV (rAAV) virion comprising a variant AAV capsid protein according to claim 1.

16. The rAAV virion according to claim 15 further comprising a heterologous nucleic acid comprising a nucleotide sequence encoding one or more gene products, wherein the nucleotide sequence encoding one or more gene products is operably linked to an expression control sequence.

17-20. (canceled)

21. The rAAV according to claim 16, wherein the heterologous nucleic acid comprises a nucleotide sequence encoding CFTR or a biologically active portion thereof.

22. A host cell comprising the rAAV according to claim 1.

23. A pharmaceutical composition comprising the rAAV according to claim 16 and a pharmaceutically acceptable carrier.

24. A method of delivering a heterologous nucleic acid to a lung cell comprising contacting the lung cell with an rAAV virion comprising (i) a variant capsid protein comprising a peptide insertion selected from the group consisting of HDITKNI (SEQ ID NO:12), NQDYTKT (SEQ ID NO:13), DNTVTRS (SEQ ID NO:14), SNSVQSI (SEQ ID NO:15), NSTRHTD (SEQ ID NO: 16), TNRTSPD (SEQ ID NO:17), LAHDITKNIA (SEQ ID NO:40), LANQDYTKTA (SEQ ID NO:41), LADNTVTRSA (SEQ ID NO:42), LASNSVQSIA (SEQ ID NO:43), LANSTRHTDA (SEQ ID NO:44), LATNRTSPDA (SEQ ID NO:45) and (ii) a heterologous nucleic acid comprising a nucleotide sequence encoding one or more gene products, preferably wherein the rAAV virion is administered to a mammalian subject by pulmonary, endobronchial, intranasal, intratracheal and/or intrabronchial administration.

25. (canceled)

26. The method according to claim 24, wherein the peptide insertion comprises an amino acid sequence selected from LAHDITKNIA (SEQ ID NO:40), LANQDYTKTA (SEQ ID NO:41), LADNTVTRSA (SEQ ID NO:42), LASNSVQSIA (SEQ ID NO: 43), LANSTRHTDA (SEQ ID NO:44), LATNRTSPDA (SEQ ID NO:45).

27-31. (canceled)

32. The method according to claim 24, wherein the variant AAV capsid protein comprises or consists of the amino acid sequence set forth in any one of SEQ ID Nos: 70-72.

33. (canceled)

34. A method for treating a pulmonary disease comprising administering to a subject in need thereof a therapeutically effective amount of an rAAV virion comprising (i) a variant capsid protein comprising a peptide insertion selected from the group consisting of HDITKNI (SEQ ID NO:12), NQDYTKT (SEQ ID NO:13), DNTVTRS (SEQ ID NO:14), SNSVQSI (SEQ ID NO: 15), NSTRHTD (SEQ ID NO:16), TNRTSPD (SEQ ID NO:17), LAHDITKNIA (SEQ ID NO: 40), LANQDYTKTA (SEQ ID NO:41), LADNTVTRSA (SEQ ID NO:42), LASNSVQSIA (SEQ ID NO:43), LANSTRHTDA (SEQ ID NO:44), LATNRTSPDA (SEQ ID NO: 45) and (ii) a heterologous nucleic acid comprising a nucleotide sequence encoding one or more gene products, or a pharmaceutical composition comprising the rAAV virion and a pharmaceutically acceptable carrier

35. The method according to claim 34, wherein the pulmonary disease is selected from chronic obstructive pulmonary disease (COPD), idiopathic pulmonary fibrosis (IPF), cystic fibrosis, pulmonary arterial hypertension, pulmonary hypertension, lung cancer (primary, secondary and metastatic), surfactant deficiency, viral and/or bacterial infection, acute bronchitis, pneumonia (including viral, bacterial, and fungal pneumonia), respiratory tract infections (including pharyngitis, croup, aspergillus, coccidiomycosis, hantavirus pulmonary syndrome, and histoplasmosis), chemical and hypersensitivity pneumonitis, tuberculosis and other mycobacterial infections (including but not limited to Mycobacterium avium), sarcoidosis, respiratory syncytial virus, pulmonary edema, acute respiratory distress syndrome (ARDS), pneumoconiosis (including black lung disease, asbestosis, and silicosis), interstitial lung disease (including sarcoidosis and autoimmune disease), pulmonary embolism, pleural effusion, pleuritis, mesothelioma, pneumothorax, acute bronchitis, bronchiolitis (including bronchiolitis obliterans), sudden infant death syndrome, sleep apnea, bronchiectasis, bronchopulmonary dysplasia, cryptogenic organizing pneumonia, E-cigarette or vaping use associated lung injury (EVALI), Middle Eastern Respiratory Syndrome (MERS), primary ciliary dyskinesia, Severe Acute Respiratory Syndrome (SARS), alpha-1-antitrypsin deficiency, asthma, interstitial lung disease, and COVID-19 (Coronavirus Disease 2019).

36-41. (canceled)

42. The method according to claim 35, wherein the pulmonary disease is cystic fibrosis.

43. The method according to claim 42, wherein the heterologous nucleic acid comprises a nucleotide sequence selected from SEQ ID NO:30 or SEQ ID NO:31 or a sequence at least 80%, at least 90%, at least 95% or at least 99% identical thereto.

44-45. (canceled)

46. An infectious rAAV comprising (i) a variant capsid protein comprising a peptide insertion selected from the group consisting of HDITKNI (SEQ ID NO:12), NQDYTKT (SEQ ID NO: 13), DNTVTRS (SEQ ID NO:14), SNSVQSI (SEQ ID NO:15), NSTRHTD (SEQ ID NO: 16), TNRTSPD (SEQ ID NO:17), LAHDITKNIA (SEQ ID NO:40), LANQDYTKTA (SEQ ID NO: 41), LADNTVTRSA (SEQ ID NO:42), LASNSVQSIA (SEQ ID NO:43), LANSTRHTDA (SEQ ID NO:44), LATNRTSPDA (SEQ ID NO:45) and (ii) a heterologous nucleic acid comprising a nucleotide sequence encoding a gene selected from CFTR or a biologically active portion thereof and SERPINA1 or a biologically active portion thereof.

47-49. (canceled)

50. A pharmaceutical composition comprising the rAAV according to claim 46 and a pharmaceutically acceptable carrier.

51-74. (canceled)
